(19) United States
(12) Patent Application Publication
(10) Pub. No.: US 2025/0367222 A1
(43) Pub. Date: Dec. 4, 2025

(54) TREATMENTS FOR CANCERS DRIVEN BY DNAJB1-PRKACA GENE FUSIONS

(71) Applicant: Wisconsin Alumni Research Foundation, Madison, WI (US)

(72) Inventor: Sean Ronnekleiv-Kelly, Madison, WI (US)

(73) Assignee: Wisconsin Alumni Research Foundation, Madison, WI (US)

(21) Appl. No.: 19/178,455

(22) Filed: Apr. 14, 2025

Related U.S. Application Data
(60) Provisional application No. 63/634,239, filed on Apr. 15, 2024.

(51) Int. Cl. A61K 31/675; A61K 31/4162; A61K 31/4439; A61K 31/444; A61K 31/519; A61P 35/00

(52) U.S. Cl. CPC A61K 31/675; A61K 31/4162; A61K 31/4439; A61K 31/444; A61K 31/519; A61P 35/00

(57) ABSTRACT

Treatments for cancers driven by DNAJB1-PRKACA gene fusions, including fibrolamellar carcinoma (FLC) and other cancers, specifically, with the use of CDK7 inhibitors either alone or in combination with other agents, such as CDK9 inhibitors.

### CROSS-REFERENCE TO RELATED APPLICATIONS

Priority is hereby claimed to U.S. Provisional Application 63/634,239, filed Apr. 15, 2024, which is incorporated herein by reference in its entirety.

### STATEMENT REGARDING FEDERALLY SPONSORED RESEARCH

This invention was made with government support under W81XWH-22-1-0847 awarded by the Defense Health Agency/Medical Research and Development Branch (DHA/MRDB). The government has certain rights in the invention.

### SEQUENCE LISTING

The instant application contains a Sequence Listing which has been submitted in XML format and is hereby incorporated by reference in its entirety. The XML copy, created on Apr. 8, 2025, is named USPTO—09824595-P240225US02—SEQ_LIST.XML and is 10,668 bytes in size.

### FIELD OF THE INVENTION

The invention is directed to treatments for cancers driven by DNAJB1-PRKACA gene fusions, including fibrolamellar carcinoma (FLC) and other cancers, specifically, with the use of CDK7 inhibitors either alone or in combination with other agents, such as CDK9 inhibitors.

### BACKGROUND

A unique gene fusion created by a ˜400 kbp deletion between heat shock protein 40 (DNAJB1) and the catalytic subunit alpha of protein kinase A (PRKACA) (Honeyman et al. 2014), known as the DNAJB1-PRKACA gene fusion, is a proven driver mutation in a number of cancers, including the vast majority of human fibrolamellar carcinoma (FLC). The resultant chimeric protein (DNAJ-PKAc) is only present in tumor cells, indicating a somatic event (Kastenhuber et al. 2017, Oikawa et al. 2015, Dinh et al. 2020). Since its initial discovery, there has been inconclusive evidence of how DNAJ-PKAc drives cellular changes to promote neoplastic transformation and progression of FLC. Concordantly, there have been concerted efforts to understand how DNAJB1-PRKACA drives tumor development and mechanisms of treatment resistance so that new and effective therapies can be identified (Dinh et al. 2020, Neumayer et al. 2023). For instance, the chimeric protein demonstrates enhanced PKA activity compared to native PKA in response to cAMP (Honeyman et al. 2014), but simply inhibiting PKA is not a viable therapeutic option in FLC given the critical nature of PKA in normally functioning cells (i.e., no therapeutic window) (Bauer et al. 2022).

Treatments of DNAJB1-PRKACA-driven cancers are needed.

### SUMMARY OF THE INVENTION

One aspect of the invention is directed to methods of treating a cancer in a subject. The methods can comprise administering a CDK7 inhibitor to the subject in an amount effective to treat the cancer. The cancer is preferably a DNAJB1-PRKACA gene fusion-driven cancer.

In some versions, the cancer comprises a liver cancer, a pancreatic cancer, a cholangiocarcinoma (bile duct cancer), or a combination thereof.

In some versions, the cancer comprises fibrolamellar hepatocellular carcinoma.

In some versions, the CDK7 inhibitor comprises SY-5609, YKL-5-124, samuraciclib, or an enantiomer, a pharmaceutically acceptable salt, and/or a solvate of SY-5609, YKL-5-124, or samuraciclib.

In some versions, the methods further comprise administering to the subject one or more active agents other than the CDK7 inhibitor.

In some versions, the one or more additional active agents comprise one or more of a CDK9 inhibitor and a B-cell lymphoma-extra large (Bcl-xL) inhibitor.

In some versions, the one or more additional active agents comprise a CDK9 inhibitor. In some versions, the CDK9 inhibitor comprises VIP-152, NVP-2, or an enantiomer, a pharmaceutically acceptable salt, and/or a solvate of VIP-152 or NVP-2.

In some versions, the one or more additional active agents comprise a BCL-xL inhibitor. In some versions, the BCL-xL inhibitor comprises A1331852 or an enantiomer, a pharmaceutically acceptable salt, and/or a solvate thereof.

In some versions, the CDK7 inhibitor is administered to the subject within a week of administering at least one of the one or more additional active agents.

In some versions, the CDK7 inhibitor and at least one of the one or more additional active agents are simultaneously administered to the subject.

In some versions, the CDK7 inhibitor and at least one of the one or more additional active agents are administered in a single composition comprising both the CDK7 inhibitor and the least one of the one or more additional active agents.

Another aspect of the invention is directed to a composition comprising a CDK7 inhibitor and one or more additional active agents other than the CDK7 inhibitor.

In some versions, the CDK7 inhibitor comprises SY-5609, YKL-5-124, samuraciclib, or an enantiomer, a pharmaceutically acceptable salt, and/or a solvate of SY-5609, YKL-5-124, or samuraciclib.

In some versions, the one or more additional active agents comprise one or more of a CDK9 inhibitor and a B-cell lymphoma-extra large (Bcl-xL) inhibitor.

In some versions, the one or more additional active agents comprise a CDK9 inhibitor. In some versions, the CDK9 inhibitor comprises VIP-152, NVP-2, or an enantiomer, a pharmaceutically acceptable salt, and/or a solvate of VIP-152 or NVP-2.

In some versions, the one or more additional active agents comprise a BCL-xL inhibitor. In some versions, the BCL-xL inhibitor comprises A1331852 or an enantiomer, a pharmaceutically acceptable salt, and/or a solvate thereof.

The objects and advantages of the invention will appear more fully from the following detailed description of the preferred embodiment of the invention made in conjunction with the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1D. Gene expression and protein analysis of human FLC samples. FIG. 1A. mRNA expression of various known super enhancer driven genes in FLC were assessed in human tumor samples (n=35) and paired normal liver samples (n=10) (**q<0.0001). FIG. 1B. Individual patient tumor samples with matched normal liver (FCF 82, 83 and 106) were evaluated for phosphorylated RPB-1 (Ser-2, -5, -7) and CDK7. Fibrolamellar cancer was confirmed by demonstrating presence (tumor) and absence (normal) of the DNAJ-PKAc oncoprotein. FIG. 1C. A phosphorylation index was calculated for each sample and phosphorylation level is presented as Phosphorylation index (Tumor)/Phosphorylation index (Adjacent) for RPB-1 (Ser-2, -5, -7) and CDK7. 7-9 sets of samples are shown from FLC tumor and adjacent normal tissue samples. FIG. 1D. CDK7 forms a trimeric complex with cyclin H and MAT1 to phosphorylate serine-5 (preferentially) and serine-7 residues of a 52 heptad repeat in RNA Polymerase II. Similarly, CDK9 dimerizes with Cyclin T to preferentially phosphorylate serine-2 residues. SY-5609 and YKL-5-124 inhibit CDK7 activity, similarly VIP-152 and NVP-2 inhibit CDK9 activity. [Image created with Biorender.com].

FIGS. 2A-2F. Generation of a model to understand pathogenic mechanisms of DNAJB1-PRKACA in FLC. FIG. 2A. Parent hepatoblastoma cells (HepG2) were transfected with a dual-guide RNA-CAS9 plasmid containing an eGFP tag, to allow for cell sorting of HepG2 cells that took up the plasmid. Each cell was deposited in a single well of a 96-well plate and clonally expanded. FIG. 2B. The guide RNAs (gRNA1: CAGGAGCCGACCCCGTTCGT (SEQ ID NO:1), gRNA2: GTAGACGCGGTTGCGCTAAG (SEQ ID NO:2)) directed CAS9 to induce a double-strand break at intron 1 of DNAJB1 and intron 1 of PRKACA, resulting in 400 kb deletion and chromosomal rearrangement to generate the DNAJB1-PRKACA oncogene fusion. FIG. 2C. After expansion, genomic DNA from each clone was assessed via PCR for presence of DNAJB1-PRKACA gene fusion, and six clones manifested appropriately sized PCR products (DNA bands) pertaining to the forward and reverse primers across the breakpoint. FIG. 2D. The PCR product from each clone and DNAJB1-PRKACA mRNA transcript (across the breakpoint) was sequenced to determine the precise sequence by CRISPR gene editing. FIG. 2E. Assessment of expression levels of native genes DNAJB1 and PRKACA (light bars), as well as FLC-specific genes including DNAJB1-PRKACA fusion, SLC16A14 and LINC00473 (dark bars) was performed. Shown is relative fold-change (error bars represent standard deviation) compared to parent HepG2 cells, with four biological replicates per clone. FIG. 2F. Clone samples and parent HepG2 cells were also evaluated for generation of fusion oncoprotein (DNAJ-PKAc) and native PKA (B-Actin control). Clone selection for subsequent assays was based on these rigorous validation metrics (FIGS. 2A-2F) yielding H33 (* arrow in FIG. 2E) and H12 (#arrow in FIG. 2E) as the selected clones.

FIGS. 3A-3E. CDK7 regulation of RNA Polymerase II phosphorylation and super enhancer gene expression. FIG. 3A. Protein analysis for phosphorylated RNA polymerase II (serine-2, serine-5, serine-7), CDK2 and CDK7 was performed and phosphorylation levels were quantified using ImageJ. Shown are two separate biological replicates for each line. FIG. 3B. DNAJB1-PRKACA expressing H33 cells were treated with a selective CDK7 inhibitor, SY5609 (100 nM, 1 μM, 10 μM), or DMSO control (0) for 24 hours. Known substrate targets of CDK7 were assessed including RNA Pol II CTD (ser-2, -5, and -7), Thr160 phosphorylated CDK2(pCDK2) and Thr170 phosphorylated CDK7 (pCDK7). FIG. 3C. RNA sequencing of HepG2 cells and H33 cells (six biological replicates per group) were evaluated for prominent super enhancer associated genes, including SLC16A14 and LINC00473. Findings from the H33 clone were confirmed in a separate DNAJB1-PRKACA expressing clone (H12). FIGS. 3D-3E. To assess for CDK7-dependent expression of FLC-specific genes, H33 cells were treated with SY-5609 (1 μM-300 μM) and levels of mRNA expression (RT-qPCR) versus DMSO control were evaluated, including SLC16A14 and LINC00473. This was repeated with a separate covalent-binding selective and specific CDK7 inhibitor (YKL-5-124). Shown are three biological replicates per drug dose per mRNA (**p<0.0001, *p<0.05). [Image created with Biorender.com].

FIGS. 4A-4E. CDK7 is a novel therapeutic target in DNAJB1-PRKACA expressing cells. FIG. 4A. To assess for CDK7 effect on cell viability, HepG2 cells and H33 cells underwent 48 hr drug treatment with either SY5609 (1 pM-5 μM) or YKL-5-124 (100 pM-10 μM). Percent viability was determined by normalizing to control (DMSO treated). To confirm the findings in the DNAJB1-PRKACA expressing H33 cells, a separate clone (H12) was tested with the same drugs over the same dose range. In each figure, the LC₅₀ (IC₅₀) is represented by the straight line. FIG. 4B. HepG2 cells and H33 cells were synchronized and treated with either DMSO or SY5609 (1 μm) for 24 hours. Percent of cells in G0/G1, S and G2/M were determined by flow cytometry. Shown are four biological replicates per cell line per treatment (*p=0.0015, **p<0.0001). FIG. 4C. HepG2 cells and H33 cells were treated with DMSO (control) or increasing doses of SY5609 for 24 hours and Caspase 3/7 activity measured. To confirm the increased apoptotic activity in the H33 cells, a separate clone (H12) was utilized. (*p<0.0001, for H33 vs HepG2 and H12 vs HepG2). To validate the results, PARP and cleaved-PARP (marker for apoptosis) protein were evaluated in HepG2 and H33 cells, using two separate antibodies that either recognize both PARP and cleaved-PARP (top bands) or only cleaved-PARP alone (bottom band). FIG. 4D. Primary human hepatocytes (PHHs) isolated fresh from human donor liver transplant specimens and H33 cells were treated with DMSO, SY5609 100 nM, or SY5609 1 μM for 24- and 48-hours. The percent of viable cells was determined by normalizing to DMSO control. There were four biological replicates per group per time/treatment dose (*p=0.02, **p =0.01, ***p <0.0001). FIG. 4E. PHHs and H33 cells were treated with SY5609 (100 pM-5 μM) for 48-, 72- or 120-hours and percent viability assessed, normalized to DMSO control. The LC₅₀ (IC₅₀) is demarcated by the solid line. There were four biological replicates for each drug dose at each time point for each line (PHH and H33).

FIGS. 5A-5D. CDK7 inhibition is lethal to human FLC. FIG. 5A. An FLC cell line derived from human FLC (FLC-H) was grown for six days (control) or treated with DMSO (Control DMSO) versus SY5609 at two separate doses (500 nM and 1 μM). Survived cells were quantified at day zero and day six. Additionally, the day six percent survival compared to controls was calculated. Shown are two separate experiments with three biological replicates per experiment (*p<0.01, **p<0.001 versus Control and Control DMSO). FIG. 5B. In a separate experiment, an FLC cell line was derived from a patient's FLC liver tumor (FLC1025), and another discrete cell line was derived from another patient's metastatic FLC tumor implants (FLCMet). Each line was treated with SY5609 (10 nM-10 μM). Percent survival was determined normalized to DMSO control, with three biological replicates per dose. Shown is dose response with curve (and 95% CI) and LC₅₀ (IC₅₀) demonstrated by the straight line (LC₅₀ FLC1025˜300 nM, LC₅₀ FLCMet˜20 nM). FIG. 5C. Human tissue slices derived from a patient with FLC (FLC217) were treated with DMSO or SY5609 (500 nM), and percent viability determined compared to DMSO control (**p=0.003). FIG. 5D. In a separate experiment, tissue slices derived from human FLC grown in a patient derived xenograft (PDX) model were treated with DMSO, SY5609 (100 nM, 500 nM and 1 μM) and Staurosporine (STS) 500 nM (positive control). There were 3-4 biological replicates per group (*p<0.01, **p<0.001).

FIGS. 6A-6D. Synergistic combination between CDK7 and CDK9 inhibition in vitro. FIG. 6A. DNAJB1-PRKACA expressing H33 cells were treated with SY-5609 alone, VIP-152 alone, or in combination for 24 hours and protein for phosphorylated RPB-1 (Ser-2, -5, -7) was measured and quantified. FIG. 6B. Expression of SLC16A14 and LINC00473 were determined in H33 cells treated with SY-5609 alone, VIP-152 alone or in combination. FIGS. 6C-6D. Synergistic response to combination therapy of SY-5609 and VIP-152 in H33 cells was determined with potent reduction in percent survival (normalized to DMSO control). Depicted is a dose response curve which demonstrates shift of the curve to the left for SY-5609 with increasing concentration of VIP-152 up to a dose of 30 nM VIP-152. The drug combination showed strong synergy using all metrics including HSA (mean 18.73, p=6.31e-5), Bliss (mean 14.55, p=3.46e-4), Loewe (mean 15.48, p=1.98e-4), and ZIP (mean 13.54, p =6.04e-4). The strongest synergistic doses occurred at 30 nM SY-5609+30nM VIP152 (synergy score ˜38) and 10nM SY-5609+30 nM VIP152 (synergy score ˜37). [Image created with Biorender.com].

FIGS. 7A-7B. CDK7 and CDK9 inhibition in an organoid model. FIG. 7A. Tissue from a patient with FLC (FLC4-PDX) was propagated and developed into a patient derived cancer organoid model, FLC4 (organoid). Western blot shows the presence of DNAJ-PKAc oncoprotein and phosphorylated RPB-1 (Ser-2, -5, -7) in the organoid compared to immortalized human hepatocyte (IHH, n=4) which displays only native PKAc and low levels of phosphorylated RPB-1. Phosphorylated RPB-1 (Ser-2, -5, -7) was measured and quantified. FIG. 7B. RNA was harvested from organoids treated with SY-5609 (1 nM, 3 nM, 10 nM, 30 nM) and VIP-152 (30 nM, 100 nM, 300 nM and 500 nM) and SLC16A14 and LINC00473 expression were determined by qPCR relative to DMSO control (n=3). [Image created with Biorender.com].

FIGS. 8A-8C. Synergistic combination of CDK7 and CDK9 inhibition in an organoid model. FIG. 8A. FLC organoids were treated with SY-5609 alone, VIP-152 alone or in combination and a western blot was performed showing phosphorylated RPB-1 (Ser-2, -5, -7). Protein levels were measured and quantified. FIG. 8B. Organoids were treated with SY-509 alone and combination with VIP-152 at various doses showing a dose dependent decrease in cell survival as compared to DMSO. C. RNA was harvested from organoids treated with combination SY-5609and VIP-152 (1 nM/10 nM, 10 nM/10 nM and 100 nM/100 nM) therapy for combination CDK7 and CDK9 inhibition and expression of SLC16A14 and LINC00473 were determined (n=3). [Image created with Biorender.com].

FIG. 9. Protein analysis of human FLC samples. Individual patient tumor samples with matched normal liver (FCF 106, 110, 114, 87 and 89) were evaluated for phosphorylated RPB-1 (Ser-2, -5, -7) and CDK7. Fibrolamellar cancer was confirmed by demonstrating presence (tumor) and absence (normal) of the DNAJ-PKAc oncoprotein in samples FCF 106, 114 and 109. The absence of the DNAJ-PKAc oncoprotein in histologically confirmed samples FCF 110 and 87 suggests these patients may have had ATP1B1-PRKACA or ATP1B1-PRKACB mutations, which have also been shown to cause FLC. Samples FCF 84 and 57 were evaluated for phosphorylated RPB-1 (Ser-2, -5, -7), however, due to limitations of clinically resected tissue quality and quantity, complete protein analysis for CDK7 and pCDK7 in these samples was unable to be completed. Phosphorylation indices for these samples are represented in FIG. 1C. Phosphorylation indices for these samples are represented in FIG. 1C. [Image created with Biorender.com].

FIG. 10. KEGG pathway analysis. The top 500 differentially expressed genes in the human FLC (n=35) versus human normal liver (n=10) [top panel], and the top 500 differentially expressed genes between H33 cells (n=6) versus HepG2 cells (n=6) [bottom panel] were included in the KEGG pathway analysis. Of the thirteen enriched pathways in the human samples, six overlapped with the cell line enriched pathways including i) primary bile acid biosynthesis, ii) complement and coagulation cascades, iii) PPAR signaling pathway, iv) bile secretion, v) ECM-receptor interaction and vi) metabolic pathways. Fold enrichment is shown on the x-axis with specific pathway on the left y-axis. The number of genes involved in each pathway is depicted by circle size and the minus log-base-10 false discovery rate (FDR) is depicted by color.

FIG. 11. FLC-specific gene expression in response to SY-5609 treatment. VCAN and OAT are two additional genes that have been consistently identified as overexpressed in human FLC samples, however their expression was not impacted by SY-5609 treatment.

FIGS. 12A-12B. siRNA dose response in HepG2 and H33 cell lines. FIG. 12A. HepG2 cells and H33 cells were treated with siControl (300 pM-100 nM), siCDK7-1 (300 pM-100 nM) or siCDK7-2 (10 pM-100 nM). Percent viability was determined by normalizing to control (DMSO) treated cells. siCDK7-1 is Silencer select siRNA (s2830) targeting exon 3 of CDK7 and siCDK7-2 is Stealth RNAi (VHS40192) targeting exon 7 of CDK7. The LC₅₀ (IC₅₀) value is demarcated by the solid black line in the siCDK7 treated cells (For siCDK7-1 the IC₅₀˜3 nM H33 cells vs ˜50 nM HepG2 cells, for siCDK7-2 the IC₅₀˜500 pM H33 cells vs ˜10 nM HepG2 cells). FIG. 12B. Immunoblot was performed for CDK7 in H33 cells after 48 hour treatment with siControl, siCDK7-1 and siCDK7-2. Relative CDK7 expression is displayed numerically and quantified as CDK7 protein level/β-actin level. [Image created with Biorender.com].

FIGS. 13A-13B. Triptolide dose response in HepG2 and H33 cell lines. FIG. 13A. HepG2 and H33 cells were treated with Triptolide (100 pM-100 nM), an inhibitor of TFIIH, and percent viability was determined by normalizing to control (DMSO) treated cells. There was no significant difference in percent viability between HepG2 and H33. FIG. 13B. The expression of FLC specific genes is shown in H33 cells in response to treatment with Triptolide. There is a statistically significant decrease in expression at high doses. [Image created with Biorender.com].

FIGS. 14A-14B. Synergistic combination therapy between VIP-152 and SY-5609 in primary human hepatocytes. Synergistic response to combination therapy of SY-5609 and VIP-152 in H33 cells was compared to primary human hepatocytes (PHH). There was a potent reduction in percent survival in H33 without significant cell death in PHH (normalized to DMSO control). The drug combination showed some synergy in PHH including HSA score of 4.138.

FIGS. 15A-15C. Synergistic combination therapy between NVP-2 and SY-5609 in vitro. FIG. 15A. DNAJB1-PRKACA expressing H33 cells were treated with SY-5609 alone, NVP-2 alone or in combination for 24 hours and protein for phosphorylated RPB-1 (Ser-2, -5, -7) was measured and quantified. FIG. 15B. Expression of SLC16A14 and LINC00473 were determined in H33 cells treated with SY-5609 alone, NVP-2 alone or in combination. FIG. 15C. Synergistic response to combination therapy of SY-5609 and NVP-2 in H33 cells was determined and demonstrated significant synergy with a score of 10.355 (HSA metric).

FIG. 16. Expression of FLC specific genes in human FLC organoids. Expression of SLC16A14, LINC00473 and VCAN was determined in human FLC organoids as compared to immortalized human hepatocytes (IHH).

FIGS. 17A-17F. CDK7 Inhibition is tumoricidal in a heterotopic patient cancer-derived xenograft (PDX) model of Fibrolamellar carcinoma (FLC). Ten immunodeficient mice (R2G2) were injected in the left flank with 1×10⁵ FLC PDX cells at 8-10 weeks of age. These cells were derived from a patient with FLC and harbor the DNAJB1-PRKACA gene fusion. Approximately 70 days following injection grossly palpable tumors were noted for each mouse and baseline measurements were taken to calculate starting volume for each tumor. Although all mice had the same number of tumor cells injected for tumor initiation, tumor volume varied between the mice and was calculated as follows:

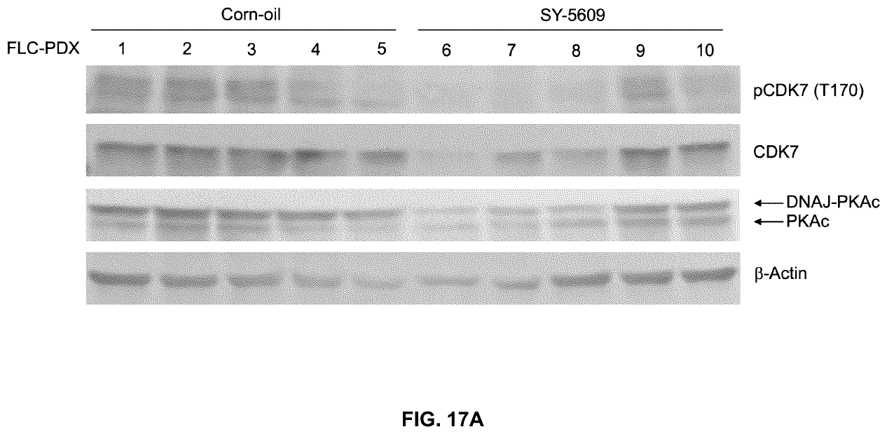

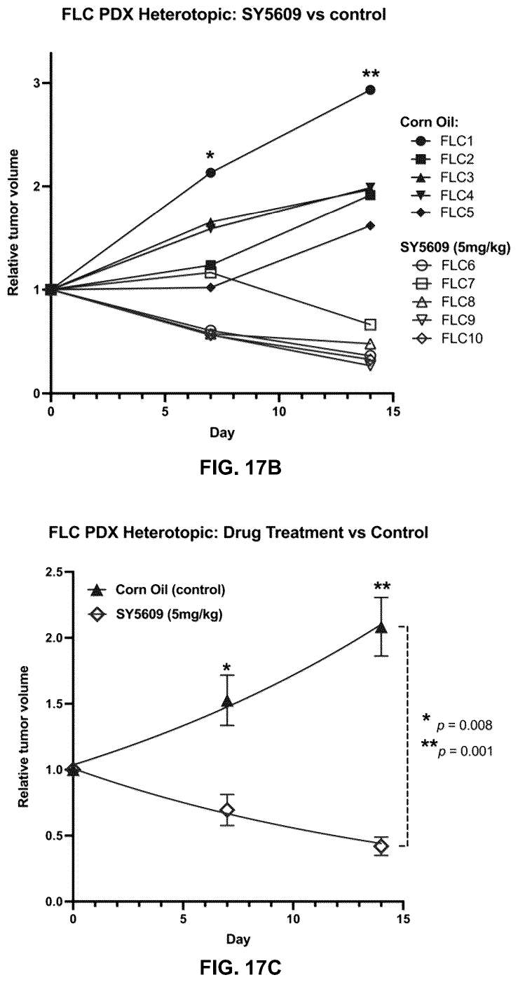

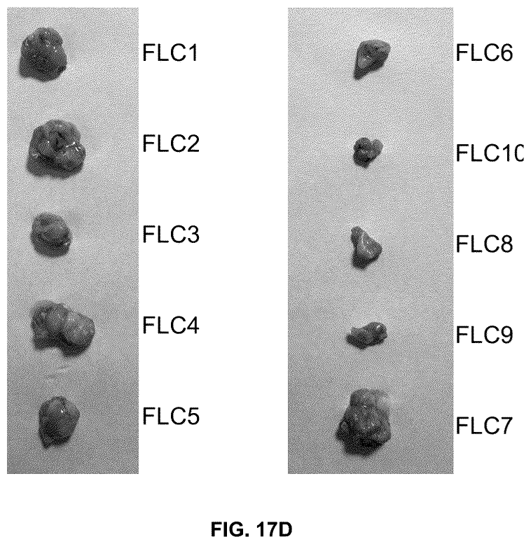

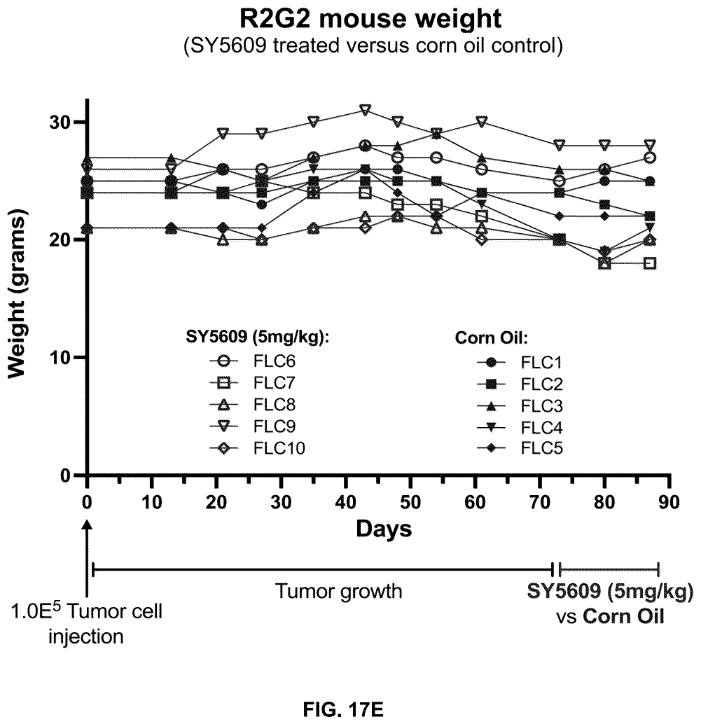

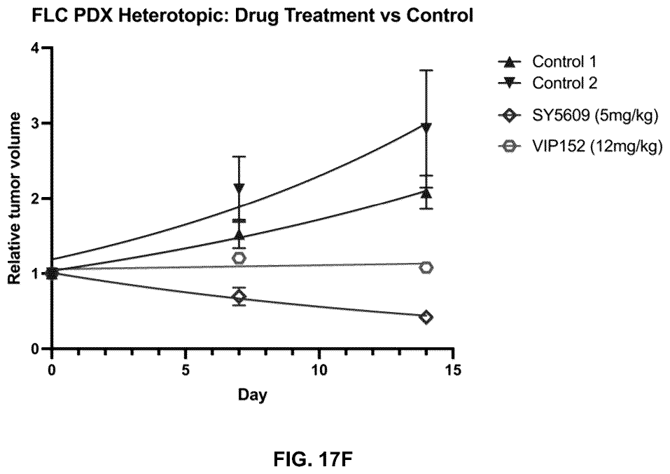

$$V = \frac{1}{2} * L * {W}^{2} ,$$

where L is the length of the tumor and W is the width of the tumor. Mice labeled FLC1-5 were administered corn oil vehicle control via oral gavage on days 1-14. Mice labeled FLC6-10 were administered the CDK7 inhibitor SY-5609 at a dose of 5 mg/kg in corn oil via oral gavage on days 1-14. Mice were sacrificed on day 15 after initiation of therapy, tumors removed, final tumor volumes calculated, and tumor preserved for molecular and histologic analysis. FIG. 17A. Western blot showing the expression of indicated proteins. FIG. 17B. Tumor volume is shown for each individual animal over the course of the treatment. Relative tumor volume is depicted in the y-axis because each tumor was a different volume at initiation of therapy and tumor trajectory is normalized to this starting value. Animals treated with corn oil are shown in solid shapes and animals treated with SY-5609 are shown in non-filled shapes. *p=0.008, **p=0.001 for corn oil versus SY-5609. FIG. 17C. Aggregate tumor volume data from the corn oil and SY-5609 treated mice is shown. Overall, there was a significant decrease in tumor volume at days 7 and 14 in animals treated with SY-5609 while animals treated with vehicle control showed increase in tumor volume. *p=0.008, **p=0.001 for corn oil versus SY-5609. FIG. 17D. Following sacrifice on day 15 tumors were removed and gross images of the tumors are displayed from control (FLC 1-5) and treatment (FLC 6-10) groups. FIG. 17E. Mouse weight data is depicted throughout the course of the study. Tumor initiation with 1×10⁵ tumor cells was followed by tumor growth for 73 days at which point mice were treated with SY5609 (blue) or corn oil (red). There were no mouse deaths or significant changes in weight; the mice appeared healthy throughout the course of the study. FIG. 17F. Experiment as shown in FIG. 17C but with VIP-152 treatment as well. Control: corn oil.

### DETAILED DESCRIPTION OF THE INVENTION

One aspect of the invention is directed to methods of treating a cancer in a subject.

The cancer is preferably a DNAJB1-PRKACA gene fusion-driven cancer. “DNAJB1-PRKACA gene fusion-driven cancer” refers to a cancer to which a DNAJB1-PRKACA gene fusion contributes to its etiology.

As used herein, “DNAJB1-PRKACA gene fusion” refers to a fusion of the genes for heat shock protein 40 (DNAJB1) and the catalytic subunit alpha of protein kinase A (PRKACA), in any configuration in the genome of one or more cells in a subject. The fusion can result from genomic deletion of bases between the genes and, typically, portions of the genes themselves. In some versions, the DNAJB1-PRKACA gene fusion gene fusion is created by a ˜400 kbp deletion between DNAJB1 and PRKACA (Honeyman et al. 2014).

DNAJB1-PRKACA gene fusion-driven cancers are well known in the art. These include various liver cancers, pancreatic cancers, and cholangiocarcinomas (bile duct cancers), among others. Exemplary DNAJB1-PRKACA gene fusion-driven cancers include comprises fibrolamellar hepatocellular carcinoma, pancreatobiliary neoplasms, intraductal oncocytic papillary neoplasms, intraductal papillary mucinous neoplasms, pancreatic ductal adenocarcinoma, and intrahepatic cholangiocarcinoma, among others. See, e.g., Singhi et al. 2020 (Singhi A D, Wood L D, Parks E, Torbenson M S, Felsenstein M, Hruban R H, Nikiforova M N, Wald A I, Kaya C, Nikiforov Y E, Favazza L, He J, McGrath K, Fasanella K E, Brand R E, Lennon A M, Furlan A, Dasyam A K, Zureikat A H, Zeh H J, Lee K, Bartlett D L, Slivka A. Recurrent Rearrangements in PRKACA and PRKACB in Intraductal Oncocytic Papillary Neoplasms of the Pancreas and Bile Duct. Gastroenterology. 2020 February; 158 (3):573-582.e2) and Vyas et al. 2020 (Vyas M, Hechtman J F, Zhang Y, Benayed R, Yavas A, Askan G, Shia J, Klimstra D S, Basturk O. DNAJB1-PRKACA fusions occur in oncocytic pancreatic and biliary neoplasms and are not specific for fibrolamellar hepatocellular carcinoma. Mod Pathol. 2020 April; 33 (4):648-656).

The methods of the invention can comprise administering a cyclin-dependent kinase 7 (CDK7) inhibitor to the subject. CDK7 is a member of the cyclin-dependent protein kinase (CDK) family. The protein forms a trimeric complex with cyclin H and MAT1, which functions as a Cdk-activating kinase (CAK). It is an essential component of the transcription factor TFIIH, that is involved in transcription initiation and DNA repair. CDK7 is thought to serve as a direct link between the regulation of transcription and the cell cycle. In humans, CDK7 is encoded by the CDK7 gene.

A large number of CDK7 inhibitors are known in the art. Examples include SY-5609 (CAS No. 2417302-07-7), YKL-5-124 (CAS No. 1957203-01-8), samuraciclib (CAS Nos. 1805789-54-1 (HCl), 1805833-75-3 (Free Base)), Q901, XL102, roscovitine, BS-181, LDC3140, LDC4297, THZ1, THZ2, SY-1365, SY-5609, PF-3758309, YKL-1-116, B2, A86, 9q, SZ-015093, APPAMP-003, APPAMP-004, H-APPAMP-015, PPA-024, I-55, LY3405105, SZ-015249, SZ-015268, VII-3, among many others. See, e.g., Kovalová et al. 2023 (Kovalová M, Baraka J P, Mik V, Jorda R, Luo L, Shao H, Kryštof V. A patent review of cyclin-dependent kinase 7 (CDK7) inhibitors (2018-2022). Expert Opin Ther Pat. 2023 February; 33 (2):67-87), U.S. Pat. No. 10,738,067, US 2021/0401859 A1, and US 2021/0403495 A1, among others. Additional examples include enantiomers, pharmaceutically acceptable salts, and/or solvates of any of the CDK7 inhibitors provided herein. An exemplary structure for SY-5609 is:

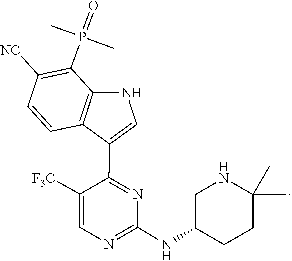

An exemplary structure for YKL-5-124 is:

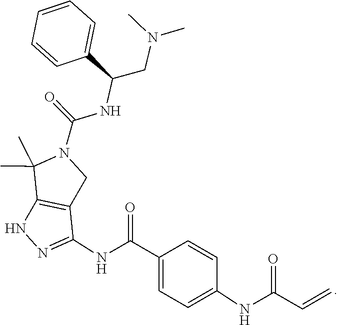 [SMILES: C=CC(=O)Nc1ccc(C(=O)Nc2n[nH]c3c2CN(C(=O)N[C@H](CN(C)C)c2ccccc2)C3(C)C)cc1]

An exemplary structure for samuraciclib is:

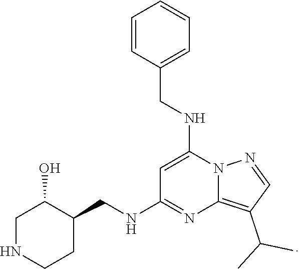 [SMILES: CC(C)c1cnn2c(NCc3ccccc3)cc(NC[C@H]3CCNC[C@@H]3O)nc12]

Administering a CDK7 inhibitor alone can be effective to treat cancers, such as DNAJB1-PRKACA gene fusion-driven cancers. Accordingly, the CDK7 inhibitor can be administered to a subject in an amount effective to treat the cancer.

Some embodiments further comprise administering to the subject one or more additional active agents other than the CDK7 inhibitor. As shown in the following examples combining various additional active agents with the CDK7 inhibitor can produce synergistic effects in the treatment of cancers, such as DNAJB1-PRKACA gene fusion-driven cancers. Accordingly, in some versions, the one or more additional active agents are administered in a synergistic amount. “Synergistic amount” as used herein refers to an amount effective to produce synergistic effect in the treatment of the cancer.

In some versions, the one or more additional active agents include a cyclin-dependent kinase 9 (CDK9) inhibitor. CDK9 is a cyclin-dependent kinase associated with P-TEFb. CDK9 is a member of the cyclin-dependent kinase (CDK) family. CDK9 was found to be a component of the multiprotein complex TAK/P-TEFb, which is an elongation factor for RNA polymerase II-directed transcription and functions by phosphorylating the C-terminal domain of the largest subunit of RNA polymerase II. This protein forms a complex with and is regulated by its regulatory subunit cyclin T or cyclin K. In humans, CDK9 is encoded by the CDK9 gene.

A large number of CDK9 inhibitors are known in the art. Examples include VIP-152 (CAS No. 1610408-97-3 (S-isomer)), NVP-2 (CAS No. 1263373-43-8), alvocidib/DSP-2033/flavopiridol, dinaciclib, SNS-032, RGB286638, zotiraciclib (TG02), atuveciclib (BAY-1143572), BAY-1251152, AZD4573, AZD5576, AT7519, CYC065, nanoflavopiridol, seliciclib (CYC202), TG02, TP-1287, VS2-370, and voruciclib (formerly P1446A-05), among others. See, e.g., Alsfouk 2021 (Alsfouk A. Small molecule inhibitors of cyclin-dependent kinase 9 for cancer therapy. J Enzyme Inhib Med Chem. 2021 December; 36 (1):693-706), U.S. Pat. No. 10,738,067, US 2021/0401859 A1, and US 2021/0403495 A1, WO 2023/057813 A1, WO 2023/009481 A1, WO 2019/154177, WO 2014/060376, WO 2015/150273, WO 2013/037896, WO 2018/177899, WO 2017/055196, WO 2015/136028, WO 2014/076091, WO 2016/059011, WO 2016/059086, and WO 2015/001021, U.S. Pat. No. 7,943,629, Byrne et al. 2018 (Byrne M, Frattini M G, Ottmann O G, et al. Phase I study of the PTEFb inhibitor BAY 1251152 in patients with acute myelogenous leukemia. Blood 2018; 132:4055-4055), among others. Additional examples include enantiomers, pharmaceutically acceptable salts, and/or solvates of any of the CDK9 inhibitors provided herein.

An exemplary structure for VIP-152 is:

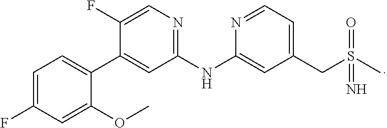 [SMILES: COc1cc(F)ccc1-c1cc(Nc2cc(CS(C)(=N)=O)ccn2)ncc1F]

An exemplary structure for NVP-2 is:

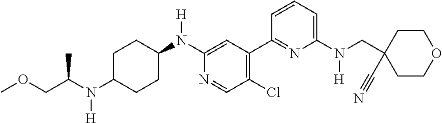

In some versions, the one or more additional active agents include a B-cell lymphoma-extra large (Bcl-xL) inhibitor. Bcl-xL, encoded by the BCL2-like 1 gene, is a transmembrane molecule in the mitochondria. It is a member of the Bcl-2 family of proteins, and acts as an anti-apoptotic protein by preventing the release of mitochondrial contents such as cytochrome c, which leads to caspase activation and ultimately, programmed cell death. Exemplary Bcl-xL inhibitors include A1331852 (A-1331852) (CAS No. 1430844-80-6), APG-1252, BH3I-1, DT2216, 10-deacetyl-7-xylosyl paclitaxel, flavokawain A, A-1155463 dihydrochloride, navitoclax (ABT-263), WEHI-539, APG-2575 (lisaftoclax), ABT-737, sabutoclax, gossypol, (R)-(−)-gossypol acetic acid, TW-37, gambogic acid, berberine chloride (NSC 646666), and berberine chloride hydrate, among others. Additional examples include enantiomers, pharmaceutically acceptable salts, and/or solvates of any of the Bcl-xL inhibitors provided herein. An exemplary structure for A1331852 is:

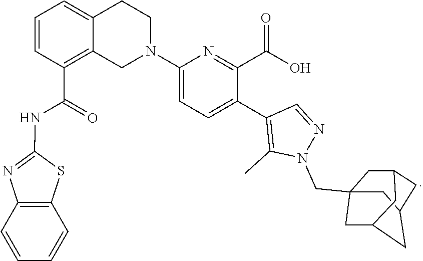 [SMILES: Cc1c(-c2ccc(N3CCc4cccc(C(=O)Nc5nc6ccccc6s5)c4C3)nc2C(=O)O)cnn1CC12CC3CC(CC(C3)C1)C2]

Further additional active agents that can be used in combination with the CDK7 inhibitor include Bcl-2 inhibitors such as APG-1252, APG-2575, BP1002 (prexigebersen), the antisense oligonucleotide known as oblimersen (G3139), S55746/BCL201, and venetoclax, among others; hormone receptor (e.g., estrogen receptor) degradation agents, such as fulvestrant; FLT3 (FMS-like tyrosine kinase 3) inhibitors such as CDX-301, CG-806, CT053PTSA, crenolanib (e.g., crenolanib besylate), ENMD-2076, FF-10101-01, FLYSYN, gilteritinib (ASP2215), HM43239, lestautinib, ponatinib, NMS-088, sorafenib, sunitinib, pacritinib, pexidartinib/PLX3397, quizartinib, midostaurin, SEL24, SKI-G-801, and SKLB1028, among others; PARP inhibitors such as olaparib, rucaparib, talazoparib, veliparib (ABT-888), and niraparib, among others; BET inhibitors such as ABBV-075, BAY-299, BAY-1238097, BMS-986158, CPI-0610, CPI-203, FT-1101, GS-5829, GSK-2820151, GSK-525762, I-BET151, I-BET762, INCB054329, JQ1, MS436, OTX015, PFI-1, PLX51107, RVX2135, TEN-010, and ZEN-3694, among others; platinum-based therapeutic agents such as cisplatin, oxaliplatin, nedaplatin, carboplatin, phenanthriplatin, picoplatin, satraplatin (JM216), and ortriplatin tetranitrate, among others; CDK4/6 inhibitors such as BPL1178, G1T38, palbociclib, ribociclib, ON 123300, trilaciclib, and abemaciclib, among others; MEK inhibitors such as trametinib, cobimetinib, and binimetinib, among others; and phosphoinositide 3-kinase (PI3 kinase) inhibitors, optionally of Class I (e.g., Class IA) and/or optionally directed against a specific PI3K isoform, such as idelalisib, copanlisib, duvelisib, and alpelisib, among others.

In some versions, the CDK7 inhibitor is administered to the subject within 4 weeks, 3 weeks, 2 weeks, 1 week, 6 days, 5 days, 4 days, 3 days, 2 days, or 1 day of administering at least one of the one or more additional active agents. In some versions, the CDK7 inhibitor is administered to the subject within 4 weeks, 3 weeks, 2 weeks, 1 week, 6 days, 5 days, 4 days, 3 days, 2 days, or 1 day of administering each of the one or more additional active agents. In some versions, the CDK7 inhibitor and at least one of the one or more additional active agents are simultaneously administered to the subject. In some versions, the CDK7 inhibitor and each of the one or more additional active agents are simultaneously administered to the subject. In some versions, the CDK7 inhibitor and at least one of the one or more additional active agents are administered in a single composition comprising both the CDK7 inhibitor and the least one of the one or more additional active agents. In some versions, the CDK7 inhibitor and each of the one or more additional active agents are administered in a single composition comprising both the CDK7 inhibitor and each of the one or more additional active agents.

In some versions, the subject is suspected of having or confirmed to have a DNAJB1-PRKACA gene fusion. In some versions, the subject is confirmed to have a DNAJB1-PRKACA gene fusion. Some methods of the invention accordingly comprise detecting a DNAJB1-PRKACA gene fusion in the subject. The detection can occur prior to or after administering the CDK7 inhibitor and, optionally, the one or more addition active agents. Methods of detecting the DNAJB1-RKACA gene fusion are well known in the art.

Other aspects of the invention are directed to compositions, such as pharmaceutical compositions, comprising a CDK7 inhibitor and one or more of the additional active agents other than the CDK7 inhibitor. The composition can comprise the CDK7 inhibitor and any one or more additional active agents, such as any one or more additional active agents disclosed herein. The composition can also include a pharmaceutically acceptable carrier.

The following definitions apply to the compositions, methods, and uses described herein unless the context clearly indicates otherwise, and it is to be understood that the claims may be amended to include language within a definition as needed or desired. Moreover, the definitions apply to linguistic and grammatical variants of the defined terms (e.g., the singular and plural forms of a term), and some linguistic variants are particularly mentioned below (e.g., “administration” and “administering”).

The term “about,” when used in reference to a value, signifies any value or range of values that is plus-or-minus 10% of the stated value (e.g., within plus-or-minus 1%, 2%, 3%, 4%, 5%, 6%, 7%, 8%, 9% or 10% of the stated value). For example, a dose of about 10 mg means any dose as low as 10% less than 10 mg (9 mg), any dose as high as 10% more than 10 mg (11 mg), and any dose or dosage range therebetween (e.g., 9-11 mg; 9.1-10.9 mg; 9.2-10.8 mg; and so on). As another example, a prevalence rank in a population of about 80% means a prevalence rank of 72-88% (e.g., 79.2-80.8%). In case of doubt, “about X” can be “X” (e.g., about 80% can be 80%). Where a stated value cannot be exceeded (e.g., 100%), “about” signifies any value or range of values that is up to and including 10% less than the stated value (e.g., a purity of about 100% means 90%-100% pure (e.g., 95%-100% pure, 96%-100% pure, 97%-100% pure, etc.)). In the event an instrument or technique measuring a value has a margin of error greater than 10%, a given value will be about the same as a stated value when they are both within the margin of error for that instrument or technique.

The term “administration” and variants thereof, such as “administering,” refer to the administration of an active agent described herein, or a composition containing the active agent to a subject (e.g., a human patient) or system (e.g., a cell- or tissue-based system that is maintained ex vivo); as a result of the administration, the active agent or composition containing the active agent (e.g., a pharmaceutical composition) is introduced to the subject or system. In addition to compositions of the invention and additional active agents useful in combination therapies, items used as positive controls, negative controls, and placebos, any of which can also be a compound, can also be “administered.” One of ordinary skill in the art will be aware of a variety of routes that can, in appropriate circumstances, be utilized for administration to a subject or system. For example, the route of administration can be oral (i.e., by swallowing a pharmaceutical composition) or may be parenteral. More specifically, the route of administration can be bronchial (e.g., by bronchial instillation), by mouth (i.e., oral), dermal (which may be or comprise topical application to the dermis or intradermal, interdermal, or transdermal administration), intragastric or enteral (i.e., directly to the stomach or intestine, respectively), intramedullary, intramuscular, intranasal, intraperitoneal, intrathecal, intratumoral, intravenous (or intra-arterial), intraventricular, by application to or injection into a specific organ (e.g., intrahepatic), mucosal (e.g., buccal, rectal, sublingual, or vaginal), subcutaneous, tracheal (e.g., by intratracheal instillation), or ocular (e.g., topical, subconjunctival, or intravitreal). Administration can involve intermittent dosing (i.e., doses separated by various times) and/or periodic dosing (i.e., doses separated by a common period of time (e.g., every so many hours, daily (e.g., once daily oral dosing), weekly, twice per week, etc.)). In other embodiments, administration may involve continuous dosing (e.g., perfusion) for a selected time (e.g., about 1-2 hours).

The term “cancer” refers to a disease in which biological cells exhibit an aberrant growth phenotype characterized by loss of control of cell proliferation to an extent that will be detrimental to a patient having the disease. A cancer can be classified by the type of tissue in which it originated (histological type) and/or by the primary site in the body in which the cancer first developed. Based on histological type, cancers are generally grouped into six major categories: carcinomas; sarcomas; myelomas; leukemias; lymphomas; and mixed types. A cancer treated as described herein may be of any one of these types and may comprise cells that are precancerous (e.g., benign), malignant, pre-metastatic, metastatic, and/or non-metastatic. A patient who has a malignancy or malignant lesion has a cancer. The present disclosure specifically identifies certain cancers to which its teachings may be particularly relevant.

The term “combination therapy” refers to those situations in which a subject is exposed to two or more therapeutic regimens (e.g., two or more therapeutic agents) to treat a single disease (e.g., a cancer). The two or more regimens/agents may be administered simultaneously or sequentially. When administered simultaneously, a dose of the first agent and a dose of the second agent are administered at about the same time, such that both agents exert an effect on the patient at the same time or, if the first agent is faster- or slower-acting than the second agent, during an overlapping period of time. When administered sequentially, the doses of the first and second agents are separated in time, such that they may or may not exert an effect on the patient at the same time. For example, the first and second agents may be given within the same hour or same day, in which case the first agent would likely still be active when the second is administered. Alternatively, a much longer period of time may elapse between administration of the first and second agents, such that the first agent is no longer active when the second is administered (e.g., all doses of a first regimen are administered prior to administration of any dose(s) of a second regimen by the same or a different route of administration, as may occur in treating a refractory cancer). For clarity, combination therapy does not require that two agents be administered together in a single composition or at the same time, although in some embodiments, two or more agents may be administered within the same period of time (e.g., within the same hour, day, week, or month).

One of ordinary skill in the art will appreciate that the term “dosage form” may be used to refer to a physically discrete unit of an active agent (e.g., a therapeutic or diagnostic agent) for administration to a patient. Typically, each such unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or agent administered to a particular patient is determined by one or more attending physicians and may involve administration of multiple dosage forms.

One of ordinary skill in the art will appreciate that the term “dosing regimen” may be used to refer to a set of unit doses (typically more than one) that are administered individually to a patient, separated by equal or unequal periods of time. A given therapeutic agent typically has a recommended dosing regimen, which may involve one or more doses, each of which may contain the same unit dose amount or differing amounts. In some embodiments, a dosing regimen comprises a first dose in a first dose amount, followed by one or more additional doses in a second dose amount that is different from the first dose amount. In some embodiments, a dosing regimen is correlated with a desired or beneficial outcome when administered across a relevant population (i.e., the regimen is a therapeutic dosing regimen).

As used herein, an “effective amount” of an active agent refers to an amount that produces or is expected to produce the desired effect for which it is administered. The effective amount will vary depending on factors such as the desired biological endpoint, the pharmacokinetics of the active agent administered, the condition being treated, the mode of administration, and characteristics of the patient, as discussed further below and recognized in the art. The term can be applied to therapeutic and prophylactic methods. For example, a therapeutically effective amount is one that reduces the incidence and/or severity of one or more signs or symptoms of the disease. For example, in treating a cancer, an effective amount may reduce the tumor burden, stop tumor growth, inhibit metastasis, or prolong patient survival. One of ordinary skill in the art will appreciate that the term does not in fact require successful treatment be achieved in any particular individual. Rather, a therapeutically effective amount is that amount that provides a particular desired pharmacological response in a significant number of patients when administered to patients in need of such treatment. In some embodiments, reference to a therapeutically effective amount may be a reference to an amount administered or an amount measured in one or more specific tissues (e.g., a tissue affected by the disease) or fluids (e.g., blood, saliva, serum, sweat, tears, urine, etc.). Effective amounts may be formulated and/or administered in a single dose or in a plurality of doses, for example, as part of a dosing regimen.

The term “patient” and “subject” are used interchangeably herein and refer to any organism that is or may be subjected to the administration of an active agent described herein. The administration may be administered for, e.g., experimental, diagnostic, prophylactic, and/or therapeutic purposes. Typical patients include animals (e.g., mammals such as mice, rats, rabbits, non-human primates, and humans; domesticated animals, such as dogs and cats; and livestock or any other animal of agricultural or commercial value). A patient may be suffering from or be susceptible to (i.e., have a higher than average risk of developing) a disease described herein and may display one or more signs or symptoms thereof.

The term “pharmaceutically acceptable,” when applied to a carrier used to formulate a composition disclosed herein (e.g., a pharmaceutical composition), means a carrier that is compatible with the other ingredients of the composition and not deleterious to a patient (e.g., it is non-toxic in the amount required and/or administered (e.g., in a unit dosage form)).

The term “pharmaceutically acceptable,” when applied to a salt, solvate, stereoisomer, tautomer, or isotopic form of an active agent described herein, refers to a salt, solvate, stereoisomer, tautomer, or isotopic form that is, within the scope of sound medical judgment, suitable for use in contact with the tissues of humans (e.g., patients) and lower animals (including, but not limited to, mice and rats used in laboratory studies) without unacceptable toxicity, irritation, allergic response and the like, and that can be used in a manner commensurate with a reasonable benefit/risk ratio. Many pharmaceutically acceptable salts are well known in the art. Pharmaceutically acceptable salts of the active agent s described herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, MALAT1e, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

As used herein, a “reference” refers to a standard or control relative to which a comparison is performed. For example, an agent, patient, population, sample, sequence, or value of interest is compared with a reference agent, patient, population, sample, sequence or value. The reference can be analyzed or determined substantially simultaneously with the analysis or determination of the item of interest or it may constitute a historical standard or control, determined at an earlier point in time and optionally embodied in a tangible medium. One of ordinary skill in the art is well trained in selecting appropriate references, which are typically determined or characterized under conditions that are comparable to those encountered by the item of interest. One of ordinary skill in the art will appreciate when sufficient similarities are present to justify reliance on and/or comparison to a particular possible reference as a standard or control.

As used herein, a “response” to treatment is any beneficial alteration in a patient's condition that results from, or that correlates with, treatment. The alteration may be stabilization of the condition (e.g., inhibition of deterioration that would have taken place in the absence of the treatment), amelioration of, delay of onset of, and/or reduction in frequency of one or more signs or symptoms of the condition, improvement in the prospects for cure of the condition, greater survival time, etc. A response may be a patient's response or a tumor's response.

As used herein, the terms “treatment,” “treat,” and “treating” refer to reversing, alleviating, delaying the onset of, and/or inhibiting the progress of a “pathological condition” (e.g., a disease, such as cancer) described herein. In some embodiments, “treatment,” “treat,” and “treating” require that signs or symptoms of the disease have developed or have been observed. In other embodiments, treatment may be administered in the absence of signs or symptoms of the disease or condition (e.g., in light of a history of symptoms and/or in light of genetic or other susceptibility factors). Treatment may also be continued after symptoms have resolved, for example, to delay or inhibit recurrence.

As the invention relates to compositions and methods for treating patients who have cancer, the terms “active agent,” “anti-cancer agent,” “pharmaceutical agent,” and “therapeutic agent” are used interchangeably (unless the context clearly indicates otherwise). The CDK7 inhibitors and additional active agents described herein are understood to be active, anti-cancer, pharmaceutical, or therapeutic agents. In keeping with convention, in any embodiment requiring two agents, one as the “first” agent and to the other as the “second” agent may be referred to to underscore that the first and second agents are distinct from one another. Where three agents are employed, the “third agent” may be referred to. A salt, solvate, stereoisomer, tautomer, or isotopic form of any active agent disclosed herein can be used in any embodiment described herein.

Relative amounts of the active agent/ingredient, the pharmaceutically acceptable carrier(s), and/or any additional ingredients in a pharmaceutical composition of the invention can vary, depending upon the identity, size, and/or condition of the subject treated and further depending upon the route by which the composition is to be administered and the disease to be treated. By way of example, the composition may comprise between about 0.1% and 99.9% (w/w or w/v) of an active agent/ingredient.

Pharmaceutically acceptable carriers useful in the manufacture of the pharmaceutical compositions described herein are well known in the art of pharmaceutical formulation and include inert diluents, dispersing and/or granulating agents, surface active agents and/or emulsifiers, disintegrating agents, binding agents, preservatives, buffering agents, lubricating agents, and/or oils. Pharmaceutically acceptable carriers useful in the manufacture of the pharmaceutical compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Pharmaceutical compositions used as described herein may be administered orally. Such orally acceptable dosage forms may be solid (e.g., a capsule, tablet, sachet, powder, granule, and orally dispersible film) or liquid (e.g., an ampoule, semi-solid, syrup, suspension, or solution (e.g., aqueous suspensions or dispersions and solutions). In the case of tablets, carriers commonly used include lactose and corn starch. Lubricating agents, such as magnesium stearate, can also be included. In the case of capsules, useful diluents include lactose and dried cornstarch. When aqueous suspensions are formulated, the active agent/ingredient can be combined with emulsifying and suspending agents. In any oral formulation, sweetening, flavoring or coloring agents may also be added. In any of the various embodiments described herein, an oral formulation can be formulated for immediate release or sustained/delayed release and may be coated or uncoated. A provided composition can also be micro-encapsulated.

Compositions suitable for buccal or sublingual administration include tablets, lozenges, and pastilles. Formulations can also be prepared for subcutaneous, intravenous, intramuscular, intraocular, intravitreal, intra-articular, intra-synovial, intrasternal, intrathecal, intrahepatic, intraperitoneal intralesional and by intracranial injection or infusion techniques. Preferably, the compositions are administered orally, subcutaneously, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

Although the descriptions of pharmaceutical compositions provided herein are principally directed to pharmaceutical compositions which are suitable for administration to humans, it will be understood by one of ordinary skill in the art that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification.

Active agents described herein are typically formulated in dosage unit form, e.g., single unit dosage form, for ease of administration and uniformity of dosage. The specific therapeutically or prophylactically effective dose level for any particular subject or organism will depend upon a variety of factors including the disease being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

The amount of an active agent required to achieve an optimum clinical outcome can vary from subject to subject, depending, for example, on species, age, and general condition of a subject, severity of the side effects, cancer to be treated, identity of the particular active agent(s) to be administered, and mode of administration. The desired dosage can be delivered two or three times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

In certain embodiments, an effective amount of an active agent for administration one or more times a day (e.g., once) to a 70 kg adult human may comprise about 1-100 mg, about 1-50 mg, about 1-35 mg (e.g., about 1-5, 1-10, 1-15, 1-20, 1-25, or 1-30 mg), about 2-20 mg, about 3-15 mg or about 10-30 mg (e.g., 10-20 or 10-25 mg). Here, and wherever ranges are referenced, the end points are included. The dosages provided in this disclosure can be scaled for patients of differing weights or body surface and may be expressed per m² of the patient's body surface. In certain embodiments, compositions of the invention may be administered once per day. The dosage of an active agent can be about 1-100 mg, about 1-50 mg, about 1-25 mg, about 2-20 mg, about 5-15 mg, about 10-15 mg, or about 13-14 mg. In certain embodiments, a composition of the invention may be administered twice per day. In some embodiments, the dosage of an active agent for each administration is about 0.5 mg to about 50 mg, about 0.5 mg to about 25 mg, about 0.5 mg to about 1 mg, about 1 mg to about 10 mg, about 1 mg to about 5 mg, about 3 mg to about 5 mg, or about 4 mg to about 5 mg.

The elements and method steps described herein can be used in any combination whether explicitly described or not.

All combinations of method steps as used herein can be performed in any order, unless otherwise specified or clearly implied to the contrary by the context in which the referenced combination is made.

As used herein, the singular forms “a,” “an,” and “the” include plural referents unless the content clearly dictates otherwise.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

All patents, patent publications, and peer-reviewed publications (i.e., “references”) cited herein are expressly incorporated by reference to the same extent as if each individual reference were specifically and individually indicated as being incorporated by reference. In case of conflict between the present disclosure and the incorporated references, the present disclosure controls.

It is understood that the invention is not confined to the particular construction and arrangement of parts herein illustrated and described, but embraces such modified forms thereof as come within the scope of the claims.

### EXAMPLES

### Example 1: CDK7 is a Novel Therapeutic Vulnerability in Fibrolamellar Carcinoma

### Summary

Fibrolamellar Carcinoma (FLC) is a rare and deadly cancer that arises in young patients with otherwise healthy livers. Unfortunately for patients with advanced or recurrent disease that cannot be treated with surgery, only 30-45% of patients survive to 5 years with current treatment options. There is a known cancer-causing mutation found in FLC tumors which results from a fusion of two individual genes, heat shock protein 40 (DNAJB1) and protein kinase A (PRKACA). However, it remains unclear how this DNAJB1-PRKACA fusion causes cancer. Patient tumor samples were used to identify abnormal cell function caused by the DNAJB1-PRKACA fusion, with the goal to exploit these abnormalities for new treatment options. It was found that these tumors manifest irregular cyclin dependent kinase 7 (CDK7) activity and rely on CDK7 for growth. By using drug inhibitors of CDK7 this irregular activity can be disrupted and cancer cell death can be induced. Taken together, this suggests CDK7 is a good target for future treatment in FLC.

### Overview

Fibrolamellar carcinoma (FLC) is a rare and lethal cancer that afflicts young individuals. The tumor arises in the background of a healthy liver, and patients typically present with advanced cancer at the time of diagnosis. Unfortunately, for these patients with advanced or recurrent cancer, no proven systemic therapies exist resulting in only 30-45% of patients surviving to 5 years. Investigations into the molecular underpinning of FLC have revealed a unique gene fusion between heat shock protein 40 (DNAJB1) and the catalytic subunit alpha of protein kinase A (PRKACA), leading to the formation of an oncoprotein (DNAJ-PKAc) that retains kinase activity and is a proven tumor-causing event in FLC. While the precise signaling cascade downstream of DNAJ-PKAc remains unknown, recent work has demonstrated that the fusion oncoprotein causes aberrant remodeling of the epigenetic landscape and emergence of FLC specific super enhancers. RNA Pol II is known to be highly enriched at super enhancers. Consequently, human samples of FLC (versus paired adjacent liver) were evaluated for aberrant RNA Pol II phosphorylation and found increased serine-5 phosphorylation; serine-5 is the molecular target of cyclin dependent kinase 7 (CDK7). To investigate the role of CDK7, an FLC cell line was engineered by introducing the DNAJB1-PRKACA oncogene rearrangement into human hepatocellular cells and identified heightened CDK7 activation (RNA Pol II serine-5 phosphorylation) in the DNAJB1-PRKACA expressing FLC cells. In turn, targeting CDK7 with selective inhibitors suppressed expression of FLC-specific super enhancer driven genes and induced cell death in several patient-derived models of FLC, with minimal toxicity to normal liver. Finally, selectively targeting aberrant RNA Pol II activity with combination CDK7 and CDK9 inhibition revealed a potent combination therapy. Collectively, this work uncovers a novel candidate therapeutic vulnerability in FLC.

### Introduction

Fibrolamellar carcinoma (FLC) is a rare and lethal cancer that afflicts young individuals, and accounts for approximately 1% of liver cancers (Eggert et al. 2013, O'Neill et al. 2021, Dinh et al. 2022, Simon 2023). The tumor arises in the background of a normal liver, and patients typically present with advanced cancer at the time of diagnosis (Kastenhuber et al. 2017, Dinh et al. 2022); this is due to the absence of known risk factors or tumor markers that can precipitate screening or prevention (Eggert et al. 2013, Honeyman et al. 2014) (Patt et al. 2003, Abou-Alfa et al. 2020, El Dika et al. 2020). While surgery offers the best chance for long-term survival, most patients experience cancer recurrence after surgical resection (Eggert et al. 2013, Kastenhuber et al. 2017). Unfortunately, for these patients with advanced or recurrent cancer, no proven systemic therapies exist, and only 30-45% of patients survive to 5 years (Eggert et al. 2013, Honeyman et al. 2014). When considering that the peak age of diagnosis is 22 years (Dinh et al. 2022), the substantial impact on life-years lost underscores the devastating nature of this cancer. Thus, there remains a critical need to improve outcomes for patients with FLC through systemic treatment.

Undoubtedly, the research landscape in FLC changed after the 2014 pioneering study that revealed a unique gene fusion created by a ˜400 kbp deletion between heat shock protein 40 (DNAJB1) and the catalytic subunit alpha of protein kinase A (PRKACA) (Honeyman et al. 2014). The DNAJB1-PRKACA gene fusion is the proven driver mutation in the vast majority of human FLC, and the resultant chimeric protein (DNAJ-PKAc) is only present in tumor cells indicating a somatic event (Oikawa et al. 2015, Kastenhuber et al. 2017, Dinh et al. 2020). Since its initial discovery, there has been inconclusive evidence of how DNAJ-PKAc drives cellular changes to promote neoplastic transformation and progression of FLC. Concordantly, there have been concerted efforts to understand how DNAJB1-PRKACA drives tumor development and mechanisms of treatment resistance so that new and effective therapies can be identified (Turnham et al. 2019, Dinh et al. 2020). For instance, the chimeric protein demonstrates enhanced PKA activity and aberrant localization compared to native PKA (Honeyman et al. 2014, Shirani et al. 2024), but inhibiting PKA has proven challenging in FLC given the critical nature of PKA in normally functioning cells (i.e., no therapeutic window) (Neumayer et al. 2023). While the precise signaling cascade downstream of DNAJ-PKAc remains unknown, recent work has demonstrated that the fusion oncoprotein causes aberrant remodeling of the epigenetic landscape and emergence of FLC specific super enhancers (Dinh et al. 2020).

Super enhancers within cancer cells are responsible for high levels of expression of genes that are essential for cell identity and survival (Hnisz et al. 2013, Chipumuro et al. 2014). These critical genes associated with super enhancers control cell state and cancer hallmark qualities (Hnisz et al. 2013). Notably, the profile of super enhancer driven genes in FLC uncovered key molecular targets that are uniquely overexpressed in FLC such as SLC16A14 and LINC00473among others (Dinh et al. 2020, Ma et al. 2024). These FLC-specific genes can serve as markers for DNAJ-PKAc oncoprotein activity, but a critical gap exists in understanding how the tumor-driver mutation (DNAJB1-PRKACA) transmits signals to ultimately promote super enhancer driven gene expression in FLC.

RNA Polymerase II (along with other cofactors and chromatin regulators) is known to be highly enriched at super enhancers—and can transcribe enhancer RNA that may contribute to enhancer function (Hnisz et al. 2013). Human samples of FLC (and paired adjacent liver) were evaluated for heightened RNA Polymerase II (RNA Pol II) phosphorylation, which is an indicator of increased RNA Pol II activity, and found increased cyclin dependent kinase 7 (CDK7) phosphorylation and serine-5 phosphorylation. Serine-5 is the molecular target of CDK7; the presence of phosphorylated CDK7 and serine-5 indicates aberrantly activated CDK7 in tumors harboring DNAJ-PKAc. To confirm, an FLC cell line was engineered by introducing the DNAJB1-PRKACA oncogene rearrangement into human hepatocellular cells and found clear evidence that the DNAJB1-PRKACA expressing cells possessed significantly increased CDK7 activity. Further, CDK7 activation promoted RNA Pol II phosphorylation and was responsible for regulating FLC-specific super enhancer driven genes, which aligns with the known function of CDK7 as a regulator of RNA Pol II activity (Larochelle et al. 2006, Fisher 2019). Selectively suppressing CDK7 in patient-derived models of FLC resulted in potent induction of cancer cell death. CDK7 inhibition can also combine synergistically to markedly suppress RNA Pol II phosphorylation and abolish FLC-specific super enhancer driven gene expression. For instance, targeting aberrant RNA Pol II activity with combination CDK7 and CDK9 inhibition revealed a potent synergistic therapy. Collectively, this work uncovers a novel candidate therapeutic vulnerability in FLC.

### Results

### Increased RNA Polymerase II Phosphorylation in Human Samples of FLC

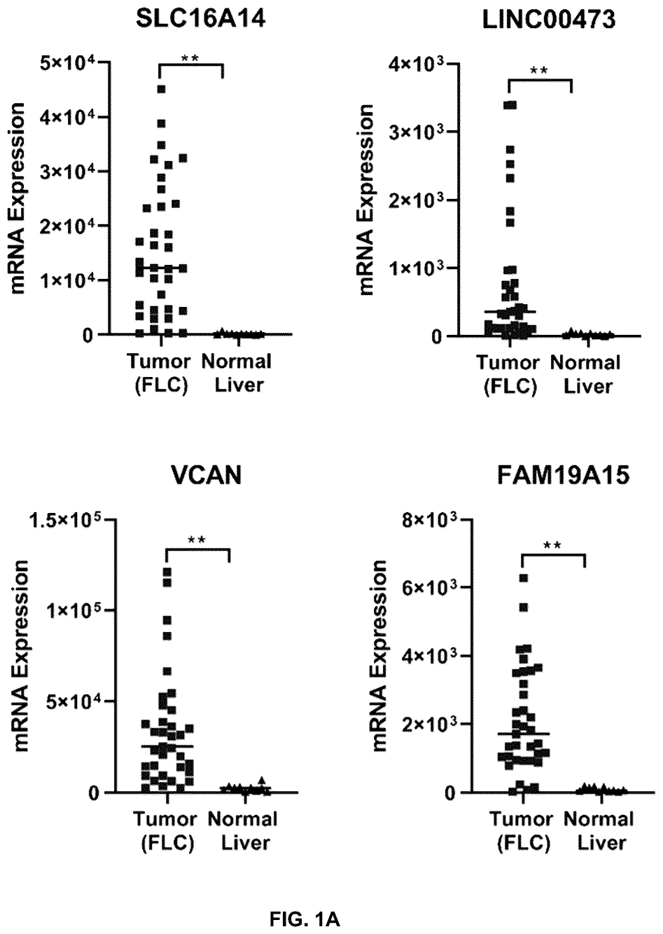

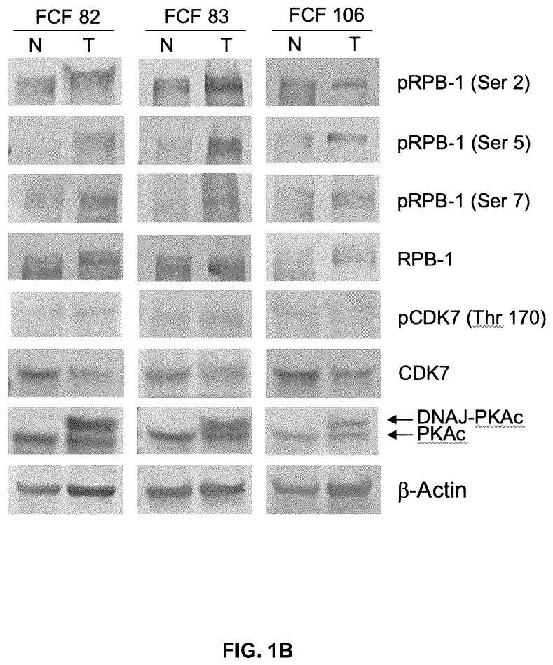

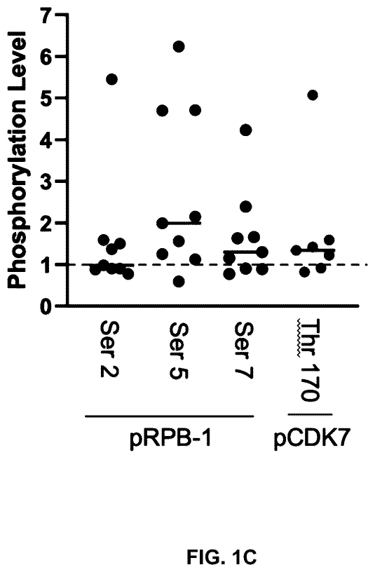

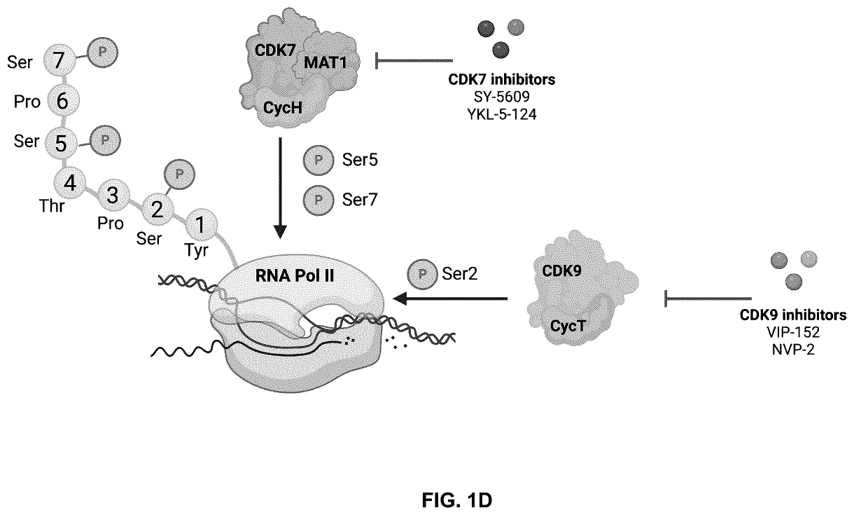

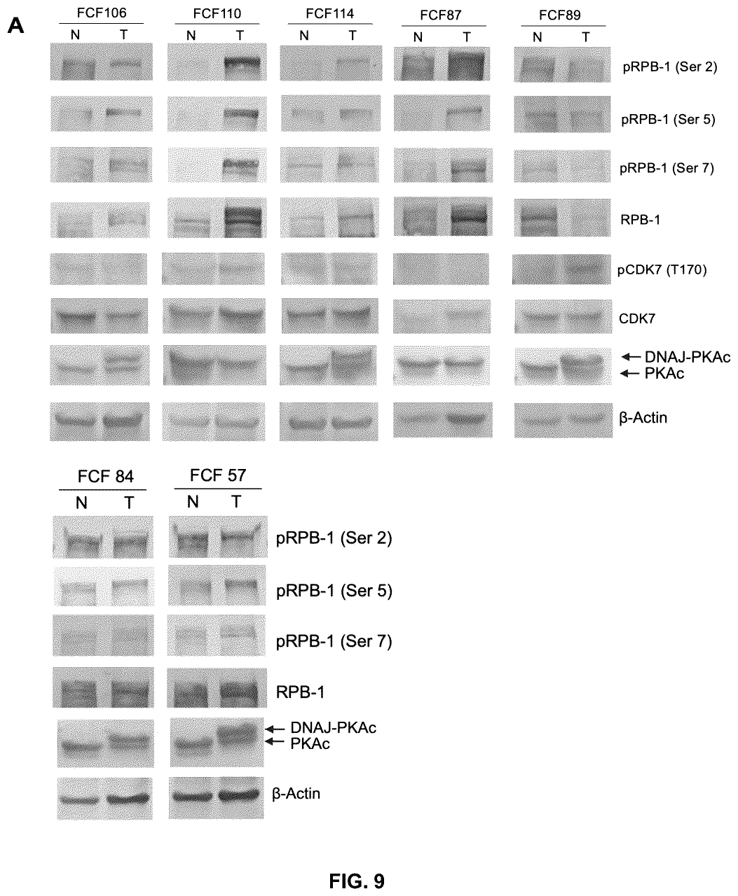

Key insight into the transcriptional changes associated with the DNAJ-PKAc oncoprotein in FLC came from recent work depicting the super enhancer landscape in human tumors of FLC (Dinh et al. 2020). This led to the identification of molecular targets that are overexpressed in FLC, one of which (SLC16A14) is highly specific for FLC and consistently upregulated in tumors compared to normal tissue (FIG. 1A). The seminal work that elaborated super enhancer driven genes utilized a technique that measures nascent RNA, including enhancer RNA transcribed by RNA Pol II (Dinh et al. 2020). RNA Pol II is known to be highly enriched at super enhancers (Hnisz et al. 2013), and to understand what may be driving the aberrant RNA Pol II function, protein was isolated from stored frozen human samples of FLC/paired normal liver and performed immunoblot to assess for RNA Pol II phosphorylation. Increased CDK7 and serine-5 phosphorylation, and to a lesser extent serine-7 phosphorylation, was found in the human tumor tissue versus human adjacent liver (FIG. 1B, FIG. 9). Phosphorylation levels were quantified, and a phosphorylation index was generated for serine-2, serine-5, serine-7 and CDK7 (i.e. pRPB-1/RPB-1, pCDK7/CDK7); the phosphorylation level was then quantified as phosphorylation index (tumor)/phosphorylation index (adjacent). (FIG. 1C). An important kinase involved in RNA Pol II activity is cyclin dependent kinase 7 (CDK7). CDK7 binds cyclin H and MAT1 to form the CDK-activating kinase (CAK) which phosphorylates target proteins at serine and threonine sites (Lolli and Johnson 2005, Peissert et al. 2020). CDK7 modulates transcription by phosphorylating RNA Pol II at specific serine residues in the c-terminal domain (CTD), including serine-5(preferential target), and potentially serine-7 and serine-2, although serine-2 phosphorylation may occur indirectly through regulation of CDK9 (FIG. 1D) (Ebmeier et al. 2017). CDK7 specific inhibitors (SY-5609 and YKL-5-124) and CDK9 specific inhibitors (VIP-152 and NVP-2) that are tested in these examples are shown.

### CDK7 Mediates DNAJB1-PRKACA Driven Super Enhancer Gene Expression

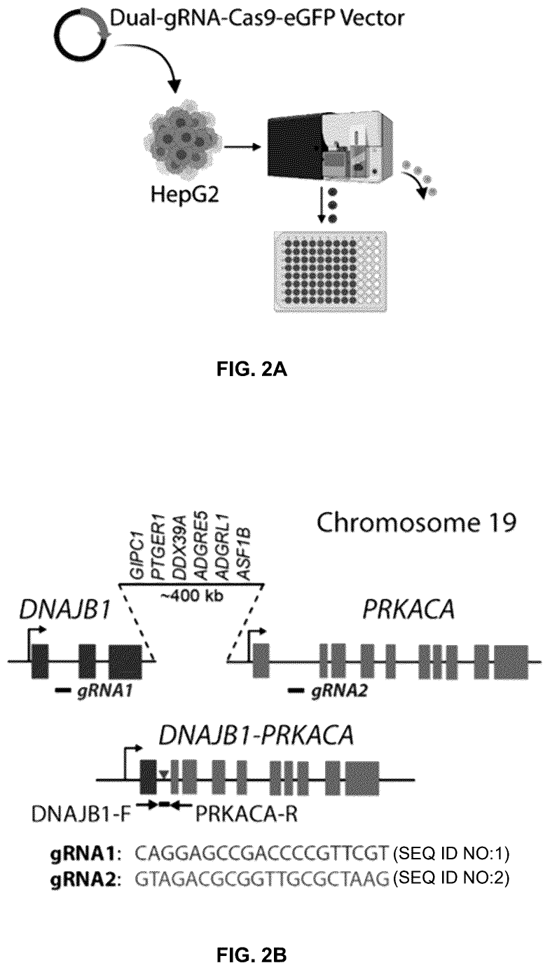

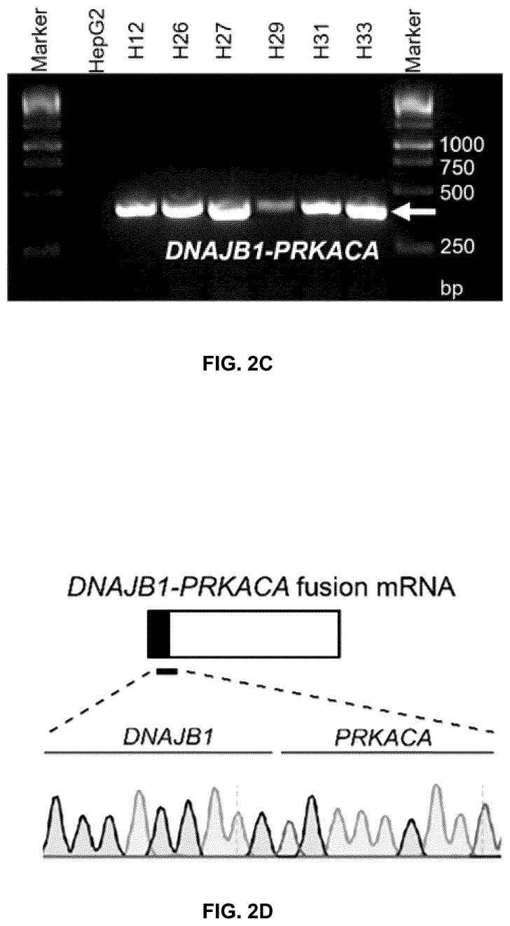

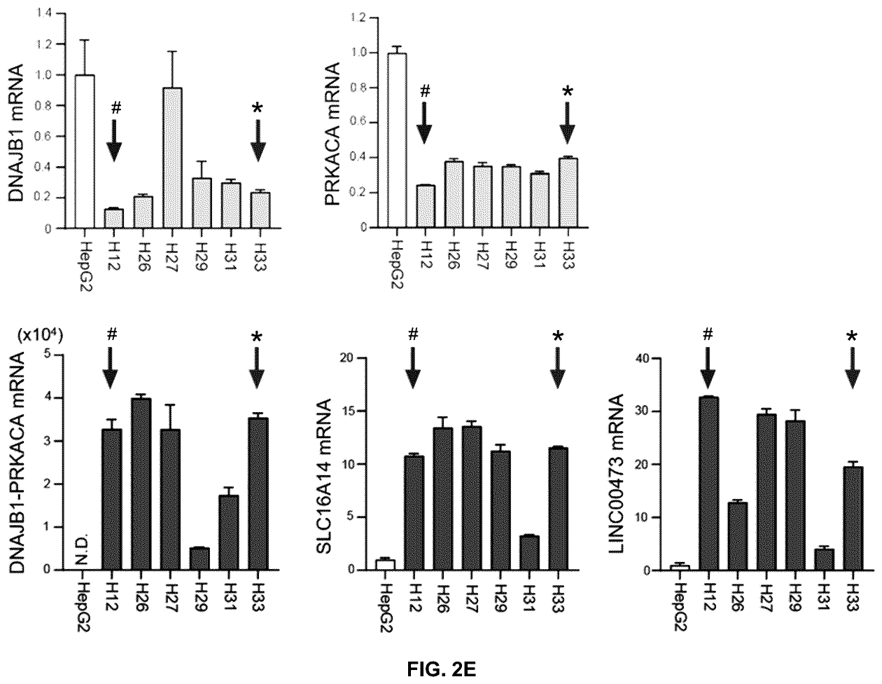

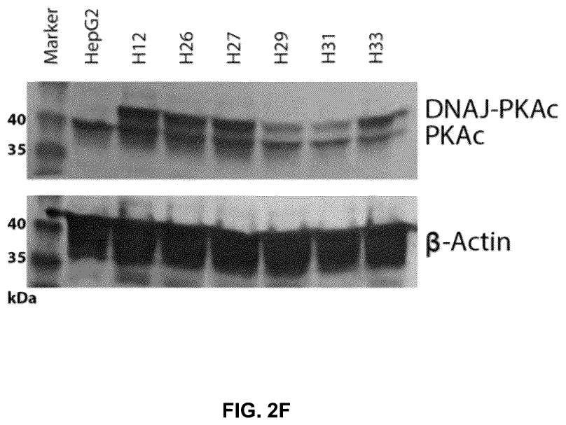

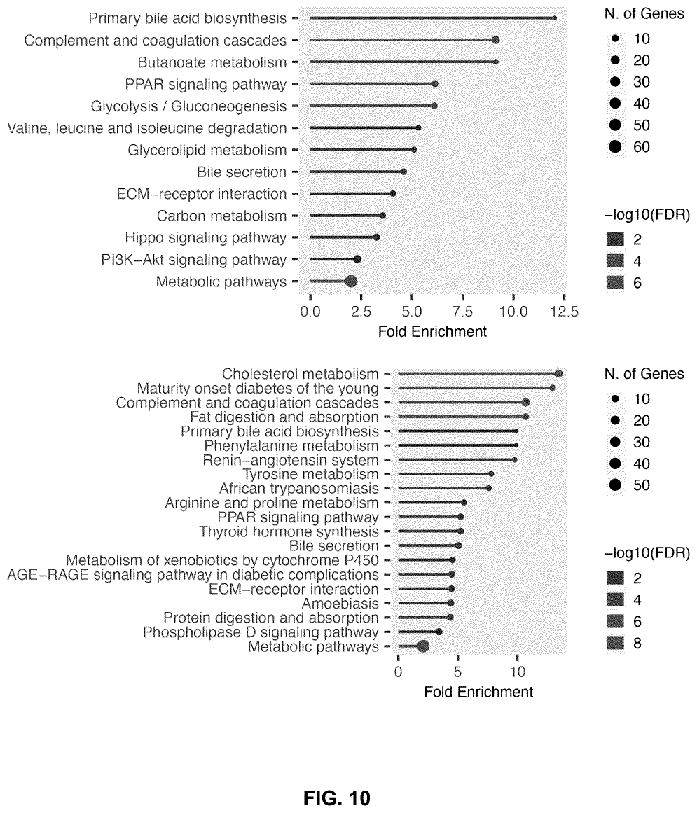

To investigate the role of CDK7 signaling in FLC, the DNAJB1-PRKACA gene fusion was engineered in a hepatoblastoma cell line (HepG2) (Lopez-Terrada et al. 2009), using dual-guide RNA/Cas9 that targets the intronic region between exon 1 and 2 of DNAJB1 and the intronic region between exon 1 and 2 of PRKACA (FIGS. 2A-2B). Genomic DNA PCR and sequencing across the breakpoint established presence of the DNAJB1-PRKACA gene fusion in six separate clones (FIGS. 2C-2D). mRNA expression of DNAJB1-PRKACA was confirmed and sequencing of the mRNA transcripts was performed to ensure development of the expected fusion transcript (FIGS. 2D-2E). Protein analysis demonstrated presence of DNAJ-PKAc and native PKA, which is the expected pattern of protein detection in human FLC (FIG. 2F) (Honeyman et al. 2014). Based on expression levels of native genes (e.g., PRKACA) and FLC-specific markers (e.g., SLC16A14 and LINC00473), clone H33 (HepG2^{DNAJB1-PRKACA}) was selected for subsequent assays (FIG. 2E). Another clone, H12 (HepG2^{DNAJB1-PRKACA}), also met the same rigorous validation metrics and was utilized for confirmatory tests. Finally, KEGG pathway analysis was performed to compare the DNAJB1-PRKACA expressing model system to human FLC and identified strong overlap of enriched pathways (FIG. 10).

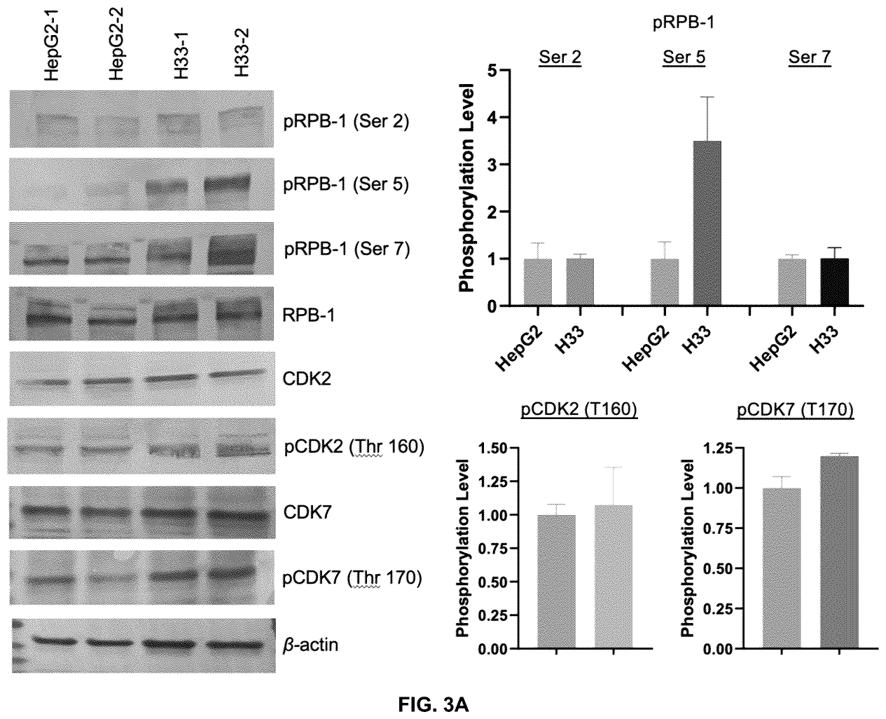

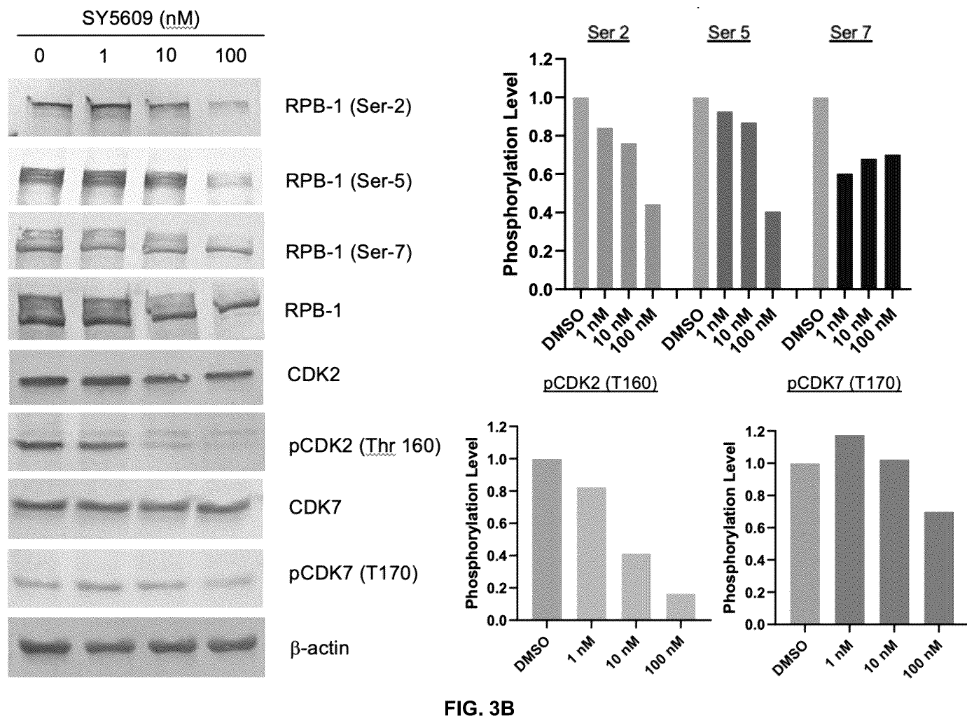

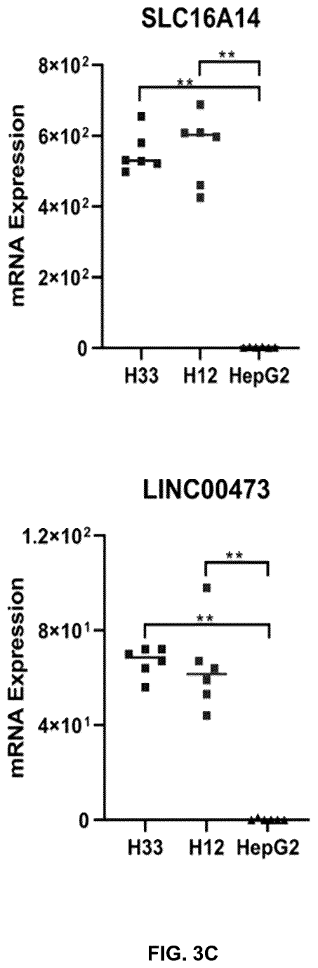

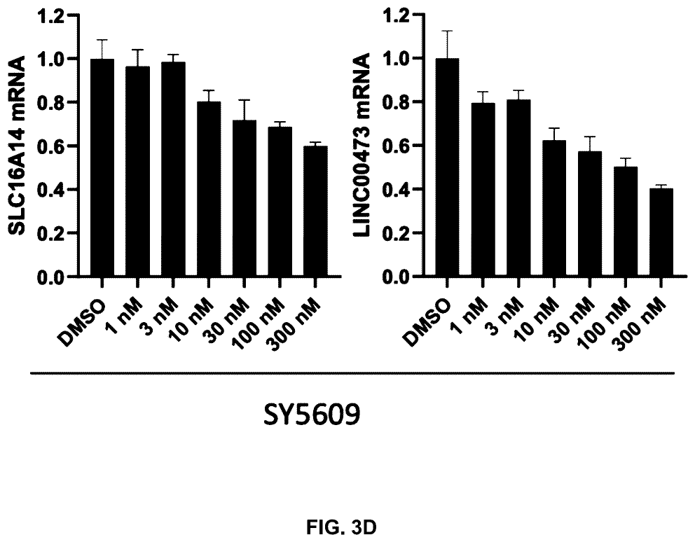

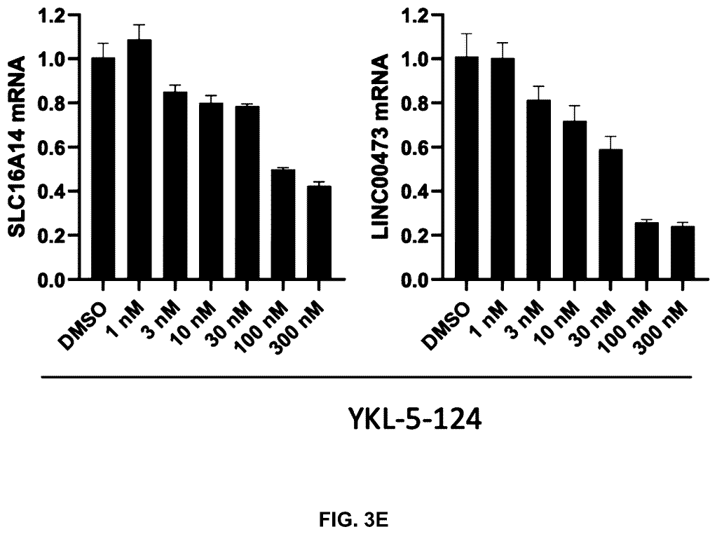

Immunoblotting was performed in the DNAJB1-PRKACA expressing H33 cells and parent HepG2 cells including phosphorylation of RNA Pol II (serine-5, serine-7 and serine-2 of C-terminal domain), CDK2 and CDK7, and found increased levels of phosphorylated serine residues in the CTD of RNA Pol II (FIG. 3A). Serine-5 phosphorylation was the most prominent, substantiating the human sample data. Because of the known function of CDK7 in RNA Pol II phosphorylation, a potent and specific CDK7 inhibitor that is currently in use in clinical trials (SY-5609) (Marineau et al. 2022) was used to evaluate the impact of CDK7 inhibition in the DNAJB1-PRKACA expressing H33 cells. It was examined whether CDK7 modulated the increased phosphorylation of RNA Pol II in DNAJB1-PRKACA expressing H33 cells compared to HepG2 cells. With SY-5609 treatment, clear suppression of phosphorylation of serine-2,-5, and-7 (CTD) of RNA Pol II (FIG. 3B) was identified. These data support the idea that CDK7 mediates these specific DNAJB1-PRKACA driven changes in H33 cells versus HepG2 cells. Interestingly, CDK7 is unique amongst the CDKs in that it is has a dual role as a master regulator of cell cycle (i.e., phosphorylates CDK1, CDK2) and a transcriptional regulator (i.e., phosphorylates RNA Polymerase II) (Lolli and Johnson 2005, Ebmeier et al. 2017, Peissert et al. 2020). Consequently, inhibition of CDK7 with SY-5609 resulted in decreased levels of both phosphorylated (T170) CDK7 and phosphorylated (T160) CDK2 in the DNAJB1-PRKACA expressing H33 cells.

Prior work has shown that in human FLC there is aberrant super enhancer activity due to the DNAJ-PKAc oncoprotein, with consequent increased expression of FLC specific genes including SLC16A14 and LINC00473 (Dinh et al. 2020). This overexpression was identified in the DNAJB1-PRKACA expressing HepG2 cells (H33 and H12 clones (FIG. 3C). Notably, SLC16A14 and LINC00473 are highly expressed in human FLC compared to normal liver and are not elevated in other liver cancers (such as cholangiocarcinoma or HCC) (Dinh et al. 2020). The function of these genes in FLC and the impact of their marked overexpression (compared to paired normal liver) remain unknown but the products of these genes may serve as unique therapeutic vulnerabilities in FLC and is the focus of current investigations (Dinh et al. 2020, Ma et al. 2024). RNA Pol II (along with other cofactors and chromatin regulators) is known to be highly enriched at super enhancers—and can transcribe enhancer RNA that may contribute to enhancer function (Hnisz et al. 2013). Given the importance of CDK7 in facilitating RNA Pol II function (via phosphorylation), and the known role of CDK7 in cancer associated super enhancers (Larochelle et al. 2012, Chipumuro et al. 2014, Christensen et al. 2014, Kwiatkowski et al. 2014), it was tested whether the aberrant CDK7 activation may be driving the expression of these FLC-specific super enhancer associated genes.

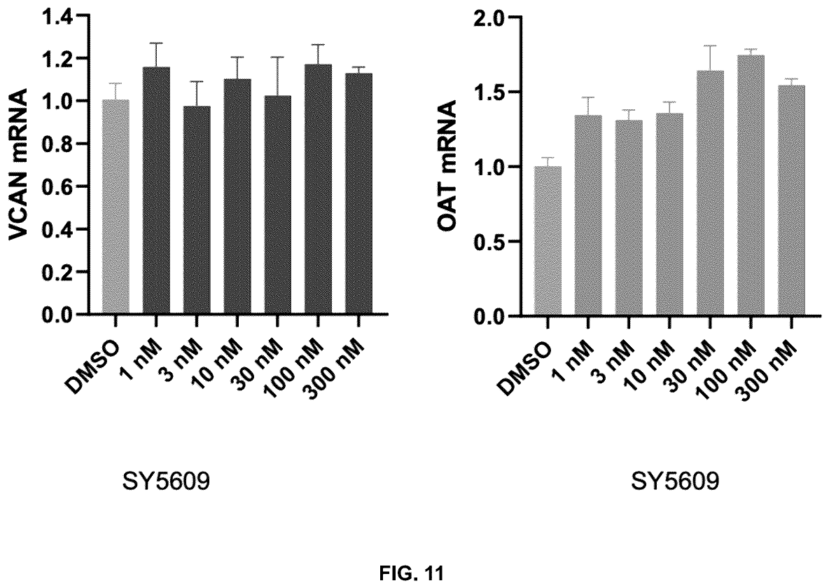

DNAJB1-PRKACA expressing H33 cells were treated with SY-5609 and a substantial downregulation of both SLC16A14 and LINC00473 expression was found (FIG. 3D), suggesting a critical role of CDK7 in promoting super enhancer driven gene expression in FLC. To confirm these results, a separate potent and selective covalent CDK7 inhibitor that irreversibly and covalently binds CDK7 (YKL-5-124) (Olson et al. 2019) was used, and found identical downregulation of SLC16A14 and LINC00473 expression (FIG. 3E). These data demonstrate for the first time that SLC16A14 and LINC00473 are novel CDK7 regulated genes, and that CDK7—which mediates phosphorylation of RNA Pol II in DNAJB1-PRKACA expressing cells—is responsible for regulating expression of FLC-specific super enhancer driven genes. Furthermore, and of potential clinical importance, CDK7 inhibition can be used to target both SLC16A14 and LINC00473 in FLC, which is a key finding given the current inability to inhibit these highly promising targets in FLC (i.e., they are currently not ‘druggable’). In addition, the testing of other FLC-specific genes that have been identified in patient samples such as OAT and VCAN show no decrease in expression in response to SY-5609, suggesting there is a specific mechanism by which CDK7-RNA Pol II inhibition regulates SLC16A14 and LINC00473 in FLC beyond global transcriptional suppression (FIG. 11).

### CDK7 Inhibition Suppresses Cancer Cell Growth in Human Models of FLC

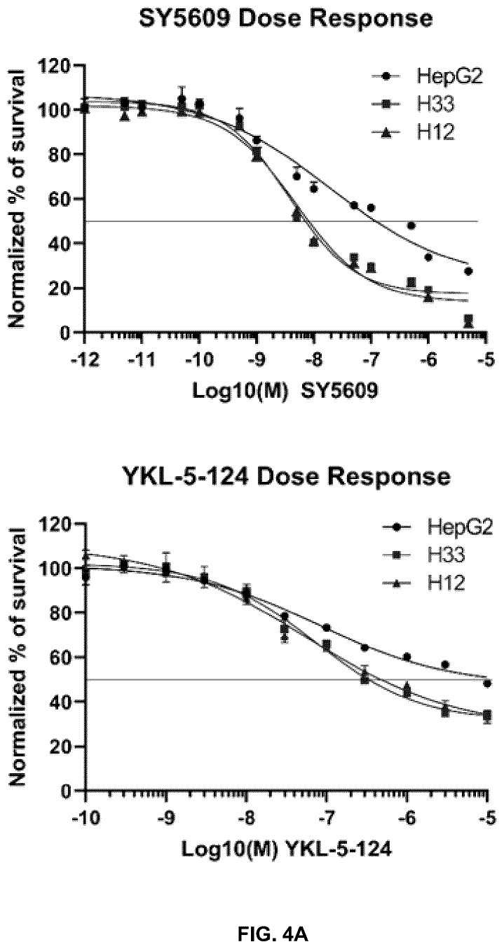

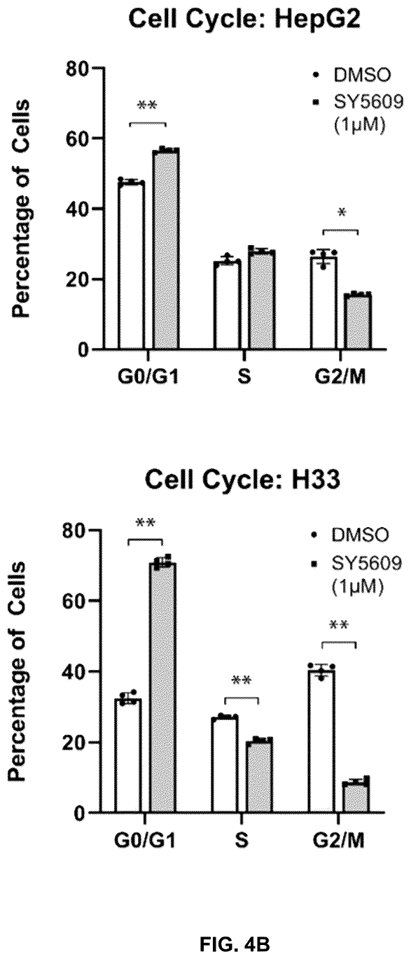

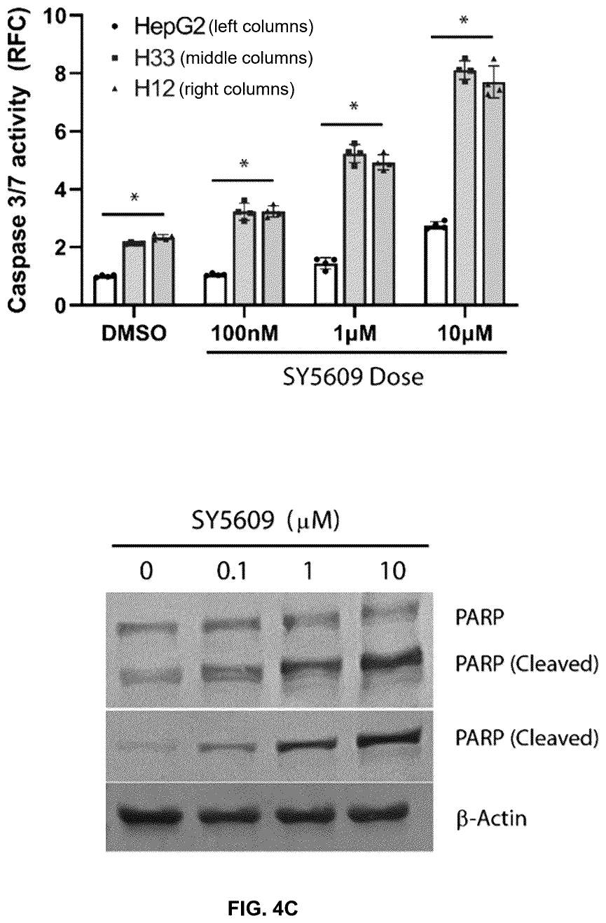

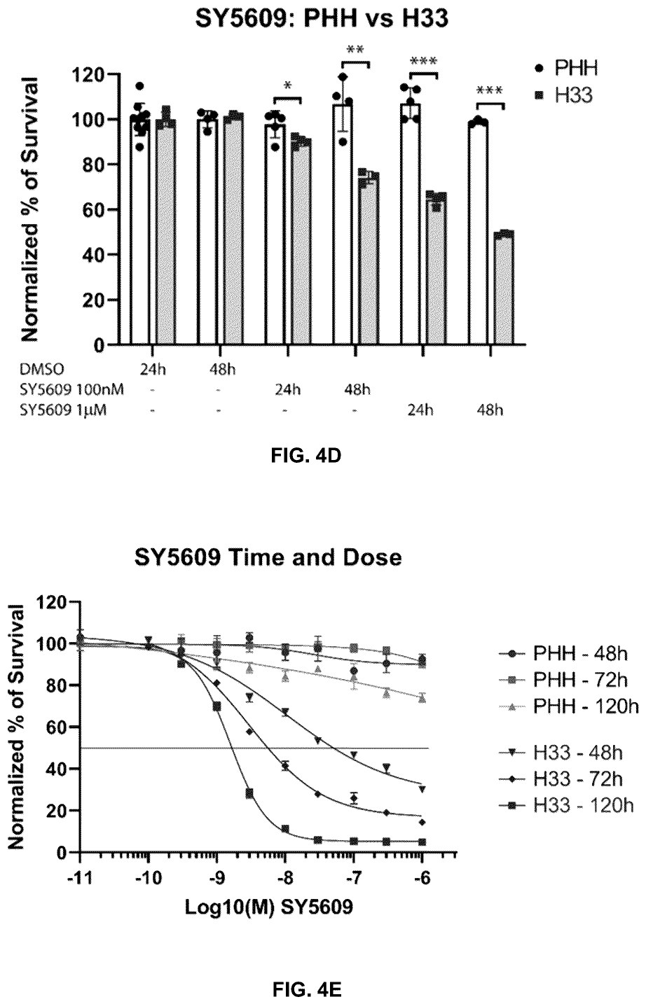

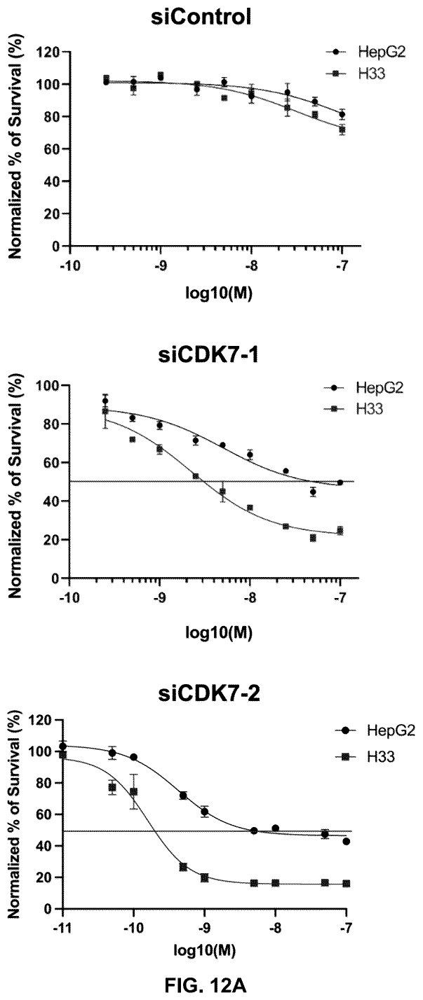

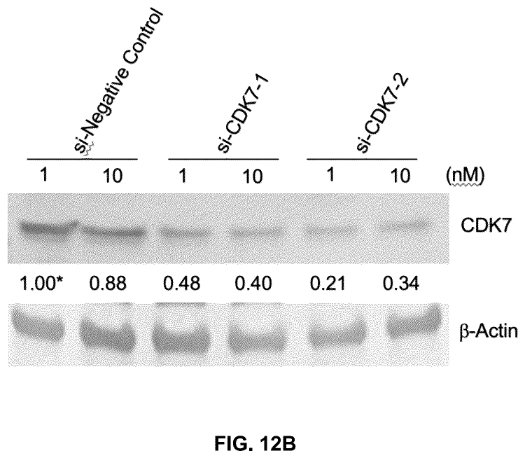

Given the evidence of CDK7 controlling expression of compelling FLC-specific therapeutic targets (i.e., SLC16A14 and LINC00473) (Ma et al. 2024, Dinh et al. 2020), and the known role of CDK7 on RNA Pol II transcriptional regulation (Larochelle et al. 2006, Fisher 2019), it was hypothesized that the impact of CDK7 inhibition on super enhancer-driven transcriptional changes would provide a therapeutic vulnerability in FLC cells. Whether CDK7 inhibition impacted cell proliferation in H33 cells was therefore tested. SY-5609 demonstrated a strong dose-dependent reduction in cell viability, which was confirmed in a separate clone (H12) (FIG. 4A). To ensure that the observed effect was CDK7 dependent, cell viability with YKL-5-124 was assessed. YKL-5-124 treatment resulted in a similar dose-dependent reduction of H33 cell viability (FIG. 4A). As an additional confirmation, siRNA was used against CDK7 and again demonstrated a strong dose-dependent response (FIGS. 12A-12B), thus revealing that targeting CDK7 dramatically impairs cell viability (i.e., a therapeutic vulnerability) in the FLC model of DNAJB1-PRKACA expressing H33 cells.

The marked reduction in cancer cell viability due to CDK7 inhibition may be due to cell cycle arrest, induction of apoptosis, or both. The effect of CDK7 inhibition on cell cycle was first measured. It was found that SY-5609 treatment resulted in a modest effect in the HepG2 cells, with a moderately reduced percentage of cells in G2/M (26.4% DMSO vs 15.5% SY-5609, p=0.0015) and a reciprocal increase in the percent of cells in G0/G1 (47.6% DMSO vs 56.6% SY-5609, p<0.0001). In contrast, CDK7 inhibition in H33 cells resulted in a profound cell cycle shift with most cells in G0/G1 (32.5% DMSO vs 70.9% SY-5609, p<0.0001) and a marked reduction in the percent of cells in G2/M (40.4% DMSO vs 8.7% SY-5609, p<0.0001) (FIG. 4B). The effect on apoptosis with CDK7 inhibition was subsequently examined. SY-5609 treatment of H33 cells demonstrated a significant increase in caspase 3/7 activity compared to HepG2 cells at all treatment doses (SY-5609 100 nM, 1 μM, 10 μM, all p<0.0001), which was confirmed with the separate H12 clone, demonstrating high sensitivity of DNAJB1-PRKACA expressing cells to CDK7 inhibition. The significant increase in apoptosis through detection of cleaved PARP1 was again validated, which displayed a clear dose-dependent increase in cleaved protein with SY-5609 treatment (FIG. 4C). Together, this data shows that CDK7 is a critical component of cell proliferation/viability in DNAJB1-PRKACA expressing cells, and likewise, CDK7 may represent a key therapeutic vulnerability to exploit in FLC.

A necessary component of any potential therapy is the identification of a true therapeutic window. Because toxicity may limit translational potential (i.e., therapy tested must be tolerated), the effect of CDK7 inhibition in human liver cells was assessed. Human hepatocytes were isolated immediately from fresh liver (transplant donor specimen) and examined the impact of SY-5609 at two separate doses (100 nM and 1 μM) for 24- and 48-hours (FIG. 4D). While the DNAJB1-PRKACA expressing H33 cells demonstrated dose and time-dependent sensitivity to SY-5609 treatment, there was no change in cell viability in the primary human hepatocyte (PHH) liver cells compared to DMSO control, confirming the lack of toxicity to normal liver cells at the doses tested. The findings with separate dose-response experiments in the human hepatocytes was confirmed at 48-, 72-, and 120-hours treatment with SY-5609 (FIG. 4E), to ensure minimal toxicity even with increased duration of exposure. Notably, with increasing time, the LC₅₀ (IC₅₀) value of SY-5609 decreased in the H33 cells (IC₅₀ shifts to the left: 7.97E-8 M, 8.67E-9 M and 1.71E-9 M at 48-, 72- and 120-hours respectively), indicating both a dose and time-dependent cell death. Meanwhile, there was minimal impact on the human hepatocytes, even out to 120-hours treatment duration. A therapeutic vulnerability in DNAJB1-PRKACA expressing H33 cells was therefore identified that was not present in the human fresh liver tissue (i.e., no cytotoxicity), indicating an excellent therapeutic window/index for CDK7 inhibition.

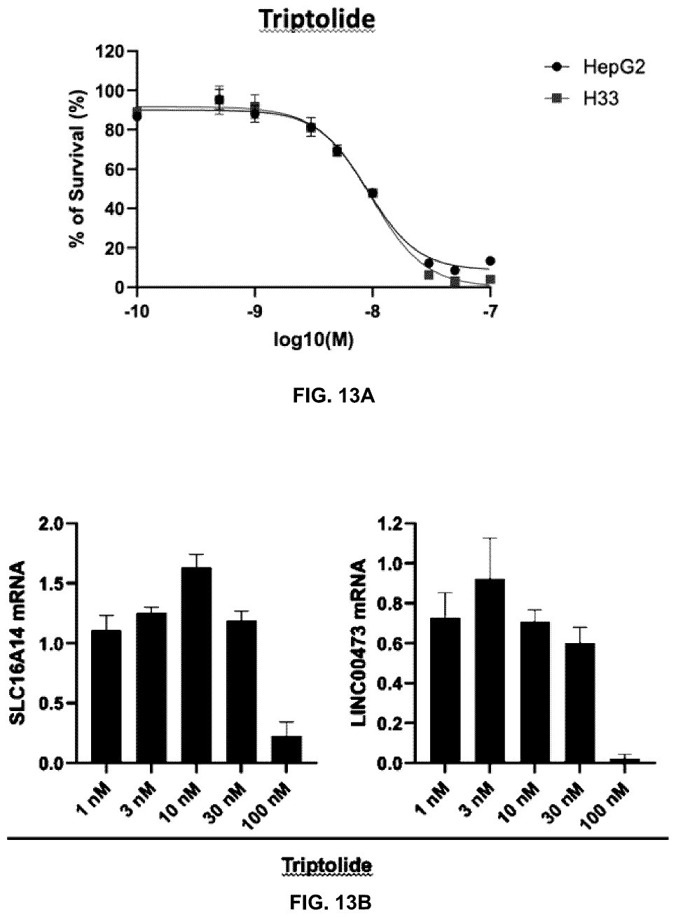

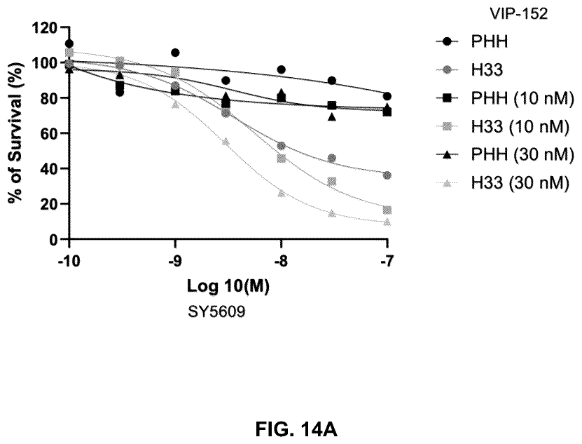

To validate that these changes were due to CDK7 inhibition and not simply suppression of RNA Pol II (or global transcriptional suppression) Triptolide was tested, an inhibitor of the translocase activity of TFIIH (Wang et al. 2011). While a dose dependent decrease in cell survival was not observed, there was no difference between survival in HepG2 and H33 cells indicating no unique sensitivity in DNAJB1-PRKACA expressing cells (FIG. 13A). Further, the expression of the FLC specific genes SLC16A14 and LINC00473 was assessed in H33 cells treated with triptolide and only observed a statistically significant decrease in expression at high doses, without a dose-dependent linear reduction as was seen with CDK7 inhibition (i.e., ‘shutting off’ phenomena, FIG. 13B). Taken together, this suggests that DNAJB1-PRKACA expressing H33 cells are uniquely sensitive to CDK7 inhibition that is not seen with inhibition of global transcription via generalized RNA Pol II suppression.

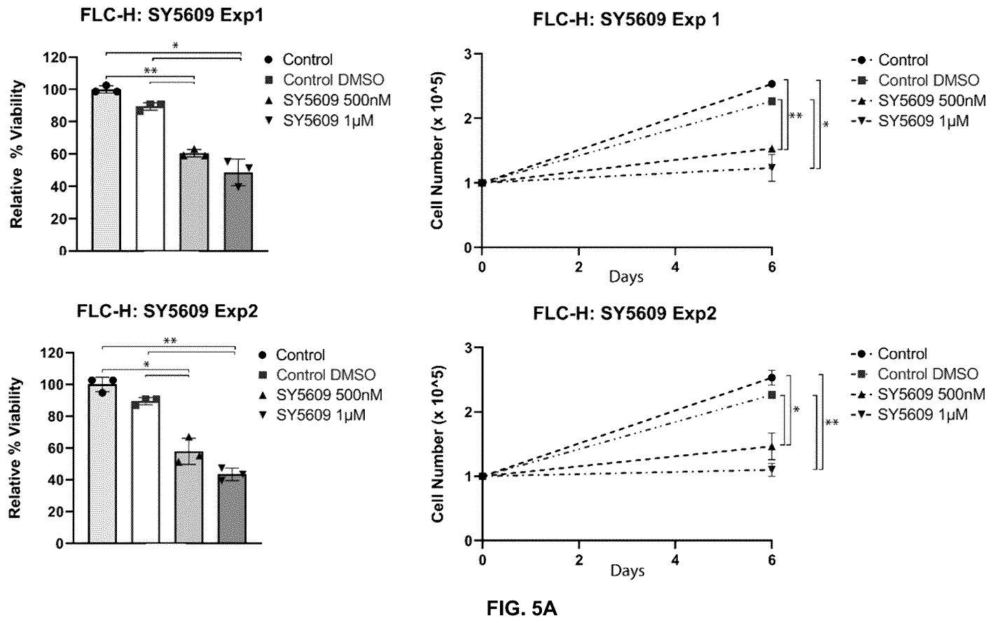

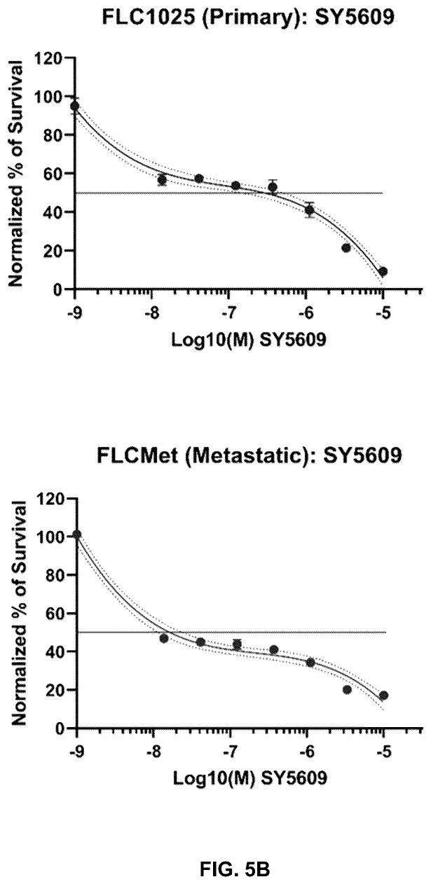

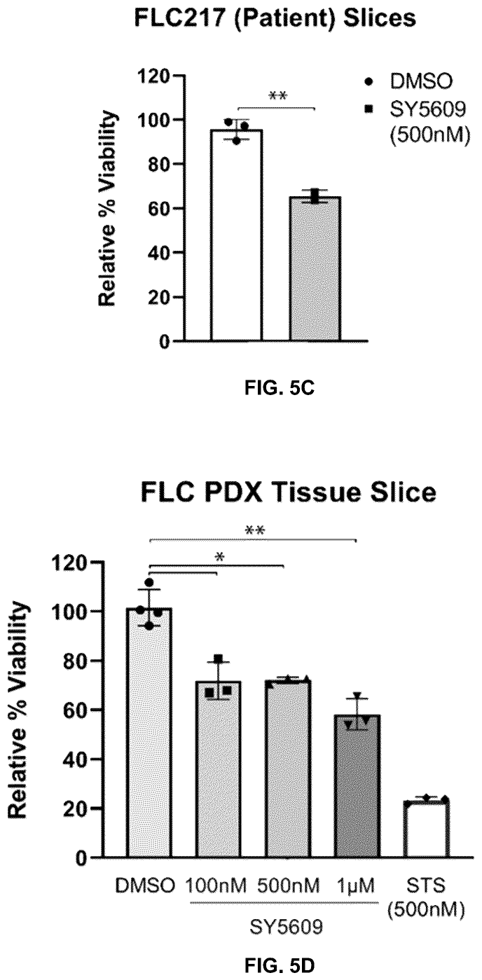

To better understand the clinical relevance of the results, CDK7 inhibition was tested independently in five separate patient-derived models. Given the extraordinarily rare nature of FLC, patient-derived models are typically limited. Using a patient-derived FLC cell line that has been well characterized (FLC-H) (Oikawa et al. 2015, Dinh et al. 2020), suppression of cell growth after SY-5609 treatment compared to control treated cells was found, with a greater than 50% reduction in cell viability (SY-5609 1 μM, p<0.01) (FIG. 5A). Using two distinct patient-derived FLC cell lines (FLC1025 derived from primary tumor, FLCMet derived from metastatic disease), SY-5609-induced dose-dependent reduction in cell viability was identified, confirming the findings from the H33 cells and the FLC-H cell line (FIG. 5B). The effect of SY-5609 in tissue slices derived from human FLC (FIG. 5C) and from a separate set of tissue slices derived from a patient-derived xenograft of FLC (FIG. 5D) was next tested. This demonstrated a similar reduction in cell viability compared to the patient-derived cell lines, which confirmed the efficacy of CDK7 inhibition.

Dual Targeting of RNA Polymerase II with CDK7 Inhibitor Backbone as a Therapeutic Strategy

Given the recalcitrance of FLC to systemic therapy, single-agent therapy may be unlikely to overcome this treatment resistance, and combination therapy may be necessary. Partners with CDK7 inhibitors were therefore considered. CDK7 inhibition caused suppressed super enhancer driven gene expression, likely through its impact on RNA Pol II ser-5 phosphorylation. Meanwhile, CDK9 is responsible for phosphorylating RNA Pol II ser-2 and enables RNA Pol II to transition to effective transcriptional elongation after CDK7-mediated release of RNA Pol II from the pre-initiation complex (Eick and Geyer 2013, Vervoort et al. 2021). CDK7 has been reported to potentially modulate CDK9 activity, but the extent that suppression of CDK7 activity can abolish kinase activity of CDK9 is unclear (Larochelle et al. 2012, Ebmeier et al. 2017). Therefore, in consideration for synergistic partners, CDK7 inhibition was evaluated in combination with CDK9 inhibition which should dual-combine to suppress the aberrant RNA Pol II activity seen in DNAJB1-PRKACA expressing cells (i.e., human tumors and H33 cells).

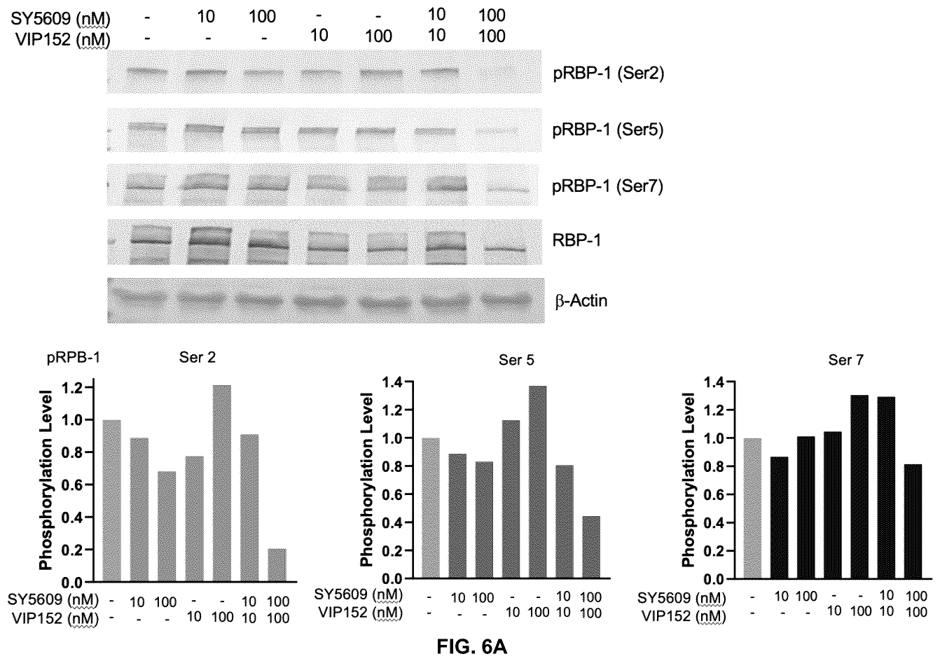

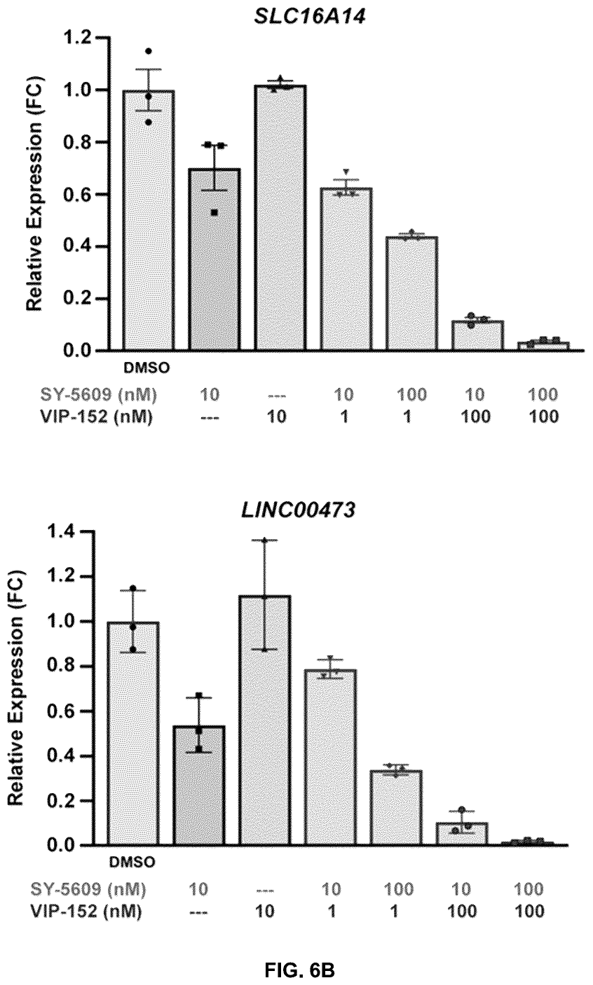

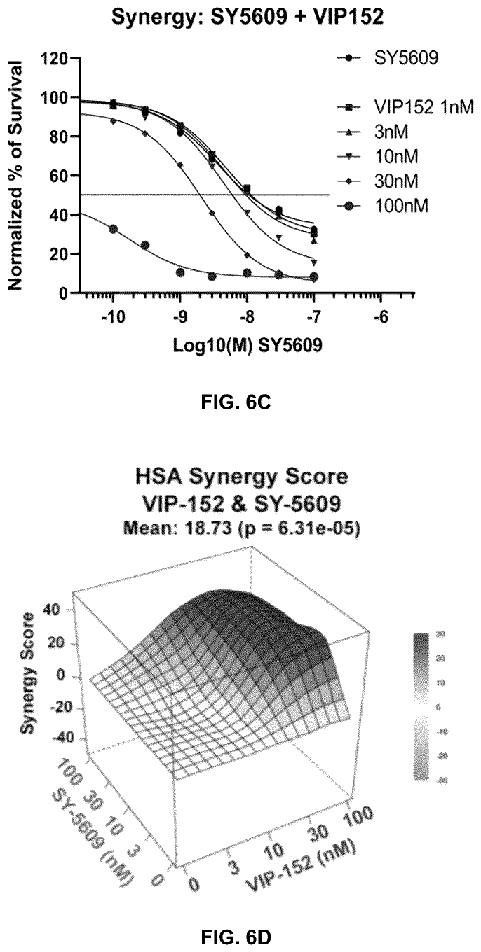

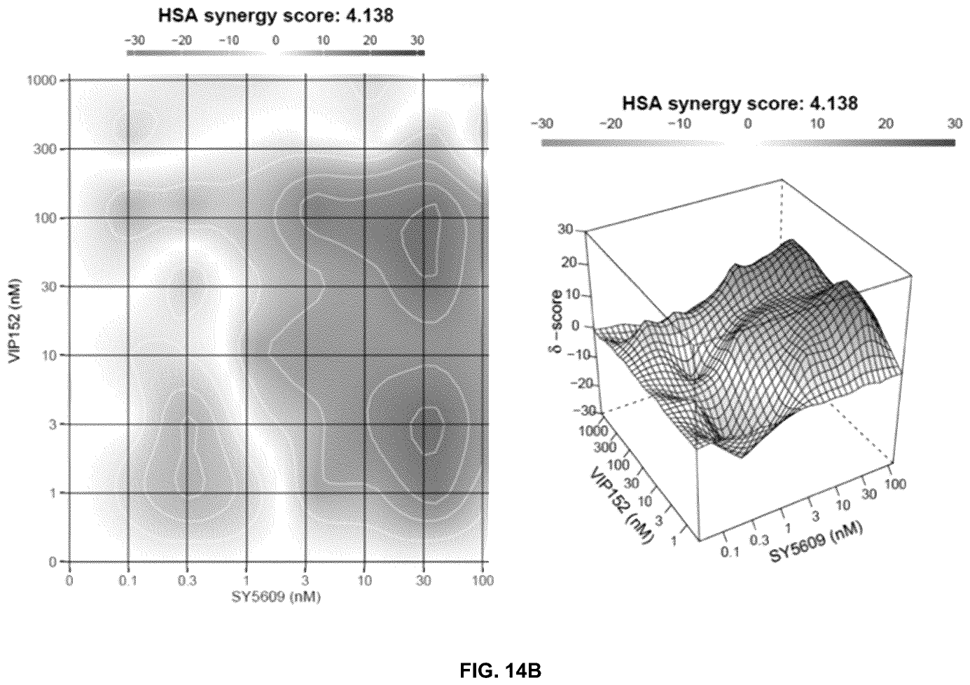

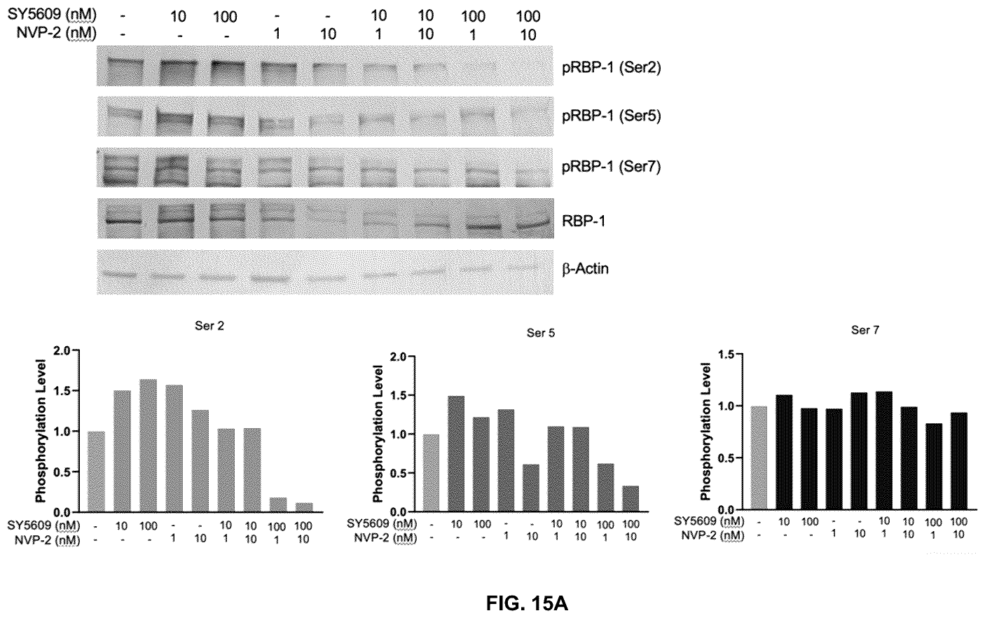

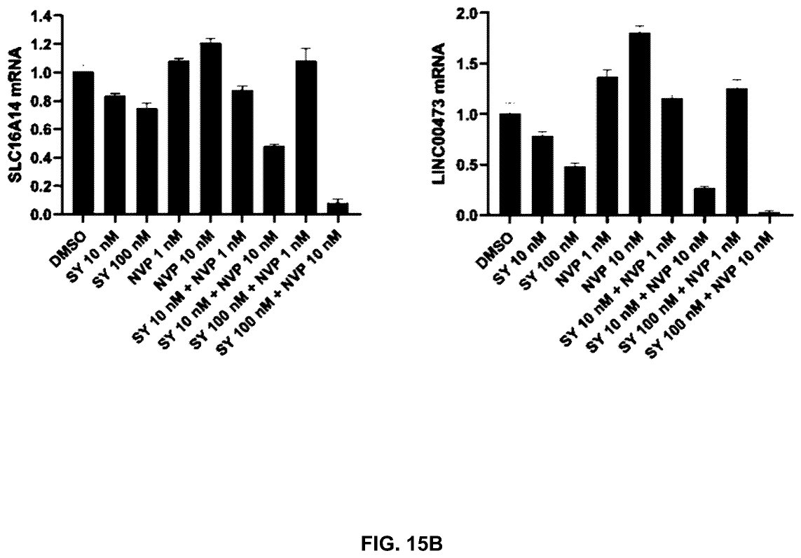

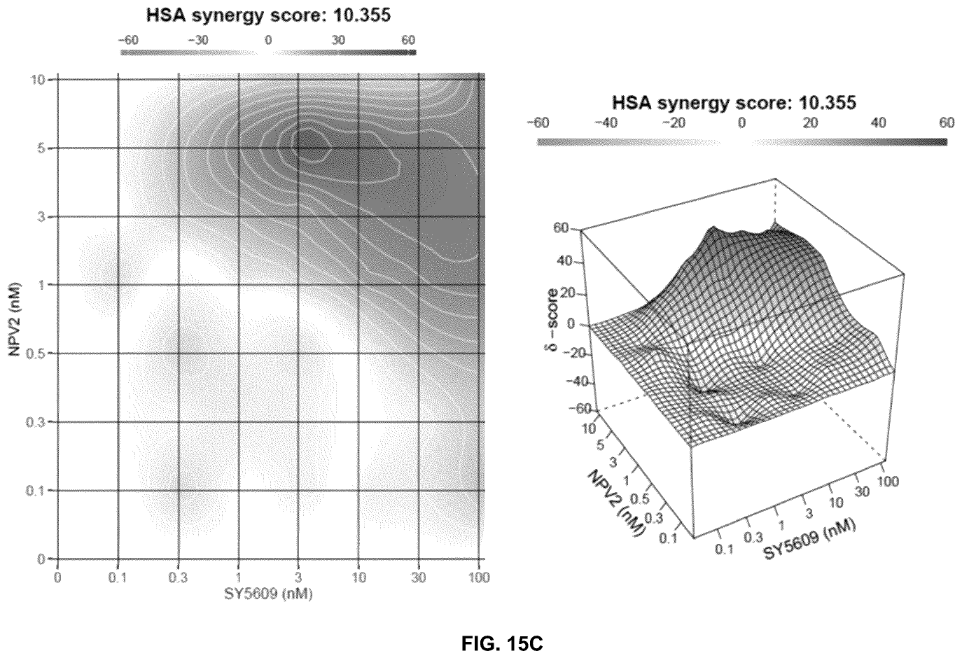

When treating DNAJB1-PRKACA expressing H33 cells with CDK7 inhibitors, a dose dependent reduction in SLC16A14 and LINC00473 expression was identified, with about 50% reduction in expression at 100 nM (FIGS. 3D-3E). Interestingly, treating the H33 cells with SY-5609 at 100 nM dose combined with VIP-152 (potent and specific CDK9 inhibitor) at the same dose resulted in markedly reduced RNA Pol II ser-2, -5 and -7 phosphorylation (FIG. 6A). Combination therapy also yielded abolished expression of FLC specific genes including SLC16A14 and LINC00473 (FIG. 6B). The combination demonstrated efficacy to a markedly greater degree than SY-5609 alone. Meanwhile VIP-152 alone resulted in no change in expression of the FLC-specific genes. To evaluate further, the combination of SY-5609 and VIP-152 on cell survival of DNAJB1-PRKACA expressing H33 cells was tested, and found robust synergistic effect at physiologically relevant doses, with overall synergy score of 18.73 (HSA metric) across the combined doses (FIGS. 6C-6D). For example, synergy scores at 30 nM VIP-152 and 30 nM SY-5609 were ˜38, showing remarkable synergistic lethality with only 10% of cells surviving. This experiment was repeated in primary human hepatocytes (PHH), which demonstrated an overall synergy score of 4.138 (HSA metric), this response was not nearly as robust at the H33 response (FIGS. 14A-14B). To confirm these results another selective CDK9 inhibitor, NVP-2, was tested. Treatment with SY-5609 in combination with NVP-2 resulted in markedly reduced RNA Pol II ser-2 and ser-5 phosphorylation FIG. 15A). Combination therapy also resulted in significantly decreased expression of the FLC specific genes SLC16A14 and LINC00473 (FIG. 15B). Finally, treatment of H33 cells with combination therapy of SY-5609 and NVP-2 demonstrated a significant synergistic effect with an overall synergy score of 10.355 (HSA score) across combined doses (FIG. 15C).

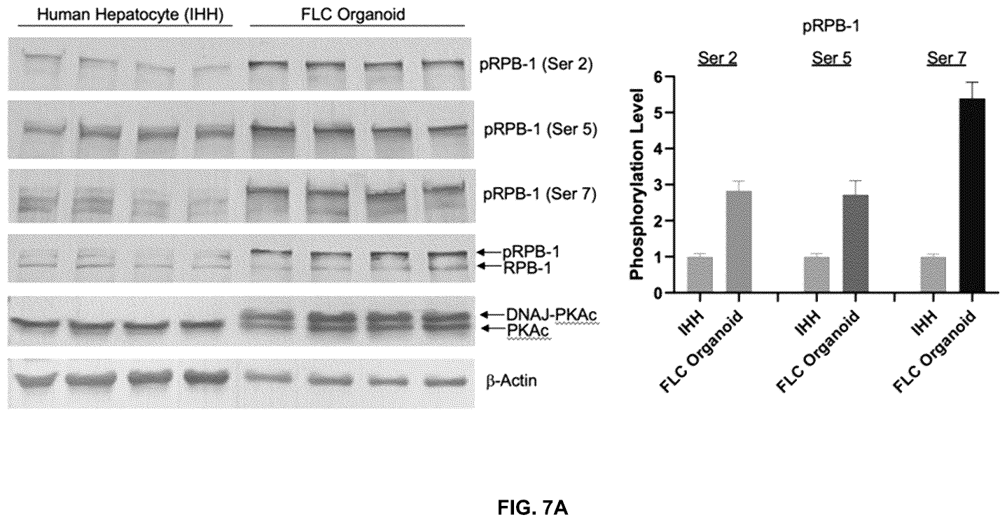

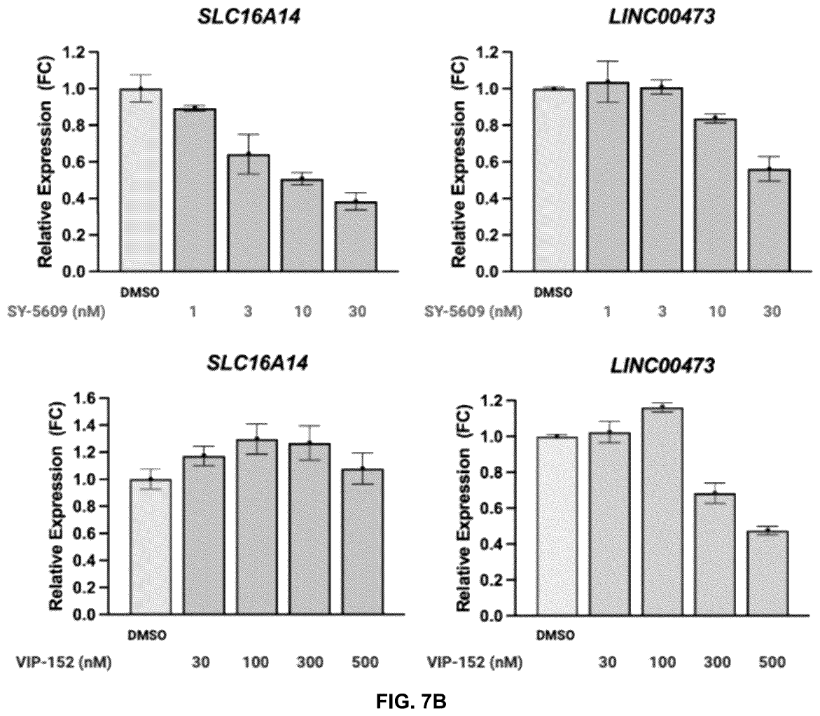

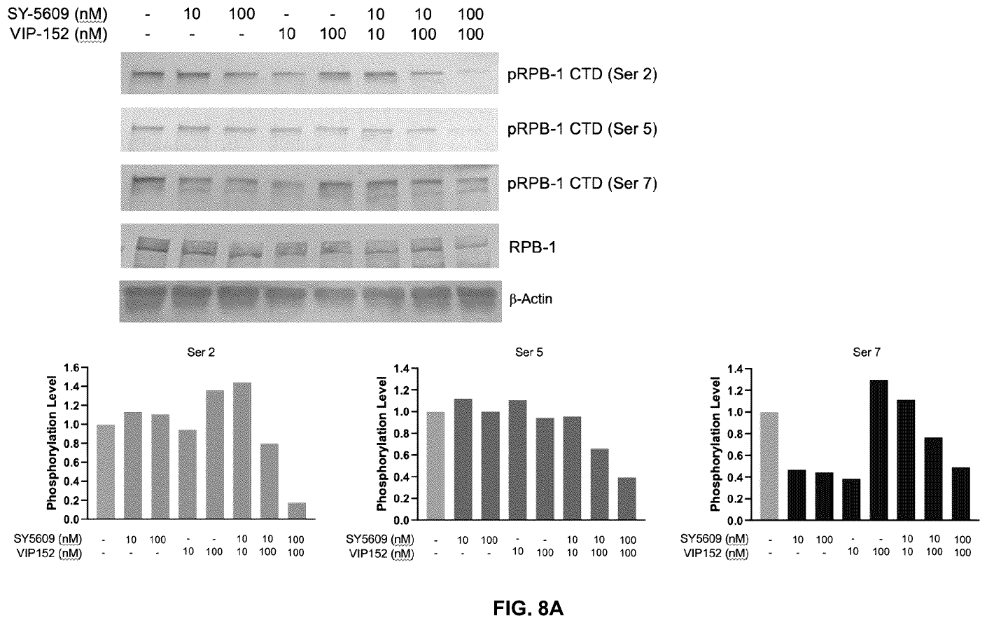

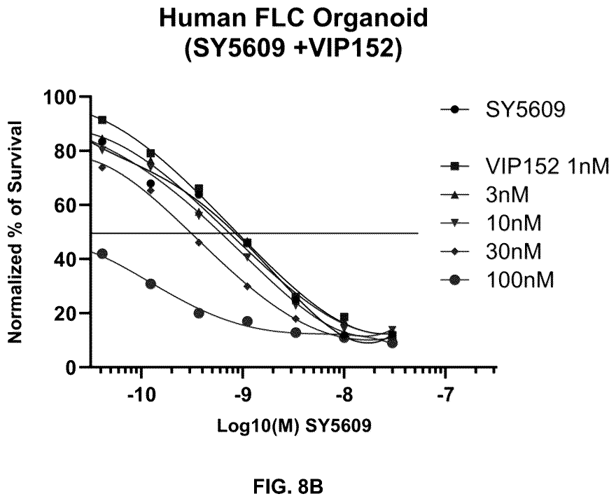

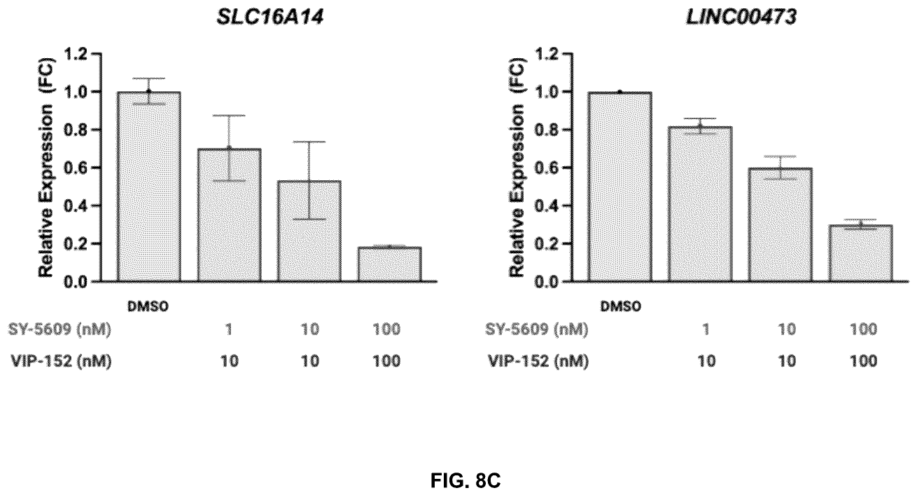

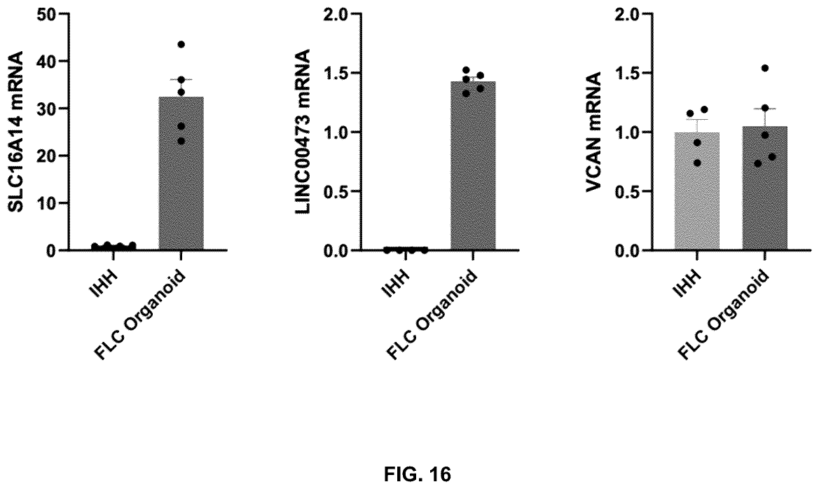

Following confirmation of the synergistic effect of treatment with SY-5609 and VIP-152 on both cell viability and expression of DNAJB1-PRKACA expressing cell targets SLC16A14 and LINC00473 in a 2D in vitro model, a patient cancer-derived organoid model from a patient with FLC was utilized. DNAJ-PKAc expression and increased RNA Pol II ser-2, -5 and -7phosphorylation as compared to immortalized human hepatocytes (IHH) was confirmed (FIG. 7A). The FLC specific genes SLC16A14 and LINC00473 were markedly increased in FLC organoid as compared to IHH, there was no significant difference in VCAN (FIG. 16). A dose dependent reduction in SLC16A14 and LINC00473 expression in response to treatment with escalating doses of SY-5609 and VIP-152 (FIG. 7B) was found. SY-5609 and VIP-152 combination therapy in these organoids was then tested, and a dose dependent decrease in RNA Pol II ser-2, -5 and -7 phosphorylation was found (FIG. 8A). Similar to the DNAJB1-PRKACA expressing H33 cells, a dose dependent decrease in cell viability with SY-5609 alone was identified, which was enhanced with concomitant VIP-152 treatment (FIG. 8B). The combination of SY-5609 and VIP-152 was then tested, and a greater reduction in SLC16A14 and LINC00473 expression in response to combination therapy compared to each single agent alone was found (FIG. 8C).

Thus, a novel druggable target in CDK7 has been uncovered that when inhibited causes FLC cell death and suppression of FLC specific gene expression. Moreover, the connection between DNAJB1-PRKACA, CDK7, RNA Pol II and super enhancer driven gene targets (SLC16A14 and LINC00473) in FLC is further strengthened by the synergistic combination therapy with CDK7 inhibition as the backbone (CDK7 inhibition+CDK9 inhibition). This combination will be evaluated in future work in vivo to optimize the tumoricidal effect while maintaining low toxicity.

### Discussion

The research landscape in FLC changed after a seminal study that revealed the presence of the DNAJB1-PRKACA gene fusion in human FLC, and was later shown to drive FLC in mouse (Oikawa et al. 2015, Kastenhuber et al. 2017, Dinh et al. 2020). The clinical importance of DNAJB1-PRKACA in human FLC was subsequently confirmed in two separate studies, including one in which the authors utilized shRNA spanning different regions of the DNAJB1-PRKACA breakpoint to demonstrate that the FLC cells were reliant upon DNAJB1-PRKACA for cell survival (oncogenic addiction) (Neumayer et al. 2023). This indicates that following tumor initiation, FLC requires the presence of DNAJ-PKAc (and downstream signaling) to survive. Separately, a group with expertise in vaccine therapy and immune targeting of cancer exploited the fact that the DNAJB1-PRKACA fusion transcript generates a protein product (DNAJ-PKAc) that is the clonal oncogenic driver in FLC, and consequently, FLC displays tumor-specific neoantigens that can be recognized by the immune system (Bauer et al. 2022). The authors found that unique fusion neoepitopes were predicted as ligands to the majority of HLA class II alleles and of HLA class I allotypes, and that CD8⁺ T-cells would recognize the neoepitopes, which was confirmed in vitro. They constructed a vaccine comprised of three separate HLA class I ligands (9-10 amino acids in length) and one longer peptide predicted to bind HLA class II (DP) to provide a personalized vaccine for a patient who had undergone multiple lines of surgery (including transplant) and systemic therapy. The vaccine was administered with a toll-like receptor ½ agonist, and the patient developed neoepitope-specific T-cells with objectively high intensity response to DNAJB1-PRKACA derived neoepitopes ex-vivo, as well as durable clinical response (no disease relapse at 21 months post-vaccination). Presumably, cells expressing DNAJ-PKAc were identified and targeted, and the absence of recurrence indicates that cancer cells could not ‘escape’ by selection for cancer cells without DNAJB1-PRKACA. Thus, combined, these two studies highlight the critical importance of DNAJB1-PRKACA in FLC, and that this fusion uniquely promotes a pathogenic mechanism within the cancer cells that is specifically reliant upon the presence of the DNAJ-PKAc protein. However, there has been inconclusive evidence of how DNAJ-PKAc drives cellular changes to promote neoplastic transformation and progression of FLC.

Therefore, identifying how the upstream driver of cellular changes, DNAJ-PKAc oncoprotein, modulates downstream targets to drive cell growth and metastasis should reveal a critical molecule or set of molecules that relay the aberrant signal. Evidence from a recent comprehensive analysis of human FLC revealed downstream reprogramming of enhancer regions to drive high levels of transcription of FLC-specific genes (Dinh et al. 2020), including specific downstream targets as a consequence of neoplastic change (e.g., SLC16A14 and LINC00473) (Honeyman et al. 2014, Dinh et al. 2020, Bauer et al. 2022, Neumayer et al. 2023). Evidence of abnormally increased CDK7 activation in human tumor vs normal tissue was identified, and the introduction of DNAJB1-PRKACA in human-derived cancer cells promoted aberrant activity of CDK7, suggesting a link to the DNAJ-PKAc oncoprotein. This was further substantiated by suppression of CDK7 causing inhibition of FLC-specific gene expression.

CDK7 is a serine/threonine protein kinase that exists as a CDK-activating kinase (CAK) and has gained significant interest as a target in cancer due to its dual roles in cell cycle regulation and transcriptional regulation (Yankulov and Bentley 1997, Fisher 2019). CDK7 targets the heptad sequence (Tyr-Ser-Pro-Thr-Ser-Pro-Ser (SEQ ID NO:10)) of the CTD in RNA Pol II, with preferential phosphorylation of serine-5 in the sequence (Larochelle et al. 2006). This is notable due to the fact that among the serine targets, serine-5 was identified as the most differentially phosphorylated in human FLC tumor versus normal liver and other liver cancer (HCC and CCA), and in H33 (HepG2^{DNAJB1-PRKACA}) versus HepG2 cells. Meanwhile, CDK substrates (e.g., CDK1, 2) are phosphorylated on the threonine residue (Thr-X-X-Val-Val-Thr-Leu (SEQ ID NO:11)), with Thr160 as the phospho-acceptor in CDK2. The location of CDK2 phosphorylation is interesting due to the preference of CDK7 to phosphorylate serine or threonine residues with an adjacent proline (+1 position) (Larochelle et al. 2006). Although the target substrates are structurally unrelated (e.g., RNA-Pol II CTD and CDK2), the ability of CDK7 to precisely and selectively recognize these substrate classes for phosphorylation was disentangled by Larochelle and colleagues (Larochelle et al. 2006). They found that CDK7 can exist as a dimer (with Cyclin H) or a trimeric complex (CDK7/Cyclin H/MAT1), and that only the MAT1 bound trimeric complex can phosphorylate RNA-Pol II CTD, the efficiency of which is increased by CDK7 phosphorylation. Meanwhile, both the dimeric and trimeric complex can phosphorylate the various CDK proteins (e.g., CDK1, CDK2), which was consistent with results from another study (Yankulov and Bentley 1997). Therefore, the distinct mechanisms of CDK7 function result in high specificity and selectivity for substrates, with CDK2 phosphorylation and RNA Pol II CTD phosphorylation representing the divergent outputs of CDK7 activation. This mechanistic insight not only accounts for the phosphoprotein results (i.e., preferential serine-5 of RNA Pol II CTD phosphorylation), but also explains the other phenotypic changes that occurred with CDK7 inhibition in the engineered cell line model (i.e., G0/G1 cell cycle arrest, suppression of FLC-specific super enhancer driven gene expression, potent FLC cell death in human models).

Selective CDK7 inhibitors were utilized to provide insight on the clinical relevance of targeting the aberrantly activated CDK7 pathway. For instance, SY-5609 was used throughout this study because it is a potent and selective CDK7 inhibitor with demonstrated effects on cell cycle, transcription and apoptosis in vitro and in vivo (Marineau et al. 2022). Notably, SY-5609 is currently in use in clinical trials, with a defined safety profile in humans (NCT04247126, NCT04929223) (Marineau et al. 2022). To confirm that the effects of SY-5609 were CDK7 dependent, a separate covalent binding CDK7 inhibitor (YKL-5-124) was utilized as well as siRNA to CDK7. The approach of using multiple methods was informed by a recent study where investigators who developed an older generation CDK7 inhibitor (THZ1) recognized that the effects initially attributed to CDK7 inhibition were instead due to CDK12/13 inhibition in addition to CDK7 inhibition (Kwiatkowski et al. 2014, Olson et al. 2019). These investigators then set out to develop a more specific inhibitor that maintained the irreversible binding to Cys312 of CDK7 (mechanism of THZ1). They generated YKL-5-124 by hybridizing the covalent warhead of THZ1 with the pyrrolidinopyrazole group from PF-3758309 (a PAK4 inhibitor) to substantially improve CDK7 selectivity (Olson et al. 2019). Meanwhile, the investigators that developed SY-5609 achieved high potency and selectivity for CDK7 without requiring a covalent warhead, and in an orally bioavailable form (Marineau et al. 2022). Through a series of structural modifications of a prior compound (SY-1365—derived from THZ1), they were able to generate SY-5609, which demonstrated a selectivity window of 4340-fold over the closest off-target (CDK16). In contrast to SY-1365, YKL-5-124 and THZ1, SY-5609 does not covalently bind to CDK7, yet maintains a high specificity through non-covalent interactions. Therefore, these two separate compounds as well as siRNA to CDK7 was used in initial studies (H33 cells) to support the involvement of aberrant CDK7 function in FLC and confirmed the efficacy of the selected compound (SY-5609) in patient-derived models of FLC.

In addition to promoting cell death in cells harboring the DNAJB1-PRKACA fusion, CDK7 inhibition was also shown to markedly suppress expression of SLC16A14 and LINC00473. These genes were identified from prior work that established unique super enhancer driven genes in human FLC (Dinh et al. 2020). Super enhancers within cancer cells are responsible for high levels of expression of genes that are essential for cell identity and survival (Hnisz et al. 2013, Chipumuro et al. 2014). These key genes associated with super enhancers control cell state and cancer hallmark qualities (Hnisz et al. 2013), highlighting the critical importance of prior work that identified genes associated with enhancer hotspots (Dinh et al. 2020). It was found that CDK7 may function as a regulator of the super enhancer-driven landscape in FLC, similar to prior preclinical data supporting CDK7 as a lead target for super-enhancer-driven cancer types (i.e., MYC) (Chipumuro et al. 2014, Christensen et al. 2014, Kwiatkowski et al. 2014). This idea is supported by a study by Neumayr et al. that shows the enhancer within the SLC16A14 gene activates an origin of replication core promoter dependent on CDK7 cofactors, strengthening the evidence that SLC16A14 expression in FLC is dependent on CDK7 activity (Neumayr et al. 2022). Notably, SLC16A14 is remarkably specific for FLC, with minimal expression across normal tissue types, other primary cancers and other metastatic cancers; this presents a highly intriguing and promising target. In addition, it was reasoned that FLC-specific therapeutic strategies should suppress these DNAJB1-PRKACA driven super enhancer associated genes, particularly SLC16A14 which serves as a strong marker of DNAJB1-PRKACA expressing cells. Future investigation will focus on elaborating the CDK7-driven effects including the transcriptional machinery (i.e., RNA Pol II) such that additional putative targets downstream of CDK7 can be identified for synergistic combination therapy.

Despite the compelling data from this current work, a key question that remains is how DNAJ-PKAc causes aberrant activation of CDK7 to promote transcriptional dysregulation. While the discovery advances the mechanistic understanding of DNAJ-PKAc in FLC, the proximity of CDK7 to DNAJ-PKAc is indeterminate. Specifically, it is unknown whether CDK7 is directly phosphorylated by the fusion oncoprotein, or whether intermediate proteins transmit the signal from DNAJ-PKAc. Although there is evidence that native PKA and CDK7 may have overlapping targets (e.g., nuclear retinoic acid receptor alpha) (Gaillard et al. 2006), currently, there is no evidence that native PKA directly regulates CDK7 activity. And while it is known that DNAJ-PKAc induces abnormal cAMP signaling and phosphorylates targets distinct from native PKA (Turnham et al. 2019, Zhang et al. 2020), the findings herein are the first evidence of altered CDK7 pathway activity due to the DNAJ-PKAc oncoprotein. Due to the complexity of CDK7 function (i.e., regulation of cell cycle and RNA Pol II is mediated by dimeric vs trimeric complex and phosphorylation status of CDK7) (Yankulov and Bentley 1997, Larochelle et al. 2006), there is likely a multifaceted interplay between DNAJ-PKAc and CDK7.

The data presented here are consequential and support a role of CDK7 in FLC. Targeting of CDK7 induces cell cycle arrest, apoptosis, and suppression of key FLC marker genes. Notably, this work also begins to untangle the regulation of super enhancer driven genes that are uniquely expressed in FLC as a result of DNAJB1-PRKACA (Dinh et al. 2020). Collectively, the basis for CDK7 inhibition as a therapy in human FLC is provided.

### Methods

### Human Tissue Samples

Normal liver, hepatocellular carcinoma (HCC), and cholangiocarcinoma (CCA) samples from patients were obtained from the Translational Science Biocore (TSB)-BioBank at the University of Wisconsin-Madison. FLC tissue samples (FLC 55, 57, 82, 83, 84, 87, 89, 106, 110, 109, 114) were obtained from the Fibrolamellar Cancer Foundation. Patient tumor derived fibrolamellar carcinoma (FLC4) for generation of the patient tumor derived organoid was obtained from the Fred Hutchinson Cancer Center.

### Cell Line and Culture

Hepatoblastoma HepG2 cell line was obtained from ATCC (Manassas, VA). Cells were maintained in RPMI1640 culture medium (Life Technologies, Carlsbad, CA) supplemented with 10% fetal bovine serum (Gemini Bio Products, Sacramento, CA) and 1% penicillin-streptomycin-glutamine (Life Technologies) at 37° C. and 5% CO₂. Immortalized Human Hepatocytes (IHHs) were obtained from Applied Biological Materials (T0063, Richmond, BC). IHHs were cultured in

William's E medium (Life Technologies, Carlsbad, CA) supplemented with 10% fetal bovine serum (Gemini Bio Products, Sacramento, CA) and 1% penicillin-streptomycin-glutamine (Life Technologies) on Collagen I (Rat tail)-coated culture dishes (Corning, New York, NY) at 37° C. and 5% CO₂.

### Generation of DNAJB1-PRKACA Expressing Cells

To generate HepG2^{DNAJB1-PRKACA} cells, mammalian dual-guide RNAs-CRISPR-CAS9-EGFP vector was designed (VectorBuilder, Chicago, IL). The guide RNA sequences were designed for excision of the region between the first introns of DNAJB1 and PRKACA on human Chromosome 19 using CRISPOR tool (gRNA1 (DNAJB1) 5′-CAGGAGCCGACCCCGTTCGT-3′ (SEQ ID NO:1), gRNA2 (PRKACA): 5′-GTAGACGCGGTTGCGCTAAG-3′ (SEQ ID NO:2)) (Concordet and Haeussler 2018). HepG2 cells were transfected with 10 μg of dual-guide RNAs-CRISPR-CAS9-EGFP vector and Lipofectamine 2000 transfection reagent (Invitrogen, Carlsbad, CA) and incubated at 37° C. and 5% CO₂ for 2 days. Single GFP positive cells were sorted at 4° C. using a BD FACSAria III (BD Biosciences, Franklin Lakes, NJ) and deposited in 1% gelatin-coated 96 well plates. The excision of the DNAJB1-PRKACA region was confirmed by PCR. The PCR was performed by using a forward primer (DNAJB1-intron 1-F, 5′-AGCTTCTAGCATGTCTGGGG-3′ (SEQ ID NO:3)), and a reverse primer (PRKACA-intron 1-R, 5′-CTGGGAAGGCTCATGAGACCT-3′ (SEQ ID NO:4)) for DNAJB1-PRKACA fusion site on the genome. Genomic DNA was extracted from clones using Wizard Genomic DNA purification kit (Promega, Madison, WI) and PCR was conducted at a temperature of 98° C. for 5 minutes (1 cycle), 98° C. for 30 seconds, 58° C. for 45 seconds and 72° C. for 1 minute (32 cycles) and 72° C. for 10 minutes (1 cycle) using GoTaq G2 DNA polymerase (Promega). Additionally, DNAJB1-PRKACA fusion mRNA expression was confirmed by reverse-transcription (RT)-PCR. Total RNA was extracted from cells using the Qiagen RNeasy kit (Qiagen, Valencia CA). Complementary DNA was synthesized using High-Capacity cDNA Reverse Transcription Kit (Applied Biosystems, Foster city, CA) as per the manufacturer's instruction and PCR was carried out at a temperature of 95° C. for 5 minutes (1 cycle), 95° C. for 30 seconds, 56° C. for 30 seconds and 72° C. for 30 seconds (30 cycles) and 72° C. for 5 minutes using GoTaq DNA polymerase (Promega) and a forward primer (hChimera exon 1-F, 5′-GTTCAAGGAGATCGCTGAGG-3′ (SEQ ID NO: 5)), a reverse primer (hChimera exon 3, 5′-TTCCCGGTCTCCTTGTGTTT-3′ (SEQ ID NO: 6)). The amplified PCR products were isolated and subcloned into pGEM-T easy vector (Promega) and sequenced using Bigdye terminator v3.1 (Applied Biosystems). The Sequence was analyzed by SnapGene software (San Diego, CA).

### Cell Viability Assay

To determine the changes in cell proliferation in HepG2 cells and HepG2^{DNAJB1-PRKACA} clones, CellTiter Glo 2 assay systems (Promega) were used. Approximately 2.0×10³ cells/well were seeded in a 96-well plate and incubated in RPMI1640 culture medium over 4 days at 37° C. and 5% CO₂. After addition of the CellTiter Glo 2 reagent to cell culture medium in 96 well plate and incubation for 10 minutes, luminescence was measured by CLARIOstar Plus microplate reader (BMG Labtech, Ortenberg, Germany). Additionally, the growth of HepG2 cells and HepG2^{DNAJB1-PRKACA} clones were measured using hemocytometer cell counting. Cells (5.0×10⁴ cells/well) were seeded in a 6-well plate and incubated in RPMI1640 culture medium for 4 days at 37° C. and 5% CO₂. Cells were trypsinized, stained with 0.4% Trypan Blue (Invitrogen) and counted using hemocytometer (Day 0, 2 and 4).

For drug-response analysis, HepG2 cells and HepG2^{DNAJB1-PRKACA} clones (H12, H33) (2.0×10³ cells/well) were seeded in a 96-well plate and incubated with RPMI1640 cell culture medium for 24 hours. After incubation, cells were treated with SY-5609 (1 pM-10 μM) (MedChemExpress, Monmouth Junction, NJ), YKL-5-124 (100 pM-10 μM) (MedChemExpress) or vehicle (DMSO) for 48, 72 hours or 120 hours. Cell viability was measured using CellTiter Glo 2 reagent. Percent of survived cells and growth inhibition were calculated relative to vehicle control (as 100% of survival). The dose-response curves and IC₅₀ values were analyzed using GraphPad Prism software (San Diego, CA). Differences between conditions were tested with a t-test and a p<0.05 was taken as statistically significant.

### Caspase 3/7 and 8 Assay

To measure the apoptosis activity in HepG2 cells and HepG2^{DNAJB1-PRKACA} clones, Caspase-Glo 3/7 and 8 assay systems (Promega) were used. Approximately 2.0×10³ cells/well were seeded in a 96-well plate and (1) incubated in RPMI1640 culture medium over 4 days at 37° C. and 5% CO₂ or (2) incubated in RPMI1640 culture medium containing SY-5609 (0.1-10 μM) (MedChemExpress) or DMSO for 24 hours. After addition of the Caspase-Glo 3/7 or 8 reagent to cell culture medium in 96 well plate and incubation for 30 minutes, luminescence was measured by CLARIOstar Plus microplate reader (BMG Labtech).

### Cell Cycle Analysis

To synchronize cell cycle in HepG2 cells and HepG2^{DNAJB1-PRKACA} clones, cells were treated with serum starvation medium (RPMI1640 medium (Life Technologies) supplemented with 1% penicillin-streptomycin-glutamine (Life Technologies)) at 37° C. and 5% CO₂ for 16 hours. Then serum starvation medium was (1) replaced to RPMI1640 culture medium and cells were collected over 24 hours (every 6 hours) or (2) replaced to RPMI1640 culture medium containing SY-5609 (1 μM) (MedChemExpress) or DMSO for 24 hours and cells were collected. The collected cells were fixed with 70% ethanol and stained with 50 μg/mL propidium iodide (BioLegend, San Diego, CA) and 100 μg/mL RNAse A (Thermo Fisher). Samples were sorted with an Attune NxT flow cytometer (Thermo Fisher) and the DNA content was analyzed for the percent of cells in G0/G1, S, and G2/M phase with ModFit LT 6.0 software at the University of Wisconsin Carbone Cancer Center Flow Cytometry Laboratory. The average coefficient of variation (CV) of each sample was <6%. Differences between conditions were tested with a t-test and a p<0.05 was taken as statistically significant.

### RNA Isolation, Sequencing, Differential Gene Expression Analysis

RNA isolation and RNA sequencing was performed as previously described (Schwartz et al. 2021). Briefly, to evaluate for transcriptomic differences between HepG2 and HepG2^{DNAJB1-PRKACA} clones (H33 and H12 cells), bulk RNA-seq was performed on 6 biological replicates collected from each cell line. RNA Isolation was carried out using the RNeasy protocol (Giagen, Hilden, Germany) according to the manufacturer's recommendations. Quality was tested for an RNA integrity number (RIN)>7.5 on the Agilent 2100 bioanalyzer (Agilent Technologies, Santa Clara, CA). A total of 300 ng of mRNA was enriched with poly-A selection and sequencing on the Illumina HiSeq2500 platform by the University of Wisconsin Biotechnology Sequencing Core. FASTq files were processed with Skewer and genes were filtered to remove those with low expression (Jiang et al. 2014). Samples were normalized by the method of trimmed mean of M-values (Robinson and Oshlack 2010). Contrasts were drawn with the edgeR package, with differential expression taken when the FDR q<0.05 (Robinson et al. 2010). Pathway testing was performed with the KEGG database (Kyoto Encyclopedia of Genes and Genomes) using previously described methods (Wu et al. 2021). The top 500 significant genes were inputted, ordered by q value, and the top significant pathways ordered by q-value (an adjusted multiple testing p-value found using the false discovery rate approach of Benjamini and Hochberg) (Benjamini et al. 2001) were plotted for visualization. Pathway dot size is indicative of the number of genes in each pathway. All RNA seq data is available at Gene Expression Omnibus (GEO) accession number GSE232374.

### Quantitative Reverse Transcription PCR (RT-qPCR)

HepG2 and HepG2^{DNAJB1-PRKACA} clones were incubated with RPMI1640 cell culture for 24 hours, treated with SY-5609 (1-300 nM) (MedChemExpress), YKL-5-124 (1-300 nM) (MedChemExpress) or vehicle (DMSO) for 24 hours. Total RNA was isolated from cells using the Qiagen RNeasy kit (Qiagen, Valencia CA). The isolated RNAs (20 ng) were reverse-transcribed and quantitative PCR (qPCR) were perform using GoTaq Probe 1-Step RT-PCR system (Promega) in a QuantStudio 7 Flex Real-Time PCR System (Applied Biosystems). The reverse-transcription was conducted at a temperature of 45° C. for 15 minutes, and 95° C. for 2 minutes during one cycle. The denaturation, annealing and extension were performed at 95° C. for 15 seconds and 60° C. for 1 minutes (45 cycles). The mRNA levels were measured with Taqman Gene Expression System (FAM-MGB) (Applied Biosystems) (Assay ID: DNAJB1: Hs00356730_g1, DNAJB1-PRKACA fusion: Hs05061318_ft, SLC16A14: Hs00541300_m1, LINC00473: Hs00293257_m1, VCAN: Hs00171642_m1, OAT: Hs00236852_m1, GAPDH: Hs02786624_g1, B-Actin: Hs01060665_g1). To detect PRKACA mRNA expression, the qPCR primers (Primer 1: 5′-AGCGGGACTTTCCCATTT-3′ (SEQ ID NO:7), Primer 2: 5′-AAGAAGGGCAGCGAGCA-3′ (SEQ ID NO:8)) and FAM-labeled probe (5′-FAM-TGGCTTTGGCTAAGAATTCTTTCACGC-3′ (SEQ ID NO:9)) were designed using PrimerQuest Tool (IDT). The qPCR primers and probe spanned the boundary of exon 1-2 junction since DNAJB1-PRKACA fusion gene harbors exon 2-10 of PRKACA gene. GAPDH and β-Actin mRNA levels were used as internal control and the data were analyzed using the ^{ΔΔ}Ct method.

### Immunoblotting

HepG2 cells and HepG2^{DNAJB1-PRKACA} clones were lysed and homogenated in CelLytic M lysis reagent (Sigma-Aldrich) with Halt protease and phosphatase inhibitor cocktail (Thermo Fisher). IHHs, FLC tumors and FLC organoids were lysed and homogenated in RIPA Lysis buffer (Thermo Fisher) with Halt protease and phosphatase inhibitor cocktail (Thermo Fisher). For immunoblotting, 20 μg of whole cell extract or 40-80 μg of liver homogenate was loaded on Mini-Protean TGX protein gel (4-20% or 7.5%) (Bio-Rad, Hercules, CA) and transferred to Immobilon-P membranes by electrophoresis (MilliporeSigma, Burlington, MA). Membranes were incubated in 10% w/v BSA blocking buffer (Thermo Fisher) at room temperature for 1 hour and hybridized with primary antibody (PKA(c): 610981 (BD Biosciences), CDK2: #2546 (Cell Signaling Technology), phospho-CDK2 (Thr160): #2561 (Cell Signaling Technology), CDK7: #2916 (Cell Signaling Technology), phospho-CDK7 (T170): ab155976 (abcam, Cambrige, MA), RNA polymerase II RPB-1: RPB-1 NTD; #14958, Phospho-CTD (Ser2); #13499, Phospho-CTD (Ser5); #13523, Phospho-CTD (Ser7); #13780 (Cell Signaling Technology), SCG2; PA5-115018 (Thermo Fisher), PARP; #9542 (Cell Signaling Technology), Cleaved PARP (Asp214); #5625 (Cell Signaling Technology), β-Actin: #4967 (Cell Signaling Technology), MAB8929 (R&D Systems, Minneapolis, MN) at 4° C. overnight. After hybridization with Alkaline Phosphatase (AP)-conjugated secondary antibody (anti-mouse: #7056 (Cell Signaling Technology), anti-rabbit: 111-055-144 (Jackson ImmunoResearch, West Grove, PA)), proteins were detected using NBT/BCIP solution (Thermo Fisher). The samples were loaded separately and membranes were developed independently. The protein levels were quantified using ImageJ software (Fiji).

siRNA Transfection

HepG2 cells and HepG2^{DNAJB1-PRKACA} clones were transfected in 96-well plate with 0.25-100 nM Silencer small interfering RNA (siRNA) (Negative Control: #1, CDK7: s2830) (Life Technologies) and Stealth RNAi (CDK7: CDK7VHS40192) (Invitrogen) using Lipofectamine RNAiMAX transfection reagent (Life Technologies). Four days after the transfection, cell viability was measured using CellTiter-Glo 2 reagent (Promega). After addition of the reagent to cell culture medium, luminescence was measured by CLARIOstar Plus microplate reader (BMG Labtech). For immunoblotting, HepG2^{DNAJB1-PRKACA} cells (H33 clone) were transfected with siRNA (negative control or siCDK7, 1-10 nM) using Lipofectamine RNAiMAX transfection reagent for 48 hours. Whole cell extracts were prepared using CelLytic M lysis reagent (Sigma-Aldrich) with Halt protease and phosphatase inhibitor cocktail (Thermo Fisher).

### Primary Human Hepatocyte

Primary human hepatocytes (PHHs) were prepared from fresh human liver samples. Liver samples were perfused using perfusion solution I and liver tissues were dissociated by Digestion-Solution containing collagenase P (Kegel et al. 2016). PHHs were isolated and purified from hepatocyte rich cell suspension using 25% Percoll solution (Sigma-Aldrich) for density gradient centrifugation. Approximately 2.0×10⁴ purified PHH cells/well were seeded in rat tail collagen (collagen type I) (Life Technologies) coated 96-well plate and incubated in hepatocyte Incubation medium at 37° C. and 5% CO₂ (Kegel et al. 2016). Twenty-four hours after incubation, PHHs were treated with SY-5609 (100 pM-10 μM) (MedChemExpress) or vehicle (DMSO) for 2-5 days. Cell viability was detected using CellTiter Glo 2 reagent (Promega). After addition of the reagent to cell culture medium, luminescence was measured by CLARIOstar Plus microplate reader (BMG Labtech).

### Drug Combination Study

To investigate the synergistic effect between SY-5609 (CDK7 inhibitor) and VIP-152 or NVP-2 (CDK9 inhibitor), HepG2^{DNAJB1-PRKACA} (H33) cells (2.0×10³ cells/well) were seeded in an 8×8 matrix in a 96-well plate and incubated with RPMI1640 cell culture medium for 24 hours at 37° C. and 5% CO₂. Cells were treated with SY-5609 (0.1-100 nM), VIP-152 (1-100 nM), NVP-2 (0.1-10 nM) or vehicle (DMSO) for 72 hours. Cell viability was measured using CellTiter Glo 2 reagent. Percent of survived cells and growth inhibition were calculated relative to vehicle control (as 100% of survival). Synergy scores and the dose-response curves were analyzed using SynergyFinder tool. Drug synergy was evaluated using the highest single-agent (HSA) model and Loewe model (Ianevski et al. 2017). The combination effect of SY-5609 and VIP152 on FLC-specific gene expression and CDK7-mediated RNA Pol II activity was examined in HepG2^{DNAJB1-PRKAC} (H33) cells. HepG2^{DNAJB1-PRKAC} (H33) cells were treated with SY-5609 (10-100 nM) and VIP-152 (1-100 nM), NVP-2 (1-10 nM) or vehicle (DMSO) for 24 hours. The total RNA and whole cell lysate were isolated for RT-qPCR and immunoblotting (described above).

### FLC Human Cell Line

FLC-H (FLX1) cells were generated from a patient-derived xenograft model (Oikawa et al. 2015) and grown in RPMI1640 media containing 300 mg/L 1-glutamine (Thermo Fisher Scientific) supplemented with 10% fetal bovine serum (Thermo Fisher Scientific), 1% penicillin-streptomycin (Thermo Fisher Scientific) and 2.5 μg/mL human hepatic growth factor (Thermo Fisher Scientific). Cells were cultured in a humid chamber at 37° C. and 5% CO₂. FLC1025 and FLCmet3 cells were generated directly from a human FLC tumors (Fred Hutchinson Cancer Center) and maintained in DMEM/F12 supplemented with 10% FBS (Thermo Fisher Scientific), 0.04 μg/mL dexamethasone (Thermo Fisher Scientific), 0.1% gentamicin (Thermo Fisher Scientific), 1 μg/mL recombinant human insulin (Thermo Fisher Scientific), 0.55 μg/mL human transferrin (Thermo Fisher Scientific), and 0.5 ng/mL sodium selenite (Thermo Fisher Scientific). The media was supplemented with 10% DMEM from cultured human embryonic kidney cells harboring a human RSPO1 transgene and 20 μM Y-27632 ROCK inhibitor. FLC cells were plated at a density of 0.1×10⁶ cells/well in 12-well plates. After overnight incubation, cells were treated with vehicle control or SY-5609. Six days post-treatment cells were harvested for counting by hemocytometer. Cell viability was determined using trypan blue staining. Cell counting was performed in three biological replicates per trial and two trials were performed for each condition.

### FLC Human Organoid

Patient tumor derived fibrolamellar carcinoma (FLC4-PDX) for generation of the patient tumor derived organoid was obtained from the Fred Hutchinson Cancer Center (Gritti et al. 2024). Xenograft tumors were then developed from the FLC4-PDX tissue in Rag2/IL2RG (R2G2) mice at the University of Wisconsin-Madison. 3D organoid models were developed from the xenograft tumors. Organoids were grown and maintained in 50% growth factor reduced Cultrex matrix (R&D Systems) overlayed with Advanced DMEM/F12 (Gibco) media supplemented with 1× Glutamax (Gibco), 1% penicillin-streptomycin (VWR), 10 mM HEPES (Gibco), 10 mM nicotinamide (Sigma), 0.5 mM N-acetyl-L-cysteine (Sigma), 0.4× B27 without vitamin A (Gibco), 500 nM A83-01 (Sigma), 500 nM SB202190 (MedChem Express), 500 pM Wnt surrogate fusion peptide (ImmunoPercise), 100 ng/mL human recombinant noggin (Acro), 50 ng/ml human recombinant EGF (Gibco), 500 pM HA tagged R-Spondin 1 (produced in house), and 10 nM Y27632 (MedChem Express). Dose response curves were produced by plating FLCPDX organoids at a density of 5000 cells per well in a 96 well plate (Ibidi). After 24 hours, cells were treated with SY-5609, VIP-152, combination, or DMSO control. VIP-152 was removed after 24 hours of treatment. All media was replaced after 24 hours and 72 hours. Viability was determined using 3D CellTiterGlo (Promega) at 144 hours. Human FLC organoid were plated for qPCR samples in 12 well plates (Thermo Fisher) and allowed to reach confluency. Confluent plates were treated with SY-5609, VIP-152, combination, or DMSO control. VIP-152 was removed and all media across the experiment was changed at 24 hours. After 96 hours, organoids were removed from the plates and washed twice with 1× PBS. Pellets were resuspended in 250 μL of RNAlater (Sigma) and frozen at −80 C until RNA extraction.

### Tissue Slice Preparation

FLC tumor slices were prepared as described previously (Sivakumar et al. 2019, Nishida-Aoki et al. 2020). Briefly, dissected tumor tissues were cut into 400-μm organotypic tumor slices using the Leica VT1200S vibratome microtome (Nusslock) with HBSS as the cutting medium. The slices were then cut into 400-μm cuboids using a McIlwain tissue chopper (Ted Pella) as described previously (Sivakumar et al. 2019). Cuboids were immediately placed into 96-well ultralow-attachment plates (Corning) and incubated with Williams' medium containing 12 mM nicotinamide, 150 nM ascorbic acid, 2.25 mg/mL sodium bicarbonate, 20 mM HEPES, 50 mg/mL additional glucose, 1 mM sodium pyruvate, 2 mM L-glutamine, 1% (v/v) ITS, 20 ng/ml EGF, 40 IU/mL penicillin, and 40 μg/mL streptomycin containing RealTime Glo reagent (Promega) according to the manufacturer's instructions. After 24 h, the baseline cell viability of cuboids was measured by RealTime Glo bioluminescence using the Synergy H4 instrument (Biotek). Cuboids were exposed to DMSO (control), SY-5609 (0.1-1 μM) or Staurosporine (0.5 μM; positive control), and overall tumor tissue viability was measured daily, up to 7 d after treatment.

### Statistical Analysis

All analyses were performed with GraphPad Prism software (San Diego, CA) unless otherwise indicated. Samples were not excluded from the analysis unless explicitly stated. Standard comparisons of the mean were performed with a t-test with standard deviation unless explicitly stated.

### Example 2. Identify Efficacy of CDK7Inhibition In Vivo in Patient-Derived Xenograft Models of FLC

### Background

Strong evidence has been generated in several preclinical models that CDK7 serves as a therapeutic target in FLC. A better understanding of this target in FLC will stem from experiments showing CDK7 inhibition in patient-derived xenografts (PDXs): (i) suppresses phosphorylation of CDK7 target proteins in the tumor, (ii) suppresses FLC-specific super enhancer driven genes, (iii) produces minimal toxic side effects, and (iv) causes tumor cell death. This will further substantiate the clinical use of SY-5609 in treatment of FLC.

### Experiments

Determine Dose of SY-5609 that Suppresses the CDK7 Pathway in Tumor Cells In Vivo

Following orthotopic injection of patient tumor into R2G2 mice and established tumor formation, the range of 2 mg/kg daily to 6 mg/kg daily will be assessed and evaluated for CDK7 inhibition in the tumor after 1 week of treatment (Marineau et al. 2022, Henry et al. 2021). Protein will be isolated from the tumors of control treated (n=4) and SY-5609 treated (n=4) mice for each of the doses (2 mg/kg, 3 mg/kg, 4 mg/kg, 5 mg/kg and 6 mg/kg). The molecular endpoints of p-CDK2, CDK2, p-Ser-5 RNA Pol II and total RNA Pol II levels will be compared to control treated tumors to ensure achieve CDK7 inhibition in the tumor tissue is achieved. Furthermore, part of the tumor will be preserved for subsequent analysis (see below).

### Confirm Safety Profile of CDK7 Inhibition

At the identified dose(s) that demonstrate efficacy in suppressing the CDK7 pathway, the tolerance of SY-5609 treatment to C57BL/6J and R2G2 mice that do not harbor tumors will be tested. Previous work has shown that SY-5609 is well tolerated in vivo (Henry et al. 2021). Age 8-10 week mice will be treated with SY-5609 daily versus vehicle control (n=10 per group for each genotype and each dose) and mice monitored out to treatment of seven weeks (Marineau et al. 2022). Bodyweight will be measured every 3 days. Further, at necropsy, stomach, small intestine, colon, kidney, pancreas, liver, and lung (n=3 per group for each genotype) will be harvested, and histologic assessment of each organ will be performed to identify any subclinical evidence of organ dysfunction (e.g., inflammation).

Confirm Suppression of FLC-Specific Super Enhancer Driven Genes with CDK7 Inhibition

At the doses that demonstrate efficacy in suppressing the CDK7 pathway (see above), changes in FLC-specific super enhancers and super enhancer driven gene expression will be assessed. RNA isolation and RNA sequencing analysis will be performed in SY-5609 treated (n=6) versus control treated tumors (n=6), including differential gene expression analysis, principal component analysis, hierarchical clustering and pathway analysis as previously described (Nukaya et al. 2023, Schwartz et al. 2023). Furthermore, suppression of FLC-specific genes (e.g., SLC16A14, LINC00473) (Dinh et al. 2020) will be assessed. Finally, ChRO-seq and analysis (PS lab) will be performed to evaluate for differences in FLC specific transcriptional regulatory elements that occur with CDK7 inhibition (Dinh et al. 2020).

### Determine Therapeutic Efficacy of CDK7 Inhibition In Vivo

R2G2 mice harboring orthotopically implanted tumors will undergo treatment with SY-5609 (n=6) versus control (n=6) for seven weeks at which point mice will be sacrificed and tumors harvested. Tumor size will be measured (i.e., differences in tumor growth); part of the tumor will be assessed for differences in apoptosis as measured by cleaved PARP (Nukaya et al. 2014); and histologic sections of tumors will be generated to evaluate for differences in mitotic figures and Ki67 index. This will provide insight on the efficacy of single-agent CDK7 inhibition in patient-derived models of FLC.

### Results

Experiments were conducted in accordance with the methods outlined above. To evaluate the safety and efficacy of CDK7 inhibition in mice bearing patient-derived DNAJB1-PRKACA driven cancer, ten immunodeficient mice (R2G2) were injected in the left flank with 1×10⁵ FLC PDX cells at 8-10 weeks of age. These cells were obtained from a patient with FLC and harbor the DNAJB1-PRKACA gene fusion, causing expression of DNAJ-PKAc fusion oncoprotein (FIG. 17A). Tumor growth occurred until palpable tumors formed, and baseline measurements were obtained. Mice labeled FLC1-5 were administered corn oil vehicle control via oral gavage on days 1-14. Mice labeled FLC6-10 were administered the CDK7 inhibitor SY-5609 at a dose of 5 mg/kg in corn oil via oral gavage on days 1-14. Tumor size was measured at Day 0, Day 7 and Day 14 of therapy, at which point mice were sacrificed, tumors removed, final tumor volumes calculated, and tumor preserved for molecular and histologic analysis. Remarkably, treatment with a CDK7 inhibitor (SY-5609) resulted in suppression of phosphorylated CDK7 (FIG. 17A) and dramatic decrease in size of tumors compared to corn oil control (*p=0.008 on Day 7, **p =0.001 on Day 14 for corn oil versus SY-5609) (FIGS. 17B-17D). Furthermore, the mice tolerated treatment well with mice maintaining weight and good health throughout the study (FIG. 17E).

Also shown is monotherapy with the CDK9 inhibitor VIP-152, which showed tumor growth suppression compared to control treated mice (FIG. 17F). YKL-5-124 and other CDK7 inhibitors such as samuraciclib will also be tested and are predicted to have the same therapeutic effects.

### Example 3. In Vivo Synergy of Combination Therapies

### Background

To begin to understand how the DNAJ-PKAC signaling cascade induces epigenetic remodeling, the DNAJB1-PRKACA gene fusion was introduced at the native promoter of HepG2 cells (Nukaya et al. 2023). Using this model system, a potential mediator of DNAJ-PKAc driven pathogenesis was identified: aberrant CDK7 activation. Abnormally increased CDK7 activation in human tumor vs normal tissue and human-derived cancer cells (Nukaya et al. 2023) was confirmed. Interestingly, CDK7 has a known role in cancer associated super enhancers due to its function in phosphorylating RNA Polymerase II (RNA Pol II) (Chipumuro et al. 2014, Larochelle et al. 2012, Kwiatkowski et al. 2014, Christensen et al. 2014). RNA Pol II, along with other cofactors and chromatin regulators, is highly enriched at super enhancers and can transcribe enhancer RNA that contributes to enhancer function (Hnisz et al. 2013). Concordantly, through informed and targeted studies, synergistic partners that enhance the tumor cell killing effect of CDK7 inhibitors have been identified in preclinical models. The synergy of these combination therapies will be shown in FLC patient-derived xenografts and patient-derived organoid models.

### Experiments

### Determine Efficacy of Combination Therapy Strategies Based on CDK7 Inhibition

The synergy in vivo of SY-5609+VIP152 and YKL-5-124+VIP152 will be shown. Prior work in preclinical models that informed clinical trials (other cancers) has shown efficacy in the range of 6 mg/kg-15 mg/kg qweekly for VIP152 (Sher et al. 2023). Similar to CDK7 inhibition, the drug dose at which VIP152 induces suppression of p-Serine 2 (RNA Pol II) in tumors will be established, and toxicity at the various doses will also be assessed. Combination treatment of SY-5609+VIP152 will then be performed at the lowest effective dose of VIP152 given the anticipated synergistic effect. Furthermore, NVP2 will also be combined with CDK7 inhibition (SY-5609/NVP2 and YKL-5-124/NVP2). Mouse weight will be evaluated every three days to assess for toxicity. At the end of seven weeks, mice will be sacrificed and tumors harvested. Tumor size will be measured (i.e., differences in tumor growth); part of the tumor will be assessed for differences in apoptosis as measured by cleaved PARP (Nukaya et al. 2023); and histologic sections of tumors will be generated to evaluate for differences in mitotic figures and Ki67 index.

Similar experiments can be performed with any other drug disclosed herein, including A1331852 and others.

### REFERENCES

Abou-Alfa G K, Mayer R, Venook A P, et al. Phase II Multicenter, Open-Label Study of Oral ENMD-2076 for the Treatment of Patients with Advanced Fibrolamellar Carcinoma. Oncologist 2020; 25: e1837-e1845.
Bauer J, Kohler N, Maringer Y, et al. The oncogenic fusion protein DNAJB1-PRKACA can be specifically targeted by peptide-based immunotherapy in fibrolamellar hepatocellular carcinoma. Nat Commun 2022; 13:6401.
Benjamini Y, Drai D, Elmer G, et al. Controlling the false discovery rate in behavior genetics research. Behav Brain Res 2001; 125:279-84.
Chipumuro E, Marco E, Christensen C L, et al. CDK7 inhibition suppresses super-enhancer-linked oncogenic transcription in MYCN-driven cancer. Cell 2014; 159:1126-1139.
Christensen C L, Kwiatkowski N, Abraham B J, et al. Targeting transcriptional addictions in small cell lung cancer with a covalent CDK7 inhibitor. Cancer Cell 2014; 26:909-922.
Concordet J P, Haeussler M. CRISPOR: intuitive guide selection for CRISPR/Cas9 genome editing experiments and screens. Nucleic Acids Res 2018; 46:W242-W245.
Courel M, El Yamani F Z, Alexandre D, et al. Secretogranin II is overexpressed in advanced prostate cancer and promotes the neuroendocrine differentiation of prostate cancer cells. Eur J Cancer 2014; 50:3039-49.
Dinh T A, Jewell M L, Kanke M, et al. MicroRNA-375 Suppresses the Growth and Invasion of Fibrolamellar Carcinoma. Cell Mol Gastroenterol Hepatol 2019; 7:803-817.
Dinh T A, Sritharan R, Smith F D, et al. Hotspots of Aberrant Enhancer Activity in Fibrolamellar Carcinoma Reveal Candidate Oncogenic Pathways and Therapeutic Vulnerabilities. Cell Rep 2020; 31:107509.
Dinh T A, Utria A F, Barry K C, et al. A framework for fibrolamellar carcinoma research and clinical trials. Nat Rev Gastroenterol Hepatol 2022; 19:328-342.
Ebmeier C C, Erickson B, Allen B L, et al. Human TFIIH Kinase CDK7 Regulates Transcription-Associated Chromatin Modifications. Cell Rep 2017; 20:1173-1186.
Eggert T, McGlynn K A, Duffy A, et al. Fibrolamellar hepatocellular carcinoma in the USA, 2000-2010: A detailed report on frequency, treatment and outcome based on the Surveillance, Epidemiology, and End Results database. United European Gastroenterol J 2013; 1:351-7.
Eick D, Geyer M. The RNA polymerase II carboxy-terminal domain (CTD) code. Chem Rev. 2013 Nov. 13; 113 (11):8456-90.
El Dika I, Mayer R J, Venook A P, et al. A Multicenter Randomized Three-Arm Phase II Study of (1) Everolimus, (2) Estrogen Deprivation Therapy (EDT) with Leuprolide+Letrozole, and (3) Everolimus+EDT in Patients with Unresectable Fibrolamellar Carcinoma. Oncologist 2020; 25:925-e1603.
Fagerberg L, Hallstrom B M, Oksvold P, et al. Analysis of the human tissue-specific expression by genome-wide integration of transcriptomics and antibody-based proteomics. Mol Cell Proteomics 2014; 13:397-406.
Fisher R P. Cdk7: a kinase at the core of transcription and in the crosshairs of cancer drug discovery. Transcription 2019; 10:47-56.
Florou V, Puri S, Garrido-Laguna I, et al. Considerations for immunotherapy in patients with cancer and comorbid immune dysfunction. Ann Transl Med 2021; 9:1035.
Francisco A B, Kanke M, Massa A P, Dinh T A, Sritharan R, Vakili K, Bardeesy N, Sethupathy P. Multiomic analysis of microRNA-mediated regulation reveals a proliferative axis involving miR-10b in fibrolamellar carcinoma. JCI Insight. 2022 Jun. 8; 7 (11):e154743.
Gaillard E, Bruck N, Brelivet Y, et al. Phosphorylation by PKA potentiates retinoic acid receptor alpha activity by means of increasing interaction with and phosphorylation by cyclin H/cdk7. Proc Natl Acad Sci USA 2006; 103:9548-53.
Gritti I, Wan J, Weeresekara V, Vaz J M, Tarantino G, Bryde T H, Vijay V, Kammula A V, Kattel P, Zhu S, Vu P, Chan M, Wu M J, Gordan J D, Patra K C, Silveira V S, Manguso R T, Wein M N, Ott C J, Qi J, Liu D, Sakamoto K, Gujral T S, Bardeesy N. DNAJB1-PRKACA Fusion Drives Fibrolamellar Liver Cancer through Impaired SIK Signaling and CRTC2/p300-Mediated Transcriptional Reprogramming. Cancer Discov. 2025 Feb. 7; 15 (2):382-400.
Henry S. H. J L, Sawant P, Lefkovith A, Ke N, Dworakowski W, Hodgson G. 13P SY-5609, a highly potent and selective oral CDK7 inhibitor, exhibits robust antitumor activity in preclinical models of KRAS mutant cancers as a single agent and in combination with chemotherapy. Annals of Oncology 2021; 32:S364.
Honeyman J N, Simon E P, Robine N, et al. Detection of a recurrent DNAJB1-PRKACA chimeric transcript in fibrolamellar hepatocellular carcinoma. Science 2014; 343:1010-4.
Hnisz D, Abraham B J, Lee T I, et al. Super-enhancers in the control of cell identity and disease. Cell 2013; 155:934-47.
Hu S, Marineau J J, Rajagopal N, et al. Discovery and Characterization of SY-1365, a Selective, Covalent Inhibitor of CDK7. Cancer Res 2019; 79:3479-3491.
Ianevski A, Giri A K, Aittokallio T. SynergyFinder 3.0: an interactive analysis and consensus interpretation of multi-drug synergies across multiple samples. Nucleic Acids Res 2022; 50:W739-W743.
Ianevski A, He L, Aittokallio T, et al. SynergyFinder: a web application for analyzing drug combination dose-response matrix data. Bioinformatics 2017; 33:2413-2415.
Jiang H, Lei R, Ding S W, et al. Skewer: a fast and accurate adapter trimmer for next-generation sequencing paired-end reads. BMC Bioinformatics 2014; 15:182.
Kastenhuber E R, Lalazar G, Houlihan S L, et al. DNAJB1-PRKACA fusion kinase interacts with beta-catenin and the liver regenerative response to drive fibrolamellar hepatocellular carcinoma. Proc Natl Acad Sci USA 2017; 114:13076-13084.
Kegel V, Deharde D, Pfeiffer E, et al. Protocol for Isolation of Primary Human Hepatocytes and Corresponding Major Populations of Non-parenchymal Liver Cells. J Vis Exp 2016: e53069.
Kwiatkowski N, Zhang T, Rahl P B, et al. Targeting transcription regulation in cancer with a covalent CDK7 inhibitor. Nature 2014; 511:616-20.
Lalazar G, Requena D, Ramos-Espiritu L, Ng D, Bhola P D, de Jong Y P, Wang R, Narayan N J C, Shebl B, Levin S, Michailidis E, Kabbani M, Vercauteren K O A, Hurley A M, Farber B A, Hammond W J, Saltsman J A 3rd, Weinberg E M, Glickman J F, Lyons B A, Ellison J, Schadde E, Hertl M, Leiting J L, Truty M J, Smoot R L, Tierney F, Kato T, Wendel H G, LaQuaglia M P, Rice C M, Letai A, Coffino P, Torbenson M S, Ortiz M V, Simon S M. Identification of Novel Therapeutic Targets for Fibrolamellar Carcinoma Using Patient-Derived Xenografts and Direct-from-Patient Screening. Cancer Discov. 2021 October; 11 (10):2544-2563.
Larochelle S, Amat R, Glover-Cutter K, et al. Cyclin-dependent kinase control of the initiation-to-elongation switch of RNA polymerase II. Nat Struct Mol Biol 2012; 19:1108-15.
Larochelle S, Batliner J, Gamble M J, et al. Dichotomous but stringent substrate selection by the dual-function Cdk7 complex revealed by chemical genetics. Nat Struct Mol Biol 2006; 13:55-62.
Lin M J, Svensson-Arvelund J, Lubitz G S, et al. Cancer vaccines: the next immunotherapy frontier. Nat Cancer 2022; 3:911-926.
Lolli G, Johnson L N. CAK-Cyclin-dependent Activating Kinase: a key kinase in cell cycle control and a target for drugs? Cell Cycle 2005; 4:572-7.
Lopez-Terrada D, Cheung S W, Finegold M J, et al. Hep G2 is a hepatoblastoma-derived cell line. Hum Pathol 2009; 40:1512-5.
Ma R K, Tsai P Y, Farghli A R, Shumway A, Kanke M, Gordan J D, Gujral T S, Vakili K, Nukaya M, Noetzli L, Ronnekleiv-Kelly S, Broom W, Barrow J, Sethupathy P. DNAJB1-PRKACA fusion protein-regulated LINC00473 promotes tumor growth and alters mitochondrial fitness in fibrolamellar carcinoma. PLoS Genet. 2024 Mar. 21; 20 (3):e1011216.
Marineau J J, Hamman K B, Hu S, et al. Discovery of SY-5609: A Selective, Noncovalent Inhibitor of CDK7. J Med Chem 2022; 65:1458-1480.
Neumayer C, Ng D, Jiang C S, et al. Oncogenic Addiction of Fibrolamellar Hepatocellular Carcinoma to the Fusion Kinase DNAJB1-PRKACA. Clin Cancer Res 2023; 29:271-278.
Neumayr C, Haberle V, Serebreni L, Karner K, Hendy O, Boija A, Henninger J E, Li C H, Stejskal K, Lin G, Bergauer K, Pagani M, Rath M, Mechtler K, Arnold C D, Stark A. Differential cofactor dependencies define distinct types of human enhancers. Nature. 2022 June; 606 (7913):406-413.
Nishida-Aoki N, Bondesson A J, Gujral T S. Measuring Real-time Drug Response in Organotypic Tumor Tissue Slices. J Vis Exp. 2020 May 2; (159).
Nukaya M C, C; Zahed, K; Yun, I; Al-Adra, D P; Kazim, N A; Farghli, A R; Chan, M; Kratz, J D; Berres, M E; Yen, A; Gujral, T S; Sethupathy, P; Bradfield, C A; Ronnekleiv-Kelly, S M. CDK7 is a Novel Therapeutic Vulnerability in Fibrolamellar Carcinoma. BioRxiv 2023.
O'Neill A F, Church A J, Perez-Atayde A R, et al. Fibrolamellar carcinoma: An entity all its own. Curr Probl Cancer 2021; 45:100770.
Oikawa T, Wauthier E, Dinh T A, et al. Model of fibrolamellar hepatocellular carcinomas reveals striking enrichment in cancer stem cells. Nat Commun 2015; 6:8070.
Oliver F J, de la Rubia G, Rolli V, et al. Importance of poly(ADP-ribose) polymerase and its cleavage in apoptosis. Lesson from an uncleavable mutant. J Biol Chem 1998; 273:33533-9.
Olson C M, Liang Y, Leggett A, et al. Development of a Selective CDK7 Covalent Inhibitor Reveals Predominant Cell-Cycle Phenotype. Cell Chem Biol 2019; 26:792-803 e10.
Patt Y Z, Hassan M M, Lozano R D, et al. Phase II trial of systemic continuous fluorouracil and subcutaneous recombinant interferon Alfa-2b for treatment of hepatocellular carcinoma. J Clin Oncol 2003; 21:421-7.
Peissert S, Schlosser A, Kendel R, et al. Structural basis for CDK7 activation by MAT1 and Cyclin H. Proc Natl Acad Sci USA 2020; 117:26739-26748.
Robinson M D, McCarthy D J, Smyth G K. edgeR: a Bioconductor package for differential expression analysis of digital gene expression data. Bioinformatics 2010; 26:139-40.
Robinson M D, Oshlack A. A scaling normalization method for differential expression analysis of RNA-seq data. Genome Biol 2010; 11:R25.
Reid P D, Cifu A S, Bass A R. Management of Immunotherapy-Related Toxicities in Patients Treated With Immune Checkpoint Inhibitor Therapy. JAMA 2021; 325:482-483.
Schwartz P B, Nukaya M, Berres M E, et al. The circadian clock is disrupted in pancreatic cancer. PLoS Genet 2023; 19:e1010770.
Schwartz P B, Walcheck M T, Berres M, et al. Chronic jetlag-induced alterations in pancreatic diurnal gene expression. Physiol Genomics 2021; 53:319-335.
Shebl B, Ng D, Lalazar G, et al. Targeting BCL-XL in fibrolamellar hepatocellular carcinoma. JCI Insight 2022; 7.
Sher S, Whipp E, Walker J, et al. VIP152 is a selective CDK9 inhibitor with pre-clinical in vitro and in vivo efficacy in chronic lymphocytic leukemia. Leukemia 2023; 37:326-338.
Shirani M, Levin S, Shebl B, Requena D, Finkelstein T M, Johnson D S, Ng D, Lalazar G, Heissel S, Hojrup P, Molina H, de Jong Y P, Rice C M, Singhi A D, Torbenson M S, Coffino P, Lyons B, Simon S M. Increased Protein Kinase A Activity Induces Fibrolamellar Hepatocellular Carcinoma Features Independent of DNAJB1. Cancer Res. 2024 Aug. 15; 84 (16):2626-2644.
Simon S M. Fighting rare cancers: lessons from fibrolamellar hepatocellular carcinoma. Nat Rev Cancer 2023:1-12.
Sivakumar R, Chan M, Shin J S, Nishida-Aoki N, Kenerson H L, Elemento O, Beltran H, Yeung R, Gujral T S. Organotypic tumor slice cultures provide a versatile platform for immuno-oncology and drug discovery. Oncoimmunology. 2019 Oct. 10; 8 (12):e1670019.
Tewari M, Quan L T, O'Rourke K, et al. Yama/CPP32 beta, a mammalian homolog of CED-3, is a CrmA-inhibitable protease that cleaves the death substrate poly(ADP-ribose) polymerase. Cell 1995; 81:801-9.
Turnham R E, Smith F D, Kenerson H L, Omar M H, Golkowski M, Garcia I, Bauer R, Lau H T, Sullivan K M, Langeberg L K, Ong S E, Riehle K J, Yeung R S, Scott J D. An acquired scaffolding function of the DNAJ-PKAc fusion contributes to oncogenic signaling in fibrolamellar carcinoma. Elife. 2019 May 7; 8:e44187.
Vervoort S J, Welsh S A, Devlin J R, Barbieri E, Knight D A, Offley S, Bjelosevic S, Costacurta M, Todorovski I, Kearney C J, Sandow J J, Fan Z, Blyth B, McLeod V, Vissers J H A, Pavic K, Martin B P, Gregory G, Demosthenous E, Zethoven M, Kong I Y, Hawkins E D, Hogg S J, Kelly M J, Newbold A, Simpson K J, Kauko O, Harvey K F, Ohlmeyer M, Westermarck J, Gray N, Gardini A, Johnstone R W. The PP2A-Integrator-CDK9 axis fine-tunes transcription and can be targeted therapeutically in cancer. Cell. 2021 Jun. 10; 184 (12):3143-3162.e32.
Wang Y, Lu J J, He L, Yu Q. Triptolide (TPL) inhibits global transcription by inducing proteasome-dependent degradation of RNA polymerase II (Pol II). PLoS One. 2011; 6 (9):e23993.
Wu T, Hu E, Xu S, et al. clusterProfiler 4.0: A universal enrichment tool for interpreting omics data. Innovation (Camb) 2021; 2:100141.
Yankulov K Y, Bentley D L. Regulation of CDK7 substrate specificity by MAT1 and TFIIH. EMBO J 1997; 16:1638-46.
Zhang J Z, Lu T W, Stolerman L M, et al. Phase Separation of a PKA Regulatory Subunit Controls cAMP Compartmentation and Oncogenic Signaling. Cell 2020; 182:1531-1544 e15.

What is claimed is:

1. A method of treating a DNAJB1-PRKACA gene fusion-driven cancer in a subject, the method comprising administering a CDK7 inhibitor to the subject in an amount effective to treat the cancer.

2. The method of claim 1, wherein the cancer comprises a liver cancer, a pancreatic cancer, a cholangiocarcinoma (bile duct cancer), or a combination thereof.

3. The method of claim 1, wherein the cancer comprises fibrolamellar hepatocellular carcinoma.

4. The method of claim 1, wherein the CDK7 inhibitor comprises SY-5609, YKL-5-124, samuraciclib, or an enantiomer, a pharmaceutically acceptable salt, and/or a solvate of SY-5609, YKL-5-124, or samuraciclib.

5. The method of claim 1, further comprising administering to the subject one or more additional active agents comprising one or more of a CDK9 inhibitor and a B-cell lymphoma-extra large (Bcl-xL) inhibitor.

6. The method of claim 5, wherein the one or more additional active agents comprise the CDK9 inhibitor.

7. The method of claim 6, wherein the CDK9 inhibitor comprises VIP-152, NVP-2, or an enantiomer, a pharmaceutically acceptable salt, and/or a solvate of VIP-152 or NVP-2.

8. The method of claim 7, wherein the CDK7 inhibitor comprises SY-5609, YKL-5-124, samuraciclib, or an enantiomer, a pharmaceutically acceptable salt, and/or a solvate of SY-5609, YKL-5-124, or samuraciclib.

9. The method of claim 5, wherein the one or more additional active agents comprise the BCL-xL inhibitor.

10. The method of claim 9, wherein the BCL-XL inhibitor comprises A1331852 or an enantiomer, a pharmaceutically acceptable salt, and/or a solvate thereof.

11. The method of claim 10, wherein the CDK7 inhibitor comprises SY-5609, YKL-5-124, samuraciclib, or an enantiomer, a pharmaceutically acceptable salt, and/or a solvate of SY-5609, YKL-5-124, or samuraciclib.

12. The method of claim 5, wherein the CDK7 inhibitor is administered to the subject within a week of administering at least one of the one or more additional active agents.

13. The method of claim 5, wherein the CDK7 inhibitor and at least one of the one or more additional active agents are simultaneously administered to the subject.

14. A composition comprising a CDK7 inhibitor and one or more additional active agents comprising one or more of a CDK9 inhibitor and a B-cell lymphoma-extra large (Bcl-xL) inhibitor.

15. The composition of claim 14, wherein the CDK7 inhibitor comprises SY-5609, YKL-5-124, samuraciclib, or an enantiomer, a pharmaceutically acceptable salt, and/or a solvate of SY-5609, YKL-5-124, or samuraciclib.

16. The composition of claim 14, wherein the one or more additional active agents comprise the CDK9 inhibitor.

17. The composition of claim 16, wherein the CDK9 inhibitor comprises VIP-152, NVP-2, or an enantiomer, a pharmaceutically acceptable salt, and/or a solvate of VIP-152 or NVP-2.

18. The composition of claim 14, wherein the one or more additional active agents comprise the BCL-xL inhibitor.

19. The composition of claim 18, wherein the BCL-xL inhibitor comprises A1331852 or an enantiomer, a pharmaceutically acceptable salt, and/or a solvate thereof.

20. The composition of claim 14, wherein:
the CDK7 inhibitor comprises SY-5609, YKL-5-124, samuraciclib, or an enantiomer, a pharmaceutically acceptable salt, and/or a solvate of SY-5609, YKL-5-124, or samuraciclib; and
the one or more additional active agents comprise one or more of:
VIP-152, NVP-2, or an enantiomer, a pharmaceutically acceptable salt, and/or a solvate of VIP-152 or NVP-2: and
A1331852 or an enantiomer, a pharmaceutically acceptable salt, and/or a solvate thereof.


SEQUENCE LISTING (11 sequences)

SEQ ID NO: 1 - DNA, 20 bp, synthetic construct
  source 1..20: mol_type = other DNA
caggagccgaccccgttcgt

SEQ ID NO: 2 - DNA, 20 bp, synthetic construct
  source 1..20: mol_type = other DNA
gtagacgcggttgcgctaag

SEQ ID NO: 3 - DNA, 20 bp, synthetic construct
  source 1..20: mol_type = other DNA
agcttctagcatgtctgggg

SEQ ID NO: 4 - DNA, 21 bp, synthetic construct
  source 1..21: mol_type = other DNA
ctgggaaggctcatgagacct

SEQ ID NO: 5 - DNA, 20 bp, synthetic construct
  source 1..20: mol_type = other DNA
gttcaaggagatcgctgagg

SEQ ID NO: 6 - DNA, 20 bp, synthetic construct
  source 1..20: mol_type = other DNA
ttcccggtctccttgtgttt

SEQ ID NO: 7 - DNA, 18 bp, synthetic construct
  source 1..18: mol_type = other DNA
agcgggactttcccattt

SEQ ID NO: 8 - DNA, 17 bp, synthetic construct
  source 1..17: mol_type = other DNA
aagaagggcagcgagca

SEQ ID NO: 9 - DNA, 27 bp, synthetic construct
  source 1..27: mol_type = other DNA
tggctttggctaagaattctttcacgc

SEQ ID NO: 10 - protein, 7 aa, Homo sapiens
YSPTSPS

SEQ ID NO: 11 - protein, 7 aa, synthetic construct
TXXVVTL